(19) United States
(12) Patent Application Publication
(10) Pub. No.: US 2014/0099676 A1
(43) Pub. Date: Apr. 10, 2014

(54) MICROORGANISMS AND METHODS FOR PRODUCING ACRYLATE AND OTHER PRODUCTS FROM HOMOSERINE

(71) Applicants: Jun XU, Mason, OH (US); Charles Winston Saunders, Fairfield, OH (US); Phillip Richard Green, Wyoming, OH (US); Juan Esteban Velasquez, Cincinnati, OH (US)

(72) Inventors: Jun XU, Mason, OH (US); Charles Winston Saunders, Fairfield, OH (US); Phillip Richard Green, Wyoming, OH (US); Juan Esteban Velasquez, Cincinnati, OH (US)

(21) Appl. No.: 13/646,053

(22) Filed: Oct. 5, 2012

(51) Int. Cl. C12P 13/02; C12P 7/42

(52) U.S. Cl. CPC C12P 13/02; C12P 7/42
USPC 435/92; 435/146; 435/158; 435/136; 435/252.33; 435/157

(57) ABSTRACT

This invention relates to microorganisms that convert a carbon source to acrylate or other desirable products using homoserine and 2-keto-4-hydroxybutyrate as intermediates. The invention provides genetically engineered microorganisms that carry out the conversion, as well as methods for producing acrylate by culturing the microorganisms. Also provided are microorganisms and methods for converting homoserine to 3-hydroxypropionyl-CoA, 3-hydroxypropionate (3HP), poly-3-hydroxypropionate and 1,3-propanediol.

### CROSS REFERENCE TO RELATED APPLICATION

This application claims the benefit of U.S. Provisional Application No. 61/543,511 filed Oct. 5, 2011.

### FIELD OF THE INVENTION

This invention relates to microorganisms that convert a carbon source to acrylate or other desirable products using homoserine and 2-keto-4-hydroxybutyrate as intermediates. The invention provides genetically engineered microorganisms that carry out the conversion, as well as methods for producing acrylate by culturing the microorganisms. Also provided are microorganisms and methods for converting homoserine to 3-hydroxypropionyl-CoA, 3-hydroxypropionate (3HP), poly-3-hydroxypropionate and 1,3-propanediol.

### BACKGROUND OF THE INVENTION

One organic chemical used to make super absorbent polymers (used in diapers), plastics, coatings, paints, adhesives, and binders (used in leather, paper and textile products) is acrylic acid. Acrylic acid (IUPAC: prop-2-enoic acid) is the simplest unsaturated carboxylic acid.

Traditionally, acrylic acid is made from propene. Propene itself is a byproduct of oil refining from petroleum (i.e., crude oil) and of natural gas production. Disadvantages associated with traditional acrylic acid production are that petroleum is a nonrenewable starting material and that the oil refining process pollutes the environment. Synthesis methods for acrylic acid utilizing other starting materials have not been adopted for widespread use due to expense or environmental concerns. These starting materials included, for example, acetylene, ethenone and ethylene cyanohydrins.

To avoid petroleum-based production, researchers have proposed other methods for producing acrylic acid involving the fermentation of sugars by engineered microorganisms. Straathof et al., Appl Microbiol Biotechnol, 67: 727-734 (2005) discusses a conceptual fermentation process for acrylic acid production from sugars. The process proposed in the article proceeds via a β-alanine, methylcitrate, malonyl-CoA or methylmalonate-CoA intermediate in the microorganism. Another process described in Lynch, U.S. Patent Publication No. 2011/0125118 relates to using synthesis gas components as a carbon source in a microbial system to produce 3-hydroxypropionic acid, with subsequent conversion of the 3-hydroxyproprionic acid to acrylic acid.

Methods to manufacture other organic chemicals in genetically engineered microorganisms have been proposed. See, for example, U.S. Patent Publication No. 2011/0014669 published Jan. 20, 2011 relating to microorganisms for converting L-glutamate to 1,4-butanediol.

Since at least four million metric tons of acrylic acid are produced annually, there remains a need in the art for cost-effective, environmentally-friendly methods for its production from renewable carbon sources.

### SUMMARY OF THE INVENTION

Homoserine is an intermediate in the biosynthesis of the amino acids threonine and methionine. Homoserine is naturally made from glucose in the bacterium E. coli and many other organisms. FIG. 1 set out an illustration of the steps converting glucose to homoserine.

The present invention utilizes homoserine and 2-keto-4-hydroxybutyrate as intermediates to make acrylate (the chemical form of acrylic acid at neutral pH) and other products of interest. FIGS. 2 and 3 set out examples of contemplated pathways for making acrylate, 3-hydroxypropionate, poly-3-hydroxypropionate, 1,3-propanediol and 3-hydroxypropionyl-CoA from homoserine. Microorganisms do not naturally make acrylate and the other products, but microorganisms (such as bacteria, yeast, fungi and algae) are genetically modified according to the invention to carry out the conversions in the pathways. Microorganisms include, but are not limited to, an E. coli bacterium.

### Producing Acrylate

In a first aspect, the invention provides a first type of microorganism, one that converts homoserine to acrylate, wherein the microorganism expresses recombinant genes encoding a deaminase or transaminase; a dehydrogenase or decarboxylase; a dehydratase; and a thioesterase, a phosphate transferase/kinase combination, or an acyl-CoA transferase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, and an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are respectively set out in SEQ ID NOs: 17 and 19.

The dehydrogenase catalyzes a reaction to convert 2-keto-4-hydroxybutyrate to 3-hydroxypropionyl-CoA. In some embodiments, the dehydrogenase is a 2-keto acid dehydrogenase (or an alpha keto acid dehydrogenase). Dehydrogenases include, but are not limited to, a pyruvate dehydrogenase, a 2-keto-glutarate dehydrogenase or a branched chain keto acid dehydrogenase. A pyruvate dehydrogenase known in the art is the pyruvate dehydrogenase PDH, the amino acid sequences of the subunits of which are set out in SEQ ID NOs: 30, 32 and 34. Exemplary DNA sequences encoding those subunits are respectively set out in SEQ ID NOs: 29, 31 and 33. A 2-keto-glutarate dehydrogenase known in the art similarly comprises three subunits, the amino acid sequences of which are set out in SEQ ID NOs: 36, 38 and 40. Exemplary DNA sequences encoding those subunits are respectively set out in SEQ ID NOs: 35, 37 and 39. A branched chain keto acid dehydrogenase known in the art is the branched chain keto acid dehydrogenase BKD, the amino acid sequences of the subunits of which are set out in SEQ ID NOs: 22, 24, 26 and 28. Exemplary DNA sequences encoding those subunits are respectively set out in SEQ ID NOs: 21, 23, 25 and 27.

The dehydratase catalyzes a reaction to convert 3-hydroxypropionyl-CoA to acryloyl-CoA. In some embodiments, the dehydratase is a 3-hydroxypropionyl-CoA-dehydratase. The amino acid sequence of a 3-hydroxypropionyl-CoA-dehydratase known in the art is set out in SEQ ID NO: 48. An exemplary DNA sequence encoding the 3-hydroxypropionyl-CoA-dehydratase is set out in SEQ ID NO: 47.

The thioesterase, the phosphate transferase/kinase combination or the acyl-CoA transferase catalyzes a reaction to convert acryloyl-CoA to acrylate. In some embodiments, the thioesterase is acryloyl-CoA thioesterase. The amino acid sequence of a phosphate acryloyltransferase known in the art is set out in SEQ ID NO: 50. An exemplary DNA sequence encoding the phosphate acryloyltransferase is SEQ ID NO: 49. The amino acid sequence of an acrylate kinase known in the art is set out in SEQ ID NO: 52. An exemplary DNA sequence encoding the acrylate kinase is set out in SEQ ID NO: 51. The amino acid sequence of an acyl-CoA transferase known in the art is set out in SEQ ID NO: 46. An exemplary DNA sequence encoding the acyl-CoA transferase is set out in SEQ ID NO: 45.

In a second aspect, the invention provides a first type of method, one for producing acrylate in which the first type of microorganism is cultured to produce acrylate. The first type of method for producing acrylate converts homoserine to 2-keto-4-hydroxybutyrate, 2-keto-4-hydroxybutyrate to 3-hydroxypropionyl-CoA, 3-hydroxypropionyl-CoA to acryloyl-CoA and then acryloyl-CoA to acrylate.

In a third aspect, the invention provides a second type of microorganism, one that converts homoserine to acrylate, wherein the microorganism expresses recombinant genes encoding: a deaminase or transaminase, a decarboxylase, a dehydrogenase, a dehydratase and a thioesterase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The decarboxylase catalyzes a reaction to convert 2-keto-4-hydroxybutyrate to 3-hydroxy-propionaldehyde. In some embodiments, the decarboxylase is a 2-keto acid decarboxylase. The 2-keto acid decarboxylases include, but are not limited to, the 2-keto acid decarboxylase KdcA set out in SEQ ID NO: 54 and its derivatives. An exemplary DNA sequence encoding KdcA is set out in SEQ ID NO: 53.

The dehydrogenase catalyzes a reaction to convert 3-hydroxy-propionaldehyde to 3-hydroxypropionyl-CoA. In some embodiments, the dehydrogenase is a propionaldehyde dehydrogenase. Propionaldehyde dehydrogenases include, but are not limited to, a PduP. Amino acid sequences of some PduP propionaldehyde dehydrogenases known in the art are set out in SEQ ID NOs: 60 and 62. Exemplary DNA sequences encoding the PduP propionaldehyde dehydrogenases are respectively set out in SEQ ID NOs: 59 and 61.

The dehydratase catalyzes a reaction to convert 3-hydroxypropionyl-CoA to acryloyl-CoA. In some embodiments, the dehydratase is a 3-hydroxypropionyl-CoA dehydratase. The amino acid sequence of 3-hydroxypropionyl-CoA dehydratase known in the art is set out in SEQ ID NO: 48. An exemplary DNA sequence encoding the 3-hydroxypropionyl-CoA dehydratase is set out in SEQ ID NO: 47.

The thioesterase catalyzes a reaction to convert acryloyl-CoA to acrylate. In some embodiments, the thioesterase is an acryloyl-CoA thioesterase. Acryloyl-CoA thioesterases include, but are not limited to E. coli TesB set out in SEQ ID NO: 90, the Clostridium propionicum-derived thioesterase including an E324D substitution set out in SEQ ID NO: 92 and the Megasphaera elsdenii-derived thioesterase including an E325D substitution set out in SEQ ID NO: 94. Exemplary DNA sequences encoding these acryloyl-CoA thioesterases are respectively set out in SEQ ID NOs: 89, 91 (codon-optimized for E. coli) and 93 (codon-optimized for E. coli).

In a fourth aspect, the invention provides a second type of method, one for producing acrylate in which the second type of microorganism is cultured to produce acrylate. The second type of method for producing acrylate converts homoserine to 2-keto-4-hydroxybutyrate, 2-keto-4-hydroxybutyrate to 3-hydroxy-propionaldehyde, 3-hydroxy-propionaldehyde to 3-hydroxypropionyl-CoA, 3-hydroxy-propionyl-CoA to acryloyl-CoA and then acryloyl-CoA to acrylate.

Producing 3-hydroxypropionate

In a fifth aspect, the invention provides a third type of microorganism, one that converts homoserine to 3-hydroxypropionate, wherein the microorganism expresses recombinant genes encoding: a deaminase or transaminase, a dehydrogenase or decarboxylase, and acyl-CoA transferase or athioesterase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The dehydrogenase or decarboxylase catalyzes a reaction to convert 2-keto-4-hydroxybutyrate to 3-hydroxypropionyl-CoA. In some embodiments, the dehydrogenase is a 2-keto acid dehydrogenase (or an alpha keto acid dehydrogenase). Dehydrogenases include, but are not limited to, a pyruvate dehydrogenase, a 2-keto-glutarate dehydrogenase or a branched chain keto acid dehydrogenase. A pyruvate dehydrogenase known in the art is the pyruvate dehydrogenase PDH, the amino acid sequences of the subunits of which are set out in SEQ ID NOs: 30, 32 and 34. Exemplary DNA sequences encoding those subunits are respectively set out in SEQ ID NOs: 29, 31 and 33. A 2-keto-glutarate dehydrogenase known in the art similarly comprises three subunits, the amino acid sequences of which are set out in SEQ ID NOs: 36, 38 and 40. Exemplary DNA sequences encoding those subunits are respectively set out in SEQ ID NOs: 35, 37 and 39. A branched chain keto acid dehydrogenase known in the art is the branched chain keto acid dehydrogenase BKD, the amino acid sequences of the subunits of which are set out in SEQ ID NOs: 22, 24, 26 and 28. Exemplary DNA sequences encoding those subunits are respectively set out in SEQ ID NOs: 21, 23, 25 and 27.

The acyl-CoA transferase or the acyl-CoA thioesterase catalyzes a reaction to convert 3-hydroxypropionyl-CoA to 3-hydroxypropionate. Contemplated thioesterases include, but are not limited to E. coli TesB set out in SEQ ID NO: 90, the C. propionicum-derived thioesterase including an E324D substitution set out in SEQ ID NO: 92 and the M. elsdenii-derived thioesterase including an E325D substitution set out in SEQ ID NO: 94. Exemplary (codon-optimized for E. coli) DNA sequences encoding these thioesterases are respectively set out in SEQ ID NOs: 89, 91 (codon-optimized for E. coli) and 93 (codon-optimized for E. coli).

In a sixth aspect, the invention provides a third type of method, one for producing 3-hydroxypropionate in which the third type of microorganism is cultured to produce 3-hydroxypropionate. The third type of method converts homoserine to 2-keto-4-hydroxybutyrate, 2-keto-4-hydroxybutyrate to 3-hydroxy-propionaldehyde, 3-hydroxy-propionaldehyde to 3-hydroxypropionyl-CoA and then 3-hydroxypropionyl-CoA to 3-hydroxypropionate.

In a seventh aspect, the invention provides a fourth type of microorganism, one that converts homoserine to 3-hydroxypropionate, wherein the microorganism expresses recombinant genes encoding: a deaminase or transaminase, a decarboxylase and a dehydrogenase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The decarboxylase catalyzes a reaction to convert 2-keto-4-hydroxybutyrate to 3-hydroxy-propionaldehyde. In some embodiments the decarboxylase is a 2-keto acid decarboxylase. The 2-keto acid decarboxylases include, but are not limited to, the 2-keto acid decarboxylase KdcA set out in SEQ ID NO: 54 and its derivatives. An exemplary DNA sequence encoding KdcA is set out in SEQ ID NO: 53.

The dehydrogenase catalyzes a reaction to convert 3-hydroxy-propionaldehyde to 3-hydroxypropionate. In some embodiments, the dehydrogenase is an aldehyde dehydrogenase. Amino acid sequences of aldehyde dehydrogenases known in the art are set out in SEQ ID NOs: 56 and 58. Exemplary DNA sequences encoding the aldehyde dehydrogenases are respectively set out in SEQ ID NOs: 55 and 57.

In an eighth aspect, the invention provides a fourth type of method, one for producing 3-hydroxypropionate in which the fourth type of microorganism is cultured to produce 3-hydroxypropionate. The fourth type of method converts homoserine to 2-keto-4-hydroxybutyrate, 2-keto-4-hydroxybutyrate to 3-hydroxy-propionaldehyde, 3-hydroxy-propionaldehyde to 3-hydroxypropionate.

In a ninth aspect, the invention provides a fifth type of microorganism, one that converts homoserine to 3-hydroxypropionate, wherein the microorganism expresses recombinant genes encoding: a deaminase or transaminase, a decarboxylase, a dehydrogenase, and a acyl-CoA transferase or a thioesterase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The decarboxylase catalyzes a reaction to convert 2-keto-4-hydroxybutyrate to 3-hydroxy-propionaldehyde. In some embodiments, the decarboxylase is a 2-keto acid decarboxylase. The 2-keto acid decarboxylases include, but are not limited to, the 2-keto acid decarboxylase KdcA set out in SEQ ID NO: 54 and its derivatives. An exemplary DNA sequence encoding KdcA is set out in SEQ ID NO: 53.

The dehydrogenase catalyzes a reaction to convert 3-hydroxy-propionaldehyde to 3-hydroxypropionyl-CoA. In some embodiments, the dehydrogenase is a propionaldehyde dehydrogenase. Propionaldehyde dehydrogenases include, but are not limited to, a PduP. Amino acid sequences of some PduP propionaldehyde dehydrogenases known in the art are set out in SEQ ID NOs: 60 and 62. Exemplary DNA sequences encoding the PduP propionaldehyde dehydrogenases are respectively set out in SEQ ID NOs: 59 and 61.

The 3-hydroxypropionyl-CoA transferase or thioesterase catalyzes a reaction to convert 3-hydroxypropionyl-CoA to 3-hydroxypropionate. Contemplated thioesterases include, but are not limited to E. coli TesB set out in SEQ ID NO: 90, the C. propionicum-derived thioesterase including an E324D substitution set out in SEQ ID NO: 92 and the M. elsdenii-derived thioesterase including an E325D substitution set out in SEQ ID NO: 94. Exemplary DNA sequences encoding these acryloyl-CoA thioesterases are respectively set out in SEQ ID NOs: 89, 91 (codon-optimized for E. coli) and 93 (codon-optimized for E. coli).

In a tenth aspect, the invention provides a fifth type of method, one for producing 3-hydroxypropionate in which the fifth type of microorganism is cultured to produce 3-hydroxypropionate. The fifth type of method for producing acrylate converts homoserine to 2-keto-4-hydroxybutyrate, 2-keto-4-hydroxybutyrate to 3-hydroxy-propionaldehyde, 3-hydroxy-propionaldehyde to 3-hydroxypropionyl-CoA, and 3-hydroxy-propionyl-CoA to 3-hydroxypropionate.

Producing poly-3-hydroxypropionate

In a eleventh aspect, the invention provides a sixth type of microorganism, one that converts homoserine to poly-3-hydroxypropionate, wherein the microorganism expresses recombinant genes encoding: a deaminase or transaminase, a dehydrogenase or decarboxylase, and a PHA synthase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The dehydrogenase or decarboxylase catalyzes a reaction to convert 2-keto-4-hydroxybutyrate to 3-hydroxypropionyl-CoA. In some embodiments, the dehydrogenase is a 2-keto acid dehydrogenase (or an alpha keto acid dehydrogenase). Dehydrogenases include, but are not limited to, a pyruvate dehydrogenase, a 2-keto-glutarate dehydrogenase or a branched chain keto acid dehydrogenase. A pyruvate dehydrogenase known in the art is the pyruvate dehydrogenase PDH, the amino acid sequences of the subunits of which are set out in SEQ ID NOs: 30, 32 and 34. Exemplary DNA sequences encoding those subunits are respectively set out in SEQ ID NOs: 29, 31 and 33. A 2-keto-glutarate dehydrogenase known in the art similarly comprises three subunits, the amino acid sequences of which are set out in SEQ ID NOs: 36, 38 and 40. Exemplary DNA sequences encoding those subunits are respectively set out in SEQ ID NOs: 35, 37 and 39. A branched chain keto acid dehydrogenase known in the art is the branched chain keto acid dehydrogenase BKD, the amino acid sequences of the subunits of which are set out in SEQ ID NOs: 22, 24, 26 and 28. Exemplary DNA sequences encoding those subunits are respectively set out in SEQ ID NOs: 21, 23, 25 and 27.

The PHA synthase catalyzes a reaction to convert 3-hydroxypropionyl-CoA to poly-3-hydroxyalkanoate containing 3-hydroxypropionate monomers. The polymer may have a molecule of Coenzyme A (CoA) at the carboxy end. The amino acid sequence of a PHA synthase known in the art is set out in SEQ ID NO: 42. An exemplary DNA sequence encoding the PHA synthase is set out in SEQ ID NO: 41.

In a twelfth aspect, the invention provides a sixth type of method, one for producing poly-3-hydroxypropionate in which the sixth type of microorganism is cultured to produce poly-3-hydroxypropionate. The sixth type of method converts homoserine to 2-keto-4-hydroxybutyrate, 2-keto-4-hydroxybutyrate to 3-hydroxypropionyl-CoA and 3-hydroxypropionyl-CoA to poly-3-hydroxypropionate.

In thirteenth aspect, the invention provides a seventh type of microorganism, one that converts homoserine to poly-3-hydroxypropionate, wherein the microorganism expresses recombinant genes encoding: a deaminase or transaminase, a decarboxylase, a dehydrogenase and a PHA synthase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The decarboxylase catalyzes a reaction to convert 2-keto-4-hydroxybutyrate to 3-hydroxy-propionaldehyde. In some embodiments, the decarboxylase is a 2-keto acid decarboxylase. The 2-keto acid decarboxylases include, but are not limited to, the 2-keto acid decarboxylase KdcA set out in SEQ ID NO: 54 and its derivatives. An exemplary DNA sequence encoding KdcA is set out in SEQ ID NO: 53.

The dehydrogenase catalyzes a reaction to convert 3-hydroxy-propionaldehyde to 3-hydroxypropionyl-CoA. In some embodiments, the dehydrogenase is a propionaldehyde dehydrogenase. Propionaldehyde dehydrogenases include, but are not limited to, a PduP. Amino acid sequences encoding of PduP propionaldehyde dehydrogenases known in the art are set out in SEQ ID NOs: 60 and 62. Exemplary DNA sequences encoding the PduP propionaldehyde dehydrogenases are respectively set out in SEQ ID NOs: 59 and 61.

The PHA synthase catalyzes a reaction to convert 3-hydroxypropionyl-CoA to poly-3-hydroxyalkanoate containing 3-hydroxypropionate monomers. The polymer may have a molecule of Coenzyme A (CoA) at the carboxy end. The amino acid sequence of a PHA synthase known in the art is set out in SEQ ID NO: 42. An exemplary DNA sequence encoding the PHA synthase is set out in SEQ ID NO: 41.

In a fourteenth aspect, the invention provides a seventh type of method, one for producing poly-3-hydroxypropionate in which the seventh type of microorganism is cultured to produce poly-3-hydroxypropionate. The seventh type of method converts homoserine to 2-keto-4-hydroxybutyrate, 2-keto-4-hydroxybutyrate to 3-hydroxy-propionaldehyde, 3-hydroxy-propionaldehyde to 3-hydroxypropionyl-CoA and then 3-hydroxy-propionyl-CoA to poly-3-hydroxypropionate.

Producing 3-hydroxypropionyl-CoA

In a fifteenth aspect, the invention provides a eighth type of microorganism that converts homoserine to 3-hydroxypropionyl-CoA, wherein the microorganism expresses recombinant genes encoding: a deaminase or transaminase, and a dehydrogenase or decarboxylase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The dehydrogenase or decarboxylase catalyzes a reaction to convert 2-keto-4-hydroxybutyrate to 3-hydroxypropionyl-CoA. In some embodiments, the dehydrogenase is a 2-keto acid dehydrogenase (or an alpha keto acid dehydrogenase). Dehydrogenases include, but are not limited to, a pyruvate dehydrogenase, a 2-keto-glutarate dehydrogenase or a branched chain keto acid dehydrogenase. A pyruvate dehydrogenase known in the art is the pyruvate dehydrogenase PDH, the amino acid sequences of the subunits of which are set out in SEQ ID NOs: 30, 32 and 34. Exemplary DNA sequences encoding those subunits are respectively set out in SEQ ID NOs: 29, 31 and 33. A 2-keto-glutarate dehydrogenase known in the art similarly comprises three subunits, the amino acid sequences of which are set out in SEQ ID NOs: 36, 38 and 40. Exemplary DNA sequences encoding those subunits are respectively set out in SEQ ID NOs: 35, 37 and 39. A branched chain keto acid dehydrogenase known in the art is the branched chain keto acid dehydrogenase BKD, the amino acid sequences of the subunits of which are set out in SEQ ID NOs: 22, 24, 26 and 28. Exemplary DNA sequences encoding those subunits are respectively set out in SEQ ID NOs: 21, 23, 25 and 27.

In a sixteenth aspect, the invention provides an eighth type of method, one for producing 3-hydroxypropionyl-CoA in which the eighth type of microorganism is cultured to produce 3-hydroxypropionyl-CoA. The eighth type of method converts homoserine to 2-keto-4-hydroxybutyrate and then converts 2-keto-4-hydroxybutyrate to 3-hydroxypropionyl-CoA.

In a seventeenth aspect, the invention provides a ninth type of microorganism, one that converts homoserine to 3-hydroxypropionyl-CoA, wherein the microorganism expresses recombinant genes encoding: a deaminase or transaminase, a decarboxylase, and a dehydrogenase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The decarboxylase catalyzes a reaction to convert 2-keto-4-hydroxybutyrate to 3-hydroxy-propionaldehyde. In some embodiments, the decarboxylase is a 2-keto acid decarboxylase. The 2-keto acid decarboxylases include, but are not limited to, the 2-keto acid decarboxylase KdcA set out in SEQ ID NO: 54 and its derivatives. An exemplary DNA sequence encoding KdcA is set out in SEQ ID NO: 53.

The dehydrogenase catalyzes a reaction to convert 3-hydroxy-propionaldehyde to 3-hydroxypropionyl-CoA. In some embodiments, the dehydrogenase is a propionaldehyde dehydrogenase. Propionaldehyde dehydrogenases include, but are not limited to, a PduP. Amino acid sequences encoding of PduP propionaldehyde dehydrogenases known in the art are set out in SEQ ID NOs: 60 and 62. Exemplary DNA sequences encoding the PduP propionaldehyde dehydrogenases are respectively set out in SEQ ID NOs: 59 and 61.

In an eighteenth aspect, the invention provides a ninth type of method, one for producing 3-hydroxypropionyl-CoA in which the ninth type of microorganism is cultured to produce 3-hydroxypropionyl-CoA. The ninth type of method converts homoserine to 2-keto-4-hydroxybutyrate, 2-keto-4-hydroxybutyrate to 3-hydroxy-propionaldehyde, and 3-hydroxy-propionaldehyde to 3-hydroxypropionyl-CoA.

Producing 1,3-propanediol

In a nineteenth aspect, the invention provides an tenth type of microorganism, one that converts homoserine to 1,3-propanediol, wherein the microorganism expresses recombinant genes encoding: a deaminase or transaminase, a decarboxylase and a 1,3-propanediol dehydrogenase or aldehyde reductase.

The deaminase or transaminase catalyzes a reaction to convert homoserine to 2-keto-4-hydroxybutyrate. In some embodiments, the deaminase or transaminase is an aminotransferase, an L-amino acid oxidase or an L-amino acid dehydrogenase. Aminotransferases include, but are not limited to, a glutamate-oxaloacetate aminotransferase, a glutamate-pyruvate aminotransferase, an L-aspartate:2-oxoglutarate aminotransferase, an L-alanine:2-oxoglutarate aminotransferase. Amino acid sequences of some aminotransferases known in the art are set out in SEQ ID NOs: 2, 4, 6, 8, 10 and 12. Exemplary DNA sequences encoding those aminotransferases are respectively set out in SEQ ID NOs: 1, 3, 5, 7, 9 and 11. Amino acid sequences of some L-amino acid oxidases known in the art are set out in SEQ ID NOs: 14 and 16. Exemplary DNA sequences encoding those L-amino acid oxidases are respectively set out in SEQ ID NOs: 13 and 15. Amino acid sequences of some L-amino acid dehydrogenases known in the art are set out in SEQ ID NOs: 18 and 20. Exemplary DNA sequences encoding those L-amino acid dehydrogenases are set out in SEQ ID NOs: 17 and 19.

The decarboxylase catalyzes a reaction to convert 2-keto-4-hydroxybutyrate to 3-hydroxy-propionaldehyde. In some embodiments, the decarboxylase is a 2-keto acid decarboxylase. The 2-keto acid decarboxylases include, but are not limited to, the 2-keto acid decarboxylase KdcA set out in SEQ ID NO: 54 and its derivatives. An exemplary DNA sequence encoding KdcA is set out in SEQ ID NO: 53.

The 1,3-propanediol dehydrogenase or aldehyde reductase catalyzes a reaction to convert 3-hydroxypropionaldehyde to 1,3-propanediol. Amino acid sequences of some 1,3-propanediol dehydrogenases know in the art are set out in SEQ ID NOs: 64, 66, 68, 70 and 72. Exemplary DNA sequence encoding the 1,3-propanediol dehydrogenases are respectively set out in SEQ ID NOs: 63, 65, 67, 69 and 71.

In a twentieth aspect, the invention provides an tenth type of method, one for producing 1,3-propanediol in which the tenth type of microorganism is cultured to produce 1,3-propanediol. The tenth type of method converts homoserine to 2-keto-4-hydroxybutyrate, 2-keto-4-hydroxybutyrate to 3-hydroxy-propionaldehyde and then 3-hydroxy-propionaldehyde to 1,3-propanediol.

### Increasing the Carbon Flow to Homoserine

In a twenty-first aspect, the invention provides microorganisms that include further genetic modifications in order to increase the carbon flow to homoserine which, in turn, increases the production of acrylate or other products of the invention. The microorganisms exhibit one or more of the following characteristics.

In some embodiments, the microorganism exhibits increased carbon flow to oxaloacetate in comparison to a corresponding wild-type microorganism. The microorganism expresses a recombinant gene encoding, for example, phosphoenolpyruvate carboxylase or pyruvate carboxylase (or both). The phosphoenolpyruvate caroxylases include, but are not limited to, the phosphoenolpyruvate carboxylase set out in SEQ ID NO: 84. An exemplary DNA sequence encoding the phosphoenolpyruvate carboxylase is set out in SEQ ID NO: 83. The pyruvate carboxylases include, but are not limited to, the pyruvate carboxylases set out in SEQ ID NOs: 86 and 88. Exemplary DNA sequences encoding the pyruvate carboxylases are set out in SEQ ID NO: 85 and 87.

In some embodiments, the microorganism exhibits reduced aspartate kinase feedback inhibition in comparison to a corresponding wild-type microorganism. The microorganism expresses one or more of the genes encoding the polypeptides including, but not limited to, S345F ThrA (SEQ ID NO: 76), T352I LysC (SEQ ID NO: 78) and MetL (SEQ ID NO: 74). Exemplary coding sequences encoding the polypeptides are respectively set out in SEQ ID NO: 75, SEQ ID NO: 77 and SEQ ID NO: 73.

In some embodiments, the microorganism exhibits reduced lysA gene expression or diaminopimelate decarboxylase activity in comparison to a corresponding wild-type microorganism. In some embodiments, the microorganism exhibits reduced dapA expression or dihydropicolinate synthase activity in comparison to a corresponding wild type organism. An exemplary DNA sequence of a lysA coding sequence known in the art is set out in SEQ ID NO: 113. It encodes the amino acid sequence set out in SEQ ID NO: 114. An exemplary DNA sequence of a dapA coding sequence known in the art is set out in SEQ ID NO: 115. It encodes the amino acid sequence set out in SEQ ID NO: 116.

In some embodiments, the microorganism exhibits reduced metA gene expression or homoserine succinyltransferase activity in comparison to a corresponding wild-type microorganism. An exemplary DNA sequence of a metA coding sequence known in the art is set out in SEQ ID NO: 79. It encodes the amino acid sequence set out in SEQ ID NO: 80.

In some embodiments, the microorganism exhibits reduced thrB gene expression or homoserine kinase activity in comparison to a corresponding wild-type microorganism. An exemplary DNA sequence of a thrB coding sequence known in the art is set out in SEQ ID NO: 81. It encodes the amino acid sequence set out in SEQ ID NO: 82.

In some embodiments, the microorganism does not express an eda gene. An exemplary DNA sequence of an eda coding sequence known in the art is set out in SEQ ID NO: 43. It encodes the amino acid sequence set out in SEQ ID NO: 44.

In an twenty-second aspect, the invention provides an methods of culturing the further modified microorganisms to produce products of the invention.

### Thioesterases

In a twenty-third aspect, the invention provides a thioesterase that hydrolyzes an intermediate of a metabolic pathway described herein to produce a desired end product. In this regard, a microorganism of the invention expresses a recombinant gene comprising a nucleic acid sequence encoding a thioesterase with activity against Coenzyme A (CoA) attached to a two-, three- or four-carbon chain, such as a three- or four-carbon chain comprising a double bond (e.g., a three- or four-carbon chain comprising a double bond between C2 and C3). In some embodiments, the thioesterase hydrolyzes acryloyl-CoA to form acrylic acid. Alternatively (or in addition), in some embodiments the thioesterase hydrolyzes crotonoyl-CoA to form crotonic acid.

This aspect of the invention is predicated, at least in part, on the use of thioesterases with activity against substrates with short carbon chains (e.g., less than four carbons in the main chain) comprising double bonds. While thioesterases have been identified that hydrolyze saturated short carbon chains, it would not have been expected that the identified thioesterases would act upon an unsaturated carbon chain. Thioesterases would be expected to exhibit a high degree of substrate specificity with respect to short carbon chains to avoid hydrolysis of acetyl-CoA, which is critical to fatty synthesis. Unexpectedly, thioesterases that hydrolyze CoA intermediates attached to short, unsaturated carbon chains were identified and successfully produced (or overproduced) in host cells.

Exemplary thioesterases include, but are not limited to, TesB from E. coli and homologs thereof from different organisms. In this regard, the host cell optionally comprises a polynucleotide comprising a nucleic acid sequence encoding an amino acid sequence at least 80% identical (e.g., 85%, 90%, 95%, 99%, or 100% identical) to the amino acid sequence set forth in SEQ ID NO: 90 (TesB), and encoding a polypeptide having thioesterase activity (i.e., the polypeptide hydrolyzes thioesters bonds). An exemplary DNA sequence encoding the TesB amino acid sequence is set out in SEQ ID NO: 89. The amino acid sequences of other known thioesterases are set out in SEQ ID NO: 96, 98, 100, 102, 104, 106 and 108. Exemplary codon-optimized (for E. coli) DNA sequences encoding the thioesterases are respectively set out in SEQ ID NOs: 95, 97, 99, 101, 103, 105 and 107.

Engineered thioesterases also are appropriate for use in the invention. For example, mutation(s) within the active site of a CoA transferase confers thioesterase activity to the enzyme while substantially reducing (if not eliminating) transferase activity. Use of a thioesterase is, in various aspects, superior to use of a CoA transferase by releasing energy associated with the CoA bond. The energy release drives the acrylic acid or crotonic acid pathway to completion. An exemplary method of modifying a CoA transferase to obtain thioesterase activity comprises substituting the amino acid serving as the catalytic carboxylate with an alternate amino acid. CoA transferases suitable for modification and use in the context of the invention include, but are not limited to, acetyl-CoA transferases, propionyl-CoA transferases, and butyryl-CoA transferases. In one aspect, the thioesterase of the invention comprises the amino acid sequence of a propionyl-CoA transferase wherein the catalytic glutamate residue is replaced with an alternate amino acid, such as aspartate. Exemplary propionyl-CoA transferases suitable for mutation include propionyl-CoA transferases from C. propionicum and M. elsdenii. Glutamate residue 324 and glutamate residue 325 are the catalytic carboxylates in C. propionicum propionyl-CoA transferase and M. elsdenii propionyl-CoA transferase, respectively. As the catalytic carboxylate is conserved among CoA transferases, the catalytic amino acid residue in propionate CoA transferases from other sources is identified by sequence alignment with, e.g., the amino acid sequence of C. propionicum propionyl-CoA transferase. Similarly, the catalytic amino acid residue in other CoA transferases (e.g., acetyl-CoA transferase or butyryl-CoA transferases) is identified by sequence alignment with, e.g., the amino acid sequence of C. propionicum propionyl-CoA transferase. C. propionicum propionyl-CoA transferase is an example of a sequence suitable for comparison with other CoA transferases; it will be appreciated that sequences of other CoA transferase sequences can be compared to identify the conserved glutamate catalytic residue for mutation. It will also be appreciated that mutated CoA transferase having thioesterase activity can be generated by altering the nucleic acid sequence of an existing CoA transferase-encoding polynucleotide, or by generating a new polynucleotide based on the coding sequence of a CoA transferase. Thus, in these embodiments, the host cell of the invention comprises a polynucleotide comprising a nucleic acid sequence encoding an amino acid sequence at least 80% identical (e.g., 85%, 90%, 95%, 99%, or 100% identical) to the amino acid sequence set forth in SEQ ID NO: 92 (C. propionicum-derived thioesterase including an E324D substitution) or SEQ ID NO: 94 (M. elsdenii-derived thioesterase including an E325D substitution) and encoding a polypeptide having thioesterase activity. Exemplary codon-optimized (for E. coli) DNA sequences encoding the two thioesterases are respectively set out in SEQ ID NOs: 91 and 93. Amino acid sequences of other engineered thioesterases are set out in SEQ ID NOs: 109, 110, 111 and 112.

### Isolated Enzymes

In some embodiments, isolated enzymes can be used to catalyze one or more steps described in the aspects of the invention. Advantages may include higher product yields, easier product recovery from a more concentrated solution without cell related impurities, a greater range of possible reaction conditions the use of less expensive reactors.

### BRIEF DESCRIPTION OF THE DRAWING

FIG. 1 shows steps in the conversion of glucose to homoserine.

FIG. 2 shows steps in methods of the invention for producing acrylate, 3-hydroxypropionyl-CoA, 3-hydroxypropionate and poly-3-hydroxypropionate from homoserine.

FIG. 3 shows steps in methods of the invention for producing acrylate, 3-hydroxypropionate, 1,3-propanediol and 3-hydroxypropionyl-CoA from homoserine.

FIG. 4 shows single ion monitoring (SIM) LC-MS chromatograms of 2-keto-4-hydroxybutyrate and glutamate, after incubation of L-homoserine and α-ketoglutarate with (reaction) or without (control) Pf_AT aminotransferase.

FIG. 5 show initial rates of deamination as a function of L-homoserine concentration by Pf_AT aminotransferase.

FIG. 6 shows the production of 3-hydroxypropionyl-CoA from L-homoserine catalyzed by D-amino acid oxidase and 2-ketoglutarate dehydrogenase or D-amino acid oxidase, KdcA decarboxylase, and PduP dehydrogenase.

FIG. 7 shows HPLC chromatograms of samples of acryloyl-CoA after incubation with (top) or without (bottom) a dehydratase, evidencing the formation of 3-hydroxypropionyl-CoA only when the enzyme was present.

FIG. 8 shows the production 3-hydroxypropionyl-CoA from acryloyl-CoA catalyzed by a dehydratase.

FIG. 9 shows the consumption of 3-hydroxypropionyl-CoA after incubation with PHA synthase suggesting the formation of the poly(3-hydroxypropionate).

FIG. 10 shows thioesterase activity against an acryloyl-CoA substrate. Activity is monitored by optical density (OD) at 412 nm.

FIG. 11 shows thioesterase activity against an octanoyl-CoA substrate. Activity is monitored by optical density (OD) at 412 nm.

FIG. 12 shows thioesterase activity against an acryloyl-CoA substrate. Activity is monitored by optical density (OD) at 412 nm.

FIG. 13 shows thioesterase activity against an acryloyl-CoA substrate. Activity is monitored by optical density (OD) at 412 nm.

FIG. 14 shows thioesterase activity against an octanoyl-CoA substrate. Activity is monitored by optical density (OD) at 412 nm.

FIG. 15 shows thioesterase activity against an octanoyl-CoA substrate. Activity is monitored by optical density (OD) at 412 nm.

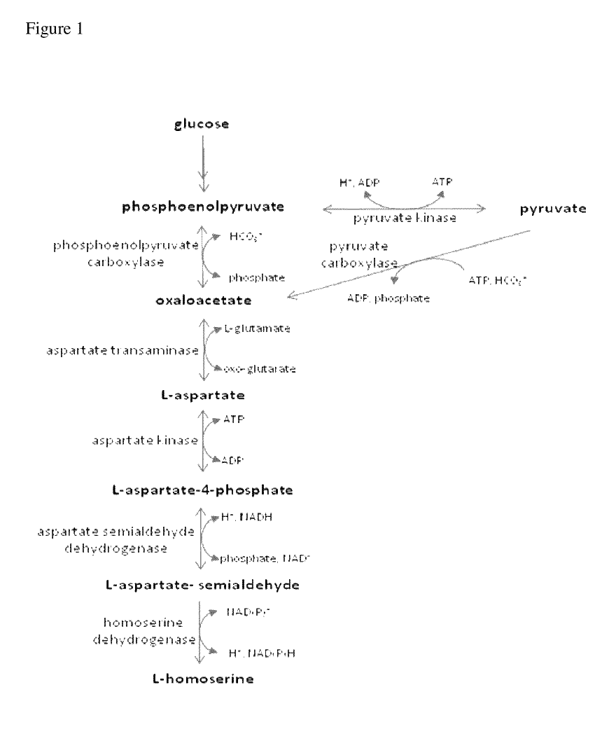

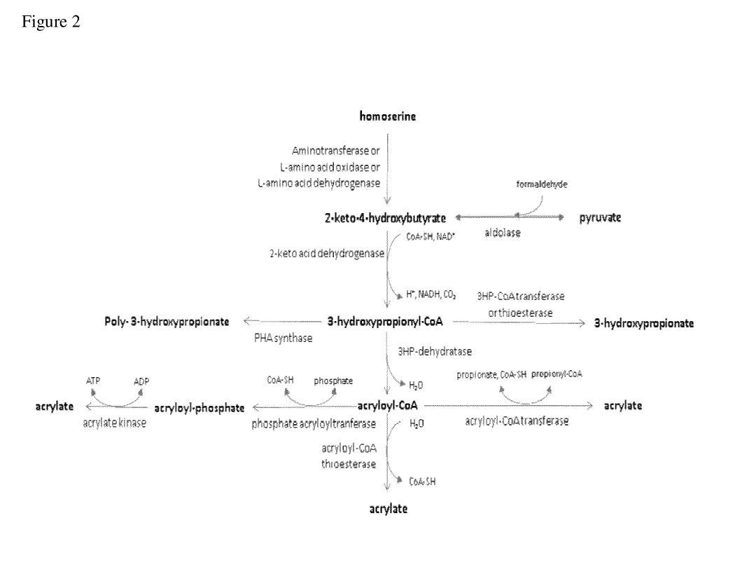

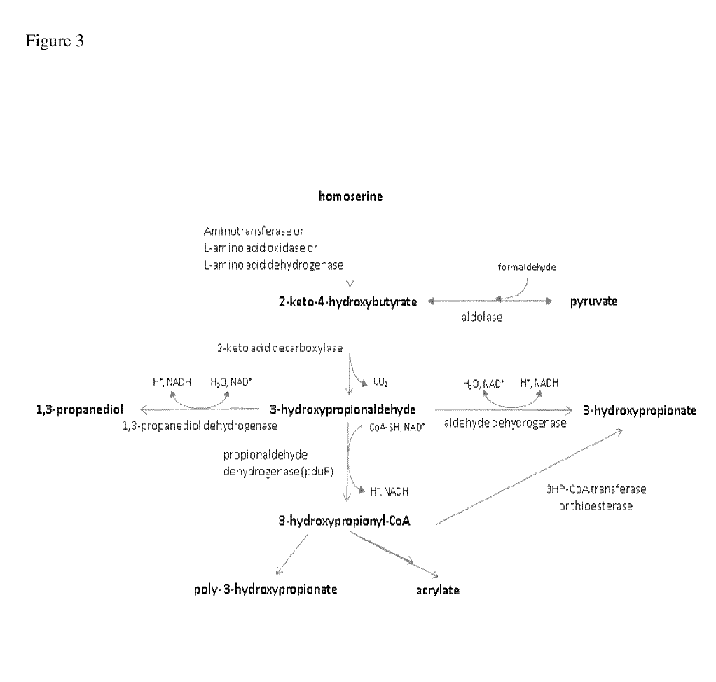

### DETAILED DESCRIPTION OF THE INVENTION

### Definitions

The invention provides the products acrylic acid and acrylate. As is understood in the art, acrylate is the carboxylate anion (i.e., conjugate base) of acrylic acid. The pH of the product solution determines the relative amount of acrylate versus acrylic in a preparation according to the Henderson-Hasselbalch equation {pH=pKa+log([A⁻]/[HA]}, where pKa is −log(Ka). Ka is the acid dissociation constant of acrylic acid. The pKa of acrylic acid in water is about 4.35. Thus, at or near neutral pH, acrylic acid will exist primarily as the carboxylate anion. As used herein, “acrylic acid” and “acrylate” are both meant to encompass the other.

As used herein, “amplify,” “amplified,” or “amplification” refers to any process or protocol for copying a polynucleotide sequence into a larger number of polynucleotide molecules, e.g., by reverse transcription, polymerase chain reaction, and ligase chain reaction.

As used herein, an “antisense sequence” refers to a sequence that specifically hybridizes with a second polynucleotide sequence. For instance, an antisense sequence is a DNA sequence that is inverted relative to its normal orientation for transcription. Antisense sequences can express an RNA transcript that is complementary to a target mRNA molecule expressed within the host cell (e.g., it can hybridize to target mRNA molecule through Watson-Crick base pairing).

As used herein, “cDNA” refers to a DNA that is complementary or identical to an mRNA, in either single stranded or double stranded form.

As used herein, “complementary” refers to a polynucleotide that base pairs with a second polynucleotide. Put another way, “complementary” describes the relationship between two single-stranded nucleic acid sequences that anneal by base-pairing. For example, a polynucleotide having the sequence 5′-GTCCGA-3′ is complementary to a polynucleotide with the sequence 5′-TCGGAC-3′.

As used herein, a “conservative substitution” refers to the substitution in a polypeptide of an amino acid with a functionally similar amino acid. Put another way, a conservative substitution involves replacement of an amino acid residue with an amino acid residue having a similar side chain. Families of amino acid residues having similar side chains have been defined within the art, and include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine).

As used herein, a “corresponding wild-type microorganism” is the naturally-occurring microorganism that would be the same as the microorganism of the invention except that the naturally-occurring microorganism has not been genetically engineered to express any recombinant genes.

As used herein, “encoding” refers to the inherent property of nucleotides to serve as templates for synthesis of other polymers and macromolecules. Unless otherwise specified, a “nucleotide sequence encoding an amino acid sequence” includes all nucleotide sequences that are degenerate versions of each other and that encode the same amino acid sequence.

As used herein, “endogenous” refers to polynucleotides, polypeptides, or other compounds that are expressed naturally or originate within an organism or cell. That is, endogenous polynucleotides, polypeptides, or other compounds are not exogenous. For instance, an “endogenous” polynucleotide or peptide is present in the cell when the cell was originally isolated from nature.

As used herein, “expression vector” refers to a vector comprising a recombinant polynucleotide comprising expression control sequences operatively linked to a nucleotide sequence to be expressed. For example, suitable expression vectors can be an autonomously replicating plasmid or integrated into the chromosome.

As used herein, “exogenous” refers to any polynucleotide or polypeptide that is not naturally found or expressed in the particular cell or organism where expression is desired. Exogenous polynucleotides, polypeptides, or other compounds are not endogenous.

As used herein “homoserine” includes enantiomers such as L-homoserine and D-homoserine.

As used herein, “hybridization” includes any process by which a strand of a nucleic acid joins with a complementary nucleic acid strand through base-pairing. Thus, the term refers to the ability of the complement of the target sequence to bind to a test (i.e., target) sequence, or vice-versa.

As used herein, “hybridization conditions” are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm−5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes. For example, 6×SSC=very low stringency; 3×SSC=low to medium stringency; 1×SSC=medium stringency; and 0.5×SSC=high stringency. Functionally, maximum stringency conditions may be used to identify nucleic acid sequences having strict (i.e., about 100%) identity or near-strict identity with the hybridization probe; while high stringency conditions are used to identify nucleic acid sequences having about 80% or more sequence identity with the probe.

As used herein, “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

“Microorganisms” of the invention expressing recombinant genes are not naturally-occurring. In other words, the microorganisms are man-made and have been genetically engineered to express recombinant genes. The microorganisms of the invention have been genetically engineered to express the recombinant genes encoding the enzymes necessary to carry out the conversion of homoserine to the desired product. Microorganisms of the invention are bacteria, yeast, fungi or algae. Bacteria include, but not limited to, E. coli strains K, B or C. Microorganisms that are more resistant to toxicity of the products of the invention are preferred. Plant cells that are not naturally-occurring (are man-made) and have been genetically engineered to express recombinant genes carrying out the conversions detailed herein are contemplated by the invention to be alternative cells to microorganisms, for example in the production of poly-3-hydroxypropionate.

As used herein, “naturally-occurring” refers to an object that can be found in nature. For example, a polypeptide or polynucleotide sequence that is present in an organism (including viruses) that can be isolated from a source in nature and which has not been intentionally modified by man in the laboratory is naturally-occurring. As used herein, “naturally-occurring” and “wild-type” are synonyms.

As used herein, “operably linked,” when describing the relationship between two DNA regions or two polypeptide regions, means that the regions are functionally related to each other. For example, a promoter is operably linked to a coding sequence if it controls the transcription of the sequence; a ribosome binding site is operably linked to a coding sequence if it is positioned so as to permit translation; and a sequence is operably linked to a peptide if it functions as a signal sequence, such as by participating in the secretion of the mature form of the protein.

As used herein, a recombinant gene that is “over-expressed” produces more RNA and/or protein than a corresponding naturally-occurring gene in the microorganism. Methods of measuring amounts of RNA and protein are known in the art. Over-expression can also be determined by measuring protein activity such as enzyme activity. Depending on the embodiment of the invention, “over-expression” is an amount at least 3%, at least 5%, at least 10%, at least 20%, at least 25%, or at least 50% more. An over-expressed polynucleotide is generally a polynucleotide native to the host cell, the product of which is generated in a greater amount than that normally found in the host cell. Over-expression is achieved by, for instance and without limitation, operably linking the polynucleotide to a different promoter than the polynucleotide's native promoter or introducing additional copies of the polynucleotide into the host cell.

As used herein, “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T.” Put another way, “polynucleotide” refers to a polymer of nucleotides removed from other nucleotides (a separate fragment or entity) or can be a component or element of a larger nucleotide construct, such as an expression vector or a polycistronic sequence. Polynucleotides include DNA, RNA and cDNA sequences.

As used herein, “polypeptide” refers to a polymer composed of amino acid residues which may or may not contain modifications such as phosphates and formyl groups.

As used herein, “primer” refers to a polynucleotide that is capable of specifically hybridizing to a designated polynucleotide template and providing a point of initiation for synthesis of a complementary polynucleotide when the polynucleotide primer is placed under conditions in which synthesis is induced.

As used herein, “recombinant polynucleotide” refers to a polynucleotide having sequences that are not joined together in nature. A recombinant polynucleotide may be included in a suitable vector, and the vector can be used to transform a suitable host cell. A host cell that comprises the recombinant polynucleotide is referred to as a “recombinant host cell.” The polynucleotide is then expressed in the recombinant host cell to produce, e.g., a “recombinant polypeptide.”

As used herein, “recombinant expression vector” refers to a DNA construct used to express a polynucleotide that, e.g., encodes a desired polypeptide. A recombinant expression vector can include, for example, a transcriptional subunit comprising (i) an assembly of genetic elements having a regulatory role in gene expression, for example, promoters and enhancers, (ii) a structural or coding sequence which is transcribed into mRNA and translated into protein, and (iii) appropriate transcription and translation initiation and termination sequences. Recombinant expression vectors are constructed in any suitable manner. The nature of the vector is not critical, and any vector may be used, including plasmid, virus, bacteriophage, and transposon. Possible vectors for use in the invention include, but are not limited to, chromosomal, nonchromosomal and synthetic DNA sequences, e.g., bacterial plasmids; phage DNA; yeast plasmids; and vectors derived from combinations of plasmids and phage DNA, DNA from viruses such as vaccinia, adenovirus, fowl pox, baculovirus, SV40, and pseudorabies.

As used herein, a “recombinant gene” is not a naturally-occurring gene. A recombinant gene is man-made. A recombinant gene includes a protein coding sequence operably linked to expression control sequences. Embodiments include, but are not limited to, an exogenous gene introduced into a microorganism, an endogenous protein coding sequence operably linked to a heterologous promoter (i.e., a promoter not naturally linked to the protein coding sequence) and a gene with a modified protein coding sequence (e.g., a protein coding sequence encoding an amino acid change or a protein coding sequence optimized for expression in the microorganism). The recombinant gene is maintained in the genome of the microorganism, on a plasmid in the microorganism or on a phage in the microorganism.

As used herein, “reduced” expression is expression of less RNA or protein than the corresponding natural level of expression. Methods of measuring amounts of RNA and protein are known in the art. Reduced expression can also be determined by measuring protein activity such as enzyme activity. Depending on the embodiment of the invention, “reduced” is an amount at least 3%, at least 5%, at least 10%, at least 20%, at least 25%, or at least 50% less.

As used herein, “specific hybridization” refers to the binding, duplexing, or hybridizing of a polynucleotide preferentially to a particular nucleotide sequence under stringent conditions.

As used herein, “stringent conditions” refers to conditions under which a probe will hybridize preferentially to its target subsequence, and to a lesser extent to, or not at all to, other sequences.

As used herein, “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

### Polynucleotides

The polynucleotide(s) encoding one or more enzyme activities for steps in the pathways of the invention may be derived from any source. Depending on the embodiment of the invention, the polynucleotide is isolated from a natural source such as bacteria, algae, fungi, plants, or animals; produced via a semi-synthetic route (e.g., the nucleic acid sequence of a polynucleotide is codon optimized for expression in a particular host cell, such as E. coli); or synthesized de novo. In certain embodiments, it is advantageous to select an enzyme from a particular source based on, e.g., the substrate specificity of the enzyme or the level of enzyme activity in a given host cell. In some embodiments of the invention, the enzyme and corresponding polynucleotide are naturally found in the host cell and over-expression of the polynucleotide is desired. In this regard, in some embodiments, additional copies of the polynucleotide are introduced in the host cell to increase the amount of enzyme. In some embodiments, over-expression of an endogenous polynucleotide may be achieved by upregulating endogenous promoter activity, or operably linking the polynucleotide to a more robust heterologous promoter.

Exogenous enzymes and their corresponding polynucleotides also are suitable for use in the context of the invention, and the features of the biosynthesis pathway or end product can be tailored depending on the particular enzyme used.

The invention contemplates that polynucleotides of the invention may be engineered to include alternative degenerate codons to optimize expression of the polynucleotide in a particular microorganism. For example, a polynucleotide may be engineered to include codons preferred in E. coli if the DNA sequence will be expressed in E. coli. Methods for codon-optimization are known in the art.

### Enzyme Variants

In certain embodiments, the microorganism produces an analog or variant of the polypeptide encoding an enzyme activity. Amino acid sequence variants of the polypeptide include substitution, insertion, or deletion variants, and variants may be substantially homologous or substantially identical to the unmodified polypeptides. In certain embodiments, the variants retain at least some of the biological activity, e.g., catalytic activity, of the polypeptide. Other variants include variants of the polypeptide that retain at least about 50%, preferably at least about 75%, more preferably at least about 90%, of the biological activity.

Substitutional variants typically exchange one amino acid for another at one or more sites within the protein. Substitutions of this kind can be conservative, that is, one amino acid is replaced with one of similar shape and charge. Conservative substitutions include, for example, the changes of: alanine to serine; arginine to lysine; asparagine to glutamine; aspartate to glutamate; cysteine to serine; glutamine to asparagine; glutamate to aspartate; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. An example of the nomenclature used herein to indicate a amino acid substitution is “S345F ThrA” wherein the naturally occurring serine occurring at position 345 of the naturally occurring ThrA enzyme which has been substituted with a phenylalanine.

In some instances, the microorganism comprises an analog or variant of the exogenous or over-expressed polynucleotide(s) described herein. Nucleic acid sequence variants include one or more substitutions, insertions, or deletions, and variants may be substantially homologous or substantially identical to the unmodified polynucleotide. Polynucleotide variants or analogs encode mutant enzymes having at least partial activity of the unmodified enzyme. Alternatively, polynucleotide variants or analogs encode the same amino acid sequence as the unmodified polynucleotide. Codon optimized sequences, for example, generally encode the same amino acid sequence as the parent/native sequence but contain codons that are preferentially expressed in a particular host organism.

A polypeptide or polynucleotide “derived from” an organism contains one or more modifications to the naturally-occurring amino acid sequence or nucleotide sequence and exhibits similar, if not better, activity compared to the native enzyme (e.g., at least 70%, at least 80%, at least 90%, at least 95%, at least 100%, or at least 110% the level of activity of the native enzyme). For example, enzyme activity is improved in some contexts by directed evolution of a parent/naturally-occurring sequence. Additionally or alternatively, an enzyme coding sequence is mutated to achieve feedback resistance.

Expression Vectors/Transfer into Microorganisms

Expression vectors for recombinant genes can be produced in any suitable manner to establish expression of the genes in a microorganism. Expression vectors include, but are not limited to, plasmids and phage. The expression vector can include the exogenous polynucleotide operably linked to expression elements, such as, for example, promoters, enhancers, ribosome binding sites, operators and activating sequences. Such expression elements may be regulatable, for example, inducible (via the addition of an inducer). Alternatively or in addition, the expression vector can include additional copies of a polynucleotide encoding a native gene product operably linked to expression elements. Representative examples of useful heterologous promoters include, but are not limited to: the LTR (long terminal 35 repeat from a retrovirus) or SV40 promoter, the E. coli lac, tet, or trp promoter, the phage Lambda PL promoter, and other promoters known to control expression of genes in prokaryotic or eukaryotic cells or their viruses. In one aspect, the expression vector also includes appropriate sequences for amplifying expression. The expression vector can comprise elements to facilitate incorporation of polynucleotides into the cellular genome.

Introduction of the expression vector or other polynucleotides into cells can be performed using any suitable method, such as, for example, transformation, electroporation, microinjection, microprojectile bombardment, calcium phosphate precipitation, modified calcium phosphate precipitation, cationic lipid treatment, photoporation, fusion methodologies, receptor mediated transfer, or polybrene precipitation. Alternatively, the expression vector or other polynucleotides can be introduced by infection with a viral vector, by conjugation, by transduction, or by other suitable methods.

### Culture

Microorganisms of the invention comprising recombinant genes are cultured under conditions appropriate for growth of the cells and expression of the gene(s). Microorganisms expressing the polypeptide(s) can be identified by any suitable methods, such as, for example, by PCR screening, screening by Southern blot analysis, or screening for the expression of the protein. In some embodiments, microorganisms that contain the polynucleotide can be selected by including a selectable marker in the DNA construct, with subsequent culturing of microorganisms containing a selectable marker gene, under conditions appropriate for survival of only those cells that express the selectable marker gene. The introduced DNA construct can be further amplified by culturing genetically modified microorganisms under appropriate conditions (e.g., culturing genetically modified microorganisms containing an amplifiable marker gene in the presence of a concentration of a drug at which only microorganisms containing multiple copies of the amplifiable marker gene can survive).

In some embodiments, the microorganisms (such as genetically modified bacterial cells) have an optimal temperature for growth, such as, for example, a lower temperature than normally encountered for growth and/or fermentation. In addition, in certain embodiments, cells of the invention exhibit a decline in growth at higher temperatures as compared to normal growth and/or fermentation temperatures as typically found in cells of the type.

Any cell culture condition appropriate for growing a microorganism and synthesizing a product of interest is suitable for use in the inventive method.

### Recovery

The methods of the invention optionally comprise a step of product recovery. Recovery of acrylate, 3-hydroxypropionyl-CoA, 3-hydroxypropionate, poly-3-hydroxypropionate or 1,3-propanediol can be carried out by methods known in the art. For example, acrylate can be recovered by distillation methods, extraction methods, crystallization methods, or combinations thereof; 3-hydroxypropionate can be recovered as described in U.S. Published Patent Application No. 2011/038364 or International Publication No. WO 2011/0125118; polyhydroxyalkanoates can be recovered as described in Yu and Chen, Biotechnol Prog, 22(2): 547-553 (2006); and 1,3 propanediol can be recovered as described in U.S. Pat. No. 6,428,992 or Cho et al., Process Biotechnology, 41(3): 739-744 (2006).

### EXAMPLES

The following examples further describe and demonstrate embodiments within the scope of the present invention. The examples are given solely for the purpose of illustration and are not to be construed as limiting the present invention. Examples 1 to 6 describe the construction of different plasmids for heterologous expression of proteins in E. coli; Examples 7 and 8 describe the transformation and culture of E. coli strains; Examples 9 and 10 describe the purification of several proteins; Example 12 describes a method for quantification of acyl-CoA molecules; Examples 11 and 13 to 16 describe the in vitro reconstitution of the enzymatic activity of several proteins described in the present invention; Example 17 describe the production of 3-hydroxypropionic acid in engineered E. coli.

### Example 1

### Expression Vectors for Aminotransferase Genes

E. coli expression vectors were constructed for production of recombinant aminotransferases. A common cloning strategy was established utilizing the pET30a vector (Novagen, EMD Chemicals, Gibbstown, N.J., catalog #69909-30) for expression of proteins linked to an N-terminal hexahistidine tag under the T7 promoter. Modifications to the pET30a vector were made by replacing the DNA sequence between the SphI and XhoI sites with a synthesized DNA sequence (SEQ ID NO: 117) (GenScript, Piscataway, N.J.). In this resulting vector, designated pET30a-BB, the XbaI site in the lac operator was removed and the region encoding for the thrombin, S-tag and enterokinase sites was replaced for a sequence encoding for a Factor Xa recognition site. Furthermore, the multiple cloning site was modified to include EcoRV, EcoRI, BamHI, SacI, and PstI sites.

Several aminotransferase genes were codon-optimized for expression in E. coli. To facilitate cloning, the common restriction sites: AvrII; BamHI; BglII; BstBI; EagI; EcoRI; EcoRV; HindIII; KpnI; NcoI; NheI; NotI; NspV; PstI; PvuII; SacI; SalI; SapI; Sful; SpeI; XbaI; XhoI were also removed from the gene sequences. In addition, the 5′ prefix sequence (SEQ ID NO: 118) was added immediately upstream of the start codon and a SpeI, NotI and PstI restriction site 3′ suffix sequence (SEQ ID NO: 119) was added immediately downstream of the stop codon. The optimized sequences were synthesized (GenScript, Piscataway, N.J.) and cloned into the pET30a-BB vector at the KpnI and PstI sites. The resulting plasmids and the encoded proteins are described in Table 1.

TABLE 1
List of plasmids encoding for different aminotransferases

| | | | Accession# |
|---|---|---|---|
| | Enzyme | | (Amino Acid |
| Plasmid | Key | Species and Protein (DNA SEQ ID NO:) | SEQ ID NO:) |
| pET30a-BB Pf AT | Pf AT | Pseudomonas fluorescens branched-chain | YP_002873519.1 |
| | | amino acid aminotransferase (SEQ ID NO: | (SEQ ID NO: 8) |
| | | 122) | |
| pET30a-BB Ec AT | Ec AT | E. coli valine-pyruvate aminotransferase (SEQ | NP_416793.1 |
| | | ID NO: 123) | (SEQ ID NO: 9) |
| pET30a-BB Rn AT | Rn AT | Rattus norvegicus Alanine aminotransferase | BAA01185.1 |
| | | (SEQ ID NO: 121) | (SEQ ID NO: 4) |
| pET30a-BB Ss AT | Ss AT | Sus scrofa aspartate aminotransferase, | NP_999092.1 |
| | | cytoplasmic (SEQ ID NO: 120) | (SEQ ID NO: 2) |

### Example 2

### Expression Vector for Branched-Chain 2-Keto Acid Decarboxylase (KdcA)

An E. coli expression vector was constructed for production of a recombinant branched-chain 2-keto acid decarboxylase (KdcA). A Lactococcus lactis branched-chain 2-keto acid decarboxylase gene was codon-optimized for expression in E. coli, and the common restriction sites: AvrII; BamHI; BglII; BstBI; EagI; EcoRI; EcoRV; HindIII; KpnI; NcoI; NheI; NotI; NspV; PstI; PvuII; SacI; SalI; SapI; SfuI; SpeI; XbaI; XhoI were removed to facilitate cloning. Furthermore, additional EcoRI, NotI, XbaI restriction sites and a ribosomal binding site (RBS) 5′ to the ATG start codon, and SpeI, NotI and PstI restriction sites 3′ to the stop codon were included into the sequence. The optimized sequence (SEQ ID NO: 124) was synthesized (GenScript, Piscataway, N.J.) and cloned into the pET30a-BB vector at the EcoRI and PstI sites. The resulting expression vector encoding N-terminal histidine tagged KdcA (SEQ ID NO: 54) was designated pET30a-BB Ll KDCA.

### Example 3

### Expression Vector for Coenzyme-A Acylating Propionaldehyde Dehydrogenase (PduP)

An E. coli expression vector was constructed for production of a recombinant coenzyme-A acylating propionaldehyde dehydrogenase (PduP). A Salmonella enterica coenzyme-A acylating propionaldehyde dehydrogenase gene was codon-optimized for expression in E. coli, and the common restriction sites: AvrII; BamHI; BglII; BstBI; EagI; EcoRI; EcoRV; HindIII; KpnI; NcoI; NheI; NotI; NspV; PstI; PvuII; SacI; SalI; SapI; SfuI; SpeI; XbaI; XhoI were removed to facilitate cloning. Furthermore, additional EcoRI, NotI, XbaI restriction sites and a ribosomal binding site (RBS) 5′ to the ATG start codon, and SpeI, NotI and PstI restriction sites 3′ to the stop codon were included into the sequence. The optimized sequence (SEQ ID NO: 125) was synthesized (GenScript, Piscataway, N.J.) and cloned into the pET30a-BB vector at the EcoRI and PstI sites. The resulting expression vector, designated pET30a-BB Se PDUP, encodes N-terminal histidine tagged version of PduP (SEQ ID NO: 60).

### Example 4

### Expression Vector for poly(3-hydroxybutyrate) Polymerase (PhaC or PHA Synthase)

An E. coli expression vector was constructed for production of a recombinant poly(3-hydroxybutyrate) polymerase. A Cupriavidus necator poly (3-hydroxybutyrate) polymerase (phaC) gene was codon-optimized for expression in E. coli, and the common restriction sites: AvrII; BamHI; BglII; BstBI; EagI; EcoRI; EcoRV; HindIII; KpnI; NcoI; NheI; NotI; NspV; PstI; PvuII; Sad; SalI; SapI; Sful; SpeI; XbaI; XhoI were removed to facilitate cloning. Furthermore, additional EcoRI, NotI, XbaI restriction sites and a ribosomal binding site (RBS) 5′ to the ATG start codon, and SpeI, NotI and PstI restriction sites 3′ to the stop codon were included into the sequence. The optimized sequence (SEQ ID NO: 126) was synthesized (GenScript, Piscataway, N.J.) and cloned into the pET30a-BB vector at the EcoRI and PstI sites. The resulting expression vector, designated pET30a-BB Cn PHAS, encodes N-terminal histidine tagged version of PHA synthase (SEQ ID NO: 42).

### Example 5

### Expression Vector for 3-hydroxypropionyl-CoA dehydratase

An E. coli expression vector was constructed for production of a recombinant 3-hydroxypropionyl-CoA dehydratase. A Metallosphaera sedula 3-hydroxypropionyl-CoA dehydratase gene was codon-optimized for expression in E. coli, and the common restriction sites: AvrII; BamHI; BglII; BstBI; EagI; EcoRI; EcoRV; HindIII; KpnI; NcoI; NheI; NotI; NspV; PstI; PvuII; Sad; SalI; SapI; SfuI; SpeI; XbaI; XhoI were removed to facilitate cloning. Furthermore, additional EcoRI, NotI, XbaI restriction sites and a ribosomal binding site (RBS) 5′ to the ATG start codon, and SpeI, NotI and PstI restriction sites 3′ to the stop codon were included into the sequence. The optimized sequence (SEQ ID NO: 127) was synthesized (GenScript, Piscataway, N.J.) and cloned into the pET30a-BB vector at the EcoRI and PstI sites. The resulting expression vector, designated pET30a-BB Ms 3HP-CD, encodes N-terminal histidine tagged version of the dehydratase (SEQ ID NO: 48).

### Example 6

### Expression Vectors for Acyl-CoA Thioesterase

E. coli expression vectors were constructed for production of recombinant short to medium-chain acyl-CoA thioesterases. Thioesterase genes from different organisms were codon-optimized for expression in E. coli, and the common restriction sites: BamHI, BglII, BstBI, EcoRI, HindIII, KpnI, PstI, NcoI, NotI, SacI, SalI, XbaI, and XhoI were removed to facilitate cloning. Furthermore, additional BamHI and XbaI restriction sites 5′ to the ATG start codon, and SacI and HindIII restriction sites 3′ to the stop codon were included into the sequence. The optimized sequences were synthesized (GenScript, Piscataway, N.J. or GeneArt, Invitrogen, Carlsbad, Calif.) and cloned into the pET30a vector at the BamHI and Sad sites. The resulting plasmids and the encoded proteins are described in Table 2.

TABLE 2
List of plasmids encoding for different thioesterases

| Plasmid | Enzyme Key | Species/Protein | Accession # |
|---|---|---|---|
| pET30a-Sc Acot8 | ScACOT8 | Saccharomyces cerevisiae | NP_012553 |
| | | peroxisomal acyl-CoA thioesterase | |
| | | (SEQ ID NO: 96) | |
| pET30a-Mus Acot8 | MusACOT8 | Mus musculus acyl-CoA thioesterase | AAL35333 |
| | | 8 (SEQ ID NO: 98) | |
| pET30a-Rn Acot12 | RnACOT12 | R. norvegicus acyl-CoA thioesterase | NP_570103 |
| | | 12 (SEQ ID NO: 100) | |
| pET30a-Ec TesB | EcTesB | E. coli acyl-CoA thioesterase II | NP_286194 |
| | | (TesB) (SEQ ID NO: 90) | |
| pET30a-Bs SrfD | BsSrfD | Bacillus subtilis surfactin synthetase | NP_388234 |
| | | (SrfAD) (SEQ ID NO: 102) | |
| pET30a-Cp T | CpT | C. propionicum propionate CoA- | CAB77207 |
| | | transferase | |
| pET30a-Cp TT | CpTT | C. propionicum propionate CoA- | Similar to |
| | | transferase (with E324D mutation) | CAB77207 |
| | | (SEQ ID NO: 92) | |
| pET30a-Me T | MeT | M. elsdenii coenzyme A-transferase | Similar to |
| | | | CCC72964 |
| | | | except for |
| | | | T271A and |
| | | | K517R |
| pET30a-Me TT | MeTT | M. elsdenii coenzyme A-transferase | Similar to |
| | | (with E325D mutation) (SEQ ID | CCC72964 |
| | | NO: 94) | except for |
| | | | T271A, |
| | | | K517R, and |
| | | | E325D |
| pET30a-Hi YbgC | HiYbgC | Haemophilus influenzae thioesterase | YP_248101 |
| | | (YbgC) (SEQ ID NO: 108) | |

### Example 7

### Transformation of E. coli

The recombinant plasmids were then used to transform chemically competent One Shot BL21 (DE3) pLysS E. coli cells (Invitrogen, Carlsbad, Calif.). Individual vials of cells were thawed on ice and gently mixed with 10 ng of plasmid DNA. The vials were incubated on ice for 30 min. The vials were briefly incubated at 42° C. for 30 sec and quickly replaced back on ice for an additional 2 min. An aliquot of 250 μL of 37° C. SOC medium was added and the vials were secured horizontally on a shaking incubator platform and incubated for 1 h at 37° C., 225 rpm. Aliquots of 20 μL and 200 μL cells were plated onto LB agar plates supplemented with the appropriate antiobiotics (50 μg/mL kanamycin; 34 μg/mL chloramphenicol) to select for cells carrying the recombinant and pLysS plasmids respectively, followed by incubation overnight at 37° C. Single colony isolates were isolated, cultured in 5 mL of selective LB broth and recombinant plasmids were isolated using a QIAPrep® Spin Miniprep kit (Qiagen, Valencia, Calif.) spin plasmid miniprep kit. Plasmid DNAs were characterized by gel electrophoresis of restriction digests with AflIII.

### Example 8

### Culture of E. coli Strains and Expression of Recombinant Proteins

Aliquots of LB broth (15 mL), supplemented with the appropriate antibiotics (34 μg/mL chloramphenicol; 50 μg/mL kanamycin) were inoculated with different E. coli strains from frozen glycerol stocks. Cultures were incubated overnight at 25° C. with 250 rpm shaking. LB broth (150 ml, containing 34 μg/mL chloramphenicol, 50 μg/mL kanamycin; equilibrated to 25° C.) in 1 to 2.8 L fluted Erlenmeyer flasks was inoculated from the overnight cultures at an optical density (OD) at 600 nm of ˜0.1. Cultures were continued at 25° C. with 250 rpm shaking and optical density was monitored until A₆₀₀ of ˜0.4. Production of recombinant proteins was induced by addition of 1M IPTG (Teknova, Hollister, Calif.; 1 mM final concentration). Cultures were further incubated for 24 h at 25° C. with 250 rpm shaking before harvesting by centrifugation. The cell pellets were stored at −80° C. until used.

### Example 9

### Recombinant Protein Isolation

His-tagged recombinant proteins were isolated by immobilized metal affinity chromatography (IMAC) utilizing nickel-nitrilotriacetic acid coupled Sepharose CL-6B resin (Ni-NTA, Qiagen, Valencia, Calif.) as follows. Cell pellets were thawed on ice and suspended in 20 mL of binding buffer (20 mM sodium phosphate, 500 mM NaCl, 20 mM imidazole, pH 7.4) supplemented with 1 mg/mL lysozyme and 1 pellet of Complete EDTA-free protease inhibitor (Roche Applied Science, Indianapolis, Ind.). Samples were incubated at 4° C. with 30 rpm rotation for 30 min followed by French-pressing (1000 psi). Cell debris was pelleted by centrifugation for 1 h at 15,000×g and 4° C. The supernatant was transferred to a 5 mL column bed of Ni-NTA resin equilibrated with binding buffer. The Ni-NTA resin was resuspended in the supernatant and incubated for 60 min with slow rocker mixing at 4° C. The unbound material was removed by gravity flow and the resin was washed by gravity flow with 20 column volume (CV, 100 mL) of binding buffer followed by 10 CV (50 mL) of rinse buffer (20 mM sodium phosphate, 500 mM NaCl, 100 mM imidazole, pH 7.4). Bound proteins were eluted by gravity-flow in 10 CV (50 mL) of elution buffer (20 mM sodium phosphate, 500 mM NaCl, 500 mM imidazole, pH 7.4) and collected in fractions. Elution aliquots were assayed for protein content by SDS-PAGE analysis, pooled, and concentrated with Amicon Ultra-15 centrifugal filter devices (EMD Millipore, Billerica, Mass.) with 30 kDa nominal molecular weight cut-off. The concentrated protein isolates were desalted and eluted into 3.5 mL of storage buffer (50 mM HEPES, 300 mM NaCl, 20% glycerol, pH 7.3) using PD-10 desalting columns (GE Healthcare Biosciences, Pittsburgh, Pa.).

### Example 10

### Recombinant Thioesterases Isolation

His-tagged recombinant thioesterases were isolated by IMAC utilizing sepharose based magnetic beads with nickel ions (His Mag Sepharose Ni) as follows. Cell pellets were thawed at room temperature and suspended in 1.7 mL of 1× BugBuster (primary amine free; with 10/mL Benzonase nuclease; Novagen #70923-3 and 70750-3 respectively). Samples were incubated at room temperature with 60 rpm rotation for 30 min. Cell debris was pelleted by centrifugation for 10 min at 14,000 rpm. The supernatants were transferred to His Mag Sepharose Ni (GE Healthcare Biosciences, Piscataway, N.J. #28-9799-17) beads equilibrated according to kit instructions in binding buffer (20 mM sodium phosphate, 500 mM NaCl, 20 mM imidazole, pH 7.4). The beads were suspended in the supernatant and incubated for 60 min with slow end-over-end mixing. The beads were then washed for a total of 5 times in 800 μl of binding buffer and slow end-over-end mixing for ˜3-5 min with each wash. The recombinant thioesterases were eluted from the beads in 300 μL of elution buffer (20 mM sodium phosphate, 500 mM NaCl, 500 mM imidazole, pH 7.4) by slow end-over-end mixing for 5 min.

### Example 11

### In Vitro Reconsitution of Aminotransferases and Liquid Chromatography Coupled to Mass Spectrometry (LC-MS) Analysis

The activities of purified recombinant aminotransferases (Example 9) were tested by LC-MS analysis of expected products. In separate reactions, each enzyme was added at 0.27 mg/mL final concentration to reaction buffer (20 mM potassium phosphate, 500 mM sodium chloride, pH 8). L-Homoserine (Sigma, St. Louis, Mo.; catalog #H6515) or a different amino acid substrate (Sigma, St. Louis, Mo.) was added at 1 mM final concentration. Secondary substrates were either α-ketoglutaric acid (disodium salt, dehydrate; Sigma, St. Louis, Mo.; catalog #75892) or pyruvate (Sigma, St. Louis, Mo.; catalog #P2256), each at 1 mM final concentration. Pyroxidal 5′-phosphate hydrate (Sigma, St. Louis, Mo.; catalog #P9255) was added at 50 μM final concentration. The reactions were incubated overnight at room temperature. After incubation, each solution was filtered using Amicon Ultra centrifugal filter devices (EMD Millipore, Billerica, Mass.) with 3 kDa nominal molecular weight cut-off that had been prewashed with ultra pure water. The filtrates were collected and stored at −20° C. until LC-MS analysis.

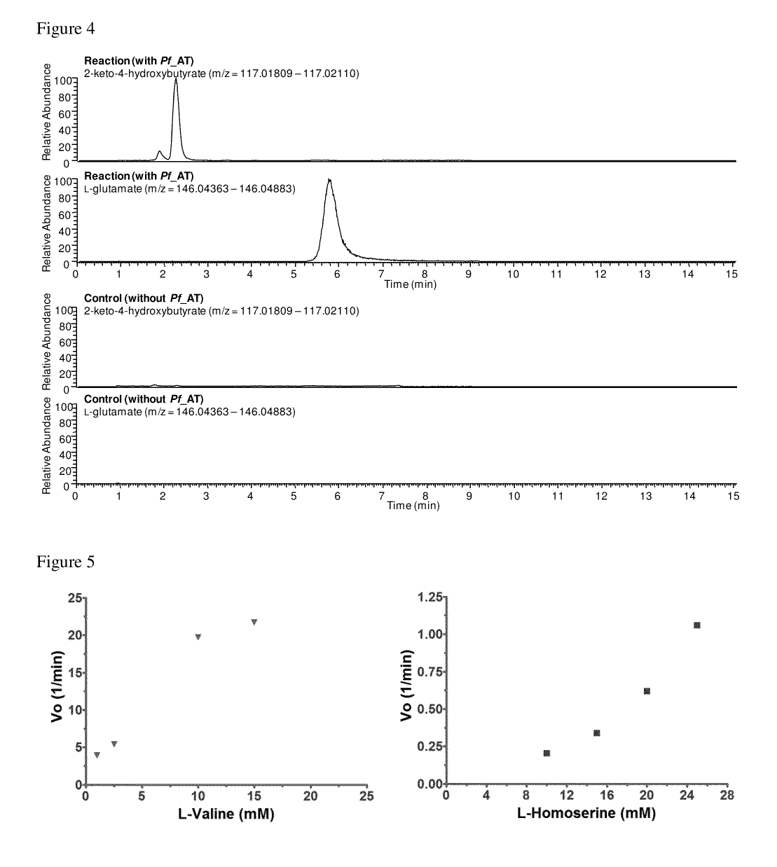

Aliquots of reaction mixture were diluted 50-100× and analyzed by high performance liquid chromatography coupled to mass spectrometry (LC-MS) in negative mode, using an electrospray ionization (ESI) Fourier transform orbital trapping MS (Exactive Model; Thermo Fisher, San Jose, Calif.) at 50,000 resolution. Separations were performed using a ZIC-pHILIC column (2.1×100 mm, 5 μm polymer, Sequant, EMD Millipore, Catalog #1504620001; Darmstadt, Germany) and a mobile phase of 2 mM ammonium formate in 85% acetonitrile/15% water at a flow rate of 200 μL/min. The LC-MS analysis indicated that every tested enzyme (Table 1) produced the expected product when combined with its ideal substrate and all enzymes produced 2-keto-4-hydroxybutyrate when combined with L-homoserine (FIG. 4).

Spectrophotometric Assays with Aminotransferases

To further confirm the enzymatic activity of the aminotransferases, the purified recombinant proteins were assayed spectrophotometrically in a series of coupled enzyme reactions. In separate reactions, Pf AT aminotransferase was added at 0.27 mg/ml final concentration to 100 mM potassium phosphate buffer (pH 8.0; Sigma, St. Louis, Mo.). L-Homoserine (Sigma, St. Louis, Mo.; catalog #H6515) or L-Valine (Sigma, St. Louis, Mo.; catalog #V0500) was added as a substrate at 10-25 mM final concentration. The aminotransferase reaction was coupled with a dehdrogenase reaction in order to generate reduced β-nicotinamide adenine dinucleotide (NADH) which can be detected spectrophotometrically. β-Nicotinamide adenine dinucleotide (NAD⁺; Sigma, St. Louis, Mo.; catalog #N8410) was added at 3 mM final concentration. Pyroxidal 5′-phosphate hydrate (Sigma, St. Louis, Mo.; catalog #P9255) was added at 50 μM final concentration. α-Ketoglutaric acid, disodium salt, dehydrate (Sigma, St. Louis, Mo.; catalog #75892) was added as a secondary substrate at 1 mM final concentration. L-Glutamic dehydrogenase from bovine liver (Sigma, St. Louis, Mo.; catalog #G2626) was added at 10 U/mL. Each reaction was added to a 1 mL quartz cuvette and the formation of NADH was followed over time at 340 nm in a spectrophotometer. As expected, the initial rate of conversion of L-homoserine was dependent on its concentration (FIG. 5). Saturation of the enzyme with L-homoserine was not achieved even when high concentrations were used.

### Example 12

### Acyl-CoA Levels as a Measurement of Enzymatic Activities

### Liquid Chromatography Coupled to Mass Spectrometry (LC-MS)

E. coli Culture Sample Preparation for Acyl-CoA Levels Analysis

A stable-labeled (deuterium) internal standard-containing master mix is prepared, comprising d₃-3-hydroxymethylglutaryl-CoA (200 μL of 50 μg/mL stock in 10 mL of 15% trichloroacetic acid). An aliquot (500 μl) of the master mix is added to a 2-mL tube. Silicone oil (AR200; Sigma, St. Louis, Mo.; catalog #85419; 800 μl) is layered onto the master mix. An aliquot of E. coli culture (800 μl) is layered gently on top of the silicone oil. The sample is subject to centrifugation at 20,000×g for 5 min at 4° C. in an Eppendorf 5417C centrifuge. An aliquot (300 μL) of the master mix-containing layer is transferred to an empty tube and frozen on dry ice for 30 min.

### Measurement of Acyl-CoA Levels

The acyl-CoA content of samples was determined using LC-MS/MS. Individual acyl-CoA standards were purchased from Sigma (St. Louis, Mo.) and prepared as 500 μg/mL stocks in methanol. Acryloyl-CoA was synthesized and prepared similarly. The analytes were pooled, and standards with all of the analytes were prepared by dilution with 15% trichloroacetic acid. Standards for regression were prepared by transferring 500 μL of the working standards to an autosampler vial containing 10 μL of the 50 μg/mL internal standard. Sample peak areas (or heights) were normalized to the stable-labeled internal standard (d₃-3-hydroxymethylglutaryl-CoA). Samples were assayed by LC-MS/MS on a Sciex API5000 mass spectrometer in positive ion Turbo Ion Spray. Separation was carried out by reversed-phase high performance liquid chromatography (HPLC) using a Phenomenex Onyx Monolithic C18 column (2×100 mm) and mobile phases A (5 mM ammonium acetate, 5 mM dimethylbutylamine, and 6.5 mM acetic acid) and B (0.1% formic acid in acetonitrile), with the gradient described in table 3 at a flow rate of 0.6 mL/min.

TABLE 3
Composition of mobile phase during LC-MS/MS analysis

| Time | Mobile Phase A (%) | Mobile Phase B (%) |
|---|---|---|
| 0 min | 97.5 | 2.5 |
| 1.0 min | 97.5 | 2.5 |
| 2.5 min | 91.0 | 9.0 |
| 5.5 min | 45 | 55 |
| 6.0 min | 45 | 55 |
| 6.1 min | 97.5 | 2.5 |
| 7.5 min | — | — |
| 9.5 min | End Run | |

The conditions on the mass spectrometer were: DP 160, CUR 30, GS1 65, GS2 65, IS 4500, CAD 7, TEMP 650 C. The transitions used for the multiple reaction monitoring (MRM) are described in table 4.

TABLE 4
Description of parameters for quantification of different
acyl-CoA molecules

| | Precursor | Product | Collision | |
|---|---|---|---|---|
| Compound | Ion¹ | Ion¹ | Energy | CXP |
| 3-Hydroxypropionyl-CoA² | 840.3 | 333.2 | 45 | 13 |
| n-Propionyl-CoA | 824.3 | 317.2 | 41 | 32 |
| Succinyl-CoA | 868.2 | 361.1 | 49 | 38 |
| Isobutyryl-CoA | 838.3 | 331.2 | 43 | 21 |
| Lactoyl-CoA | 840.3 | 333.2 | 45 | 38 |
| Acryloyl-CoA | 822.4 | 315.4 | 45 | 36 |
| Coenzyme A | 768.3 | 261.2 | 45 | 34 |
| Isovaleryl-CoA | 852.2 | 345.2 | 45 | 34 |
| Malonyl-CoA | 854.2 | 347.2 | 41 | 36 |
| Acetyl-CoA | 810.3 | 303.2 | 43 | 30 |
| d₃-3-Hydroxymethylglutaryl- | 915.2 | 408.2 | 49 | 13 |
| CoA | | | | |

¹Energies, in volts, for the MS/MS analysis
²Quantified based on n-propionyl-CoA response

### Example 13

### In Vitro Production of 3-Hydroxypropionyl-CoA with 2-Keto Acid Decarboxylases or Dehydrogenases

In a first assay, D-homoserine (2 mM; Acros, Geel, Belgium; catalog #348362500) was incubated with D-amino acid oxidase (1 U/mL; Sigma, St. Louis, Mo.; catalog #A5222) and bovine liver catalase (600 U/mL; Sigma, St. Louis, Mo.; catalog #C40) in the presence of HEPES buffer (50 mM, pH 7.3). After incubation at room temperature for 2-4 h, coenzyme A (2 mM), β-NAD⁺ (2 mM), thiamine pyrophosphate (0.2 mM), and MgCl₂ (2 mM) were added to the solution and the components were further incubated with or without commercial porcine heart α-ketoglutarate dehydrogenase (1.0 mg/mL; Sigma, St. Louis, Mo.; catalog #K1502).

In a second assay, D-homoserine (2 mM; Acros, Geel, Belgium; catalog #348362500) was incubated with D-amino acid oxidase (1 U/mL; Sigma, St. Louis, Mo.; catalog #A5222) and bovine liver catalase (600 U/mL; Sigma, St. Louis, Mo.; catalog #C40) in the presence of HEPES buffer (50 mM, pH 7.3). After incubation at room temperature for 2-4 h, coenzyme A (2 mM), β-NAD⁺ (2 mM), thiamine pyrophosphate (0.2 mM), and MgCl₂ (2 mM) were added to the solution and the components were further incubated with or without purified 2-keto acid decarboxylase KdcA (1.8 μm) and propionaldehyde dehydrogenase PduP (1.8 μm).

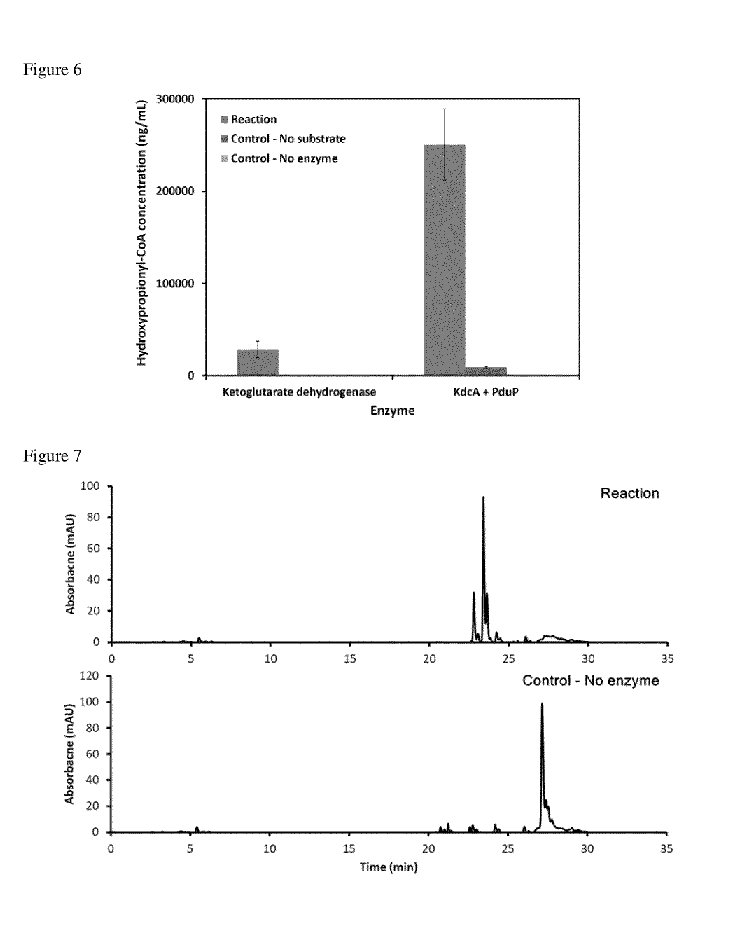

The samples were incubated at room temperature overnight, followed by LC-MS analysis to determine concentrations of 3-hydroxypropionyl-CoA as described in example 12. Only when the dehydrogenases (and decarboxylase) were present, the product was detected in significant amounts (FIG. 6).

### Example 14

### In Vitro Production of 3-hydroxypropionyl-CoA from acryloyl-CoA with 3-hydroxypropionyl-CoA dehydratase

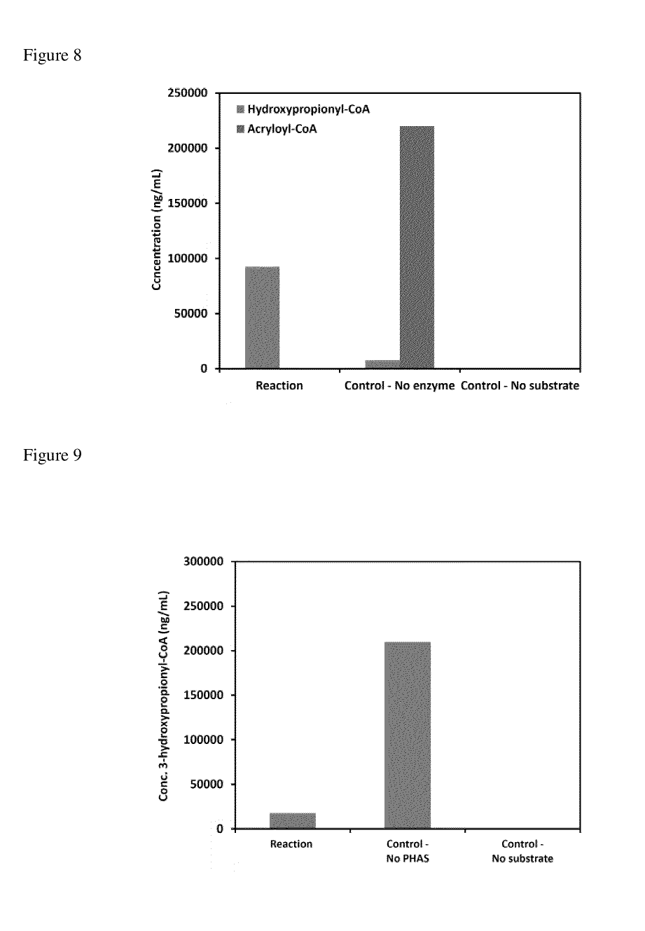

Acryloyl-CoA (1 mM) was incubated with or without 3-hydroxypropionyl-CoA dehydratase (20 μM) in the presence of HEPES buffer (50 mM, pH 7.3). After incubation at room temperature for 2-4 h, aliquots were analyzed by high performance liquid chromatography (HPLC) using an Agilent 1100 system (Agilent, Santa Clara, Calif.) monitoring absorbance at 254 nm and a Waters Atlantis T3 column (Waters, Milford, Mass.; catalog #186003748). Mobile phases were 0.1% phosphoric acid in water (A) and 0.1% phosphoric acid in 80% acetonitrile/20% water (B). Analytes were eluted isocratically at 2% B in A over 12 min, followed by a linear gradient from 2% to 35% B in A over 18 min. The HPLC analysis indicates consumption of acryloyl-CoA and formation of a different absorbing molecule (FIG. 7). The identity of the reaction product, 3-hydroxypropionyl-CoA, was confirmed by LC-MS analysis as described in example 12 (FIG. 8).

### Example 15

### In Vitro Reconstitution of PHA Synthase

A solution of 3-hydroxypropionic acid (5 mM; Aldrich, St. Louis, Mo.; catalog #AMS000335), coenzyme A (2 mM), ATP (6 mM), MgCl₂ (2 mM), and HEPES buffer (50 mM, pH 7.3) was incubated with acetyl-CoA synthetase (5 U/mL; Sigma, St. Louis, Mo.; catalog #A1765) and with or without purified PHA synthase (1 μM). After incubation at room temperature for 2-4 h, aliquots were analyzed by LC-MS as described in example 12 to determine concentrations of 3-hydroxypropionyl-CoA. When PHA synthase was present, the concentration of 3-hydroxypropionyl-CoA considerably decreased compared with a sample with no enzyme (FIG. 9).

### Example 16

### Thioesterase Activity Assay

### Ellman's Reagent

To measure relative thioesterase enzyme activity, Ellman's reagent, also known as DTNB (5,5′-dithiobis-(2-nitrobenzoic acid)), was used. The assay buffer was 50 mM KCl, 10 mM HEPES (pH 7.4). A 10 mM Ellman's reagent stock solution was prepared in ethanol. An acryloyl-CoA substrate stock solution was prepared to 10 mM in assay buffer.

For each enzyme and substrate tested, the reaction was as follows: a 10 mM Ellman's reagent stock solution was diluted to a 50 μM final concentration in assay buffer. Acryloyl-CoA stock solution was added to provide a 90 μM final concentration. The Ellman's reagent/acryloyl-CoA mixture (95 μL per well) was added to a 96-well polystyrene untreated microtiter plate. Equivalent reactions with no substrate were prepared as controls. Purified enzyme was serially diluted 1:3 in assay buffer in a separate plate, and 5 μL was added to a reaction well. Thioesterase activity was assessed at 60 min by measuring the optical density (OD) at 412 nm on a plate reader. Relative enzyme activities were calculated by subtracting OD (412 nm) of substrate-free controls from OD (412 nm) of substrate-containing samples.

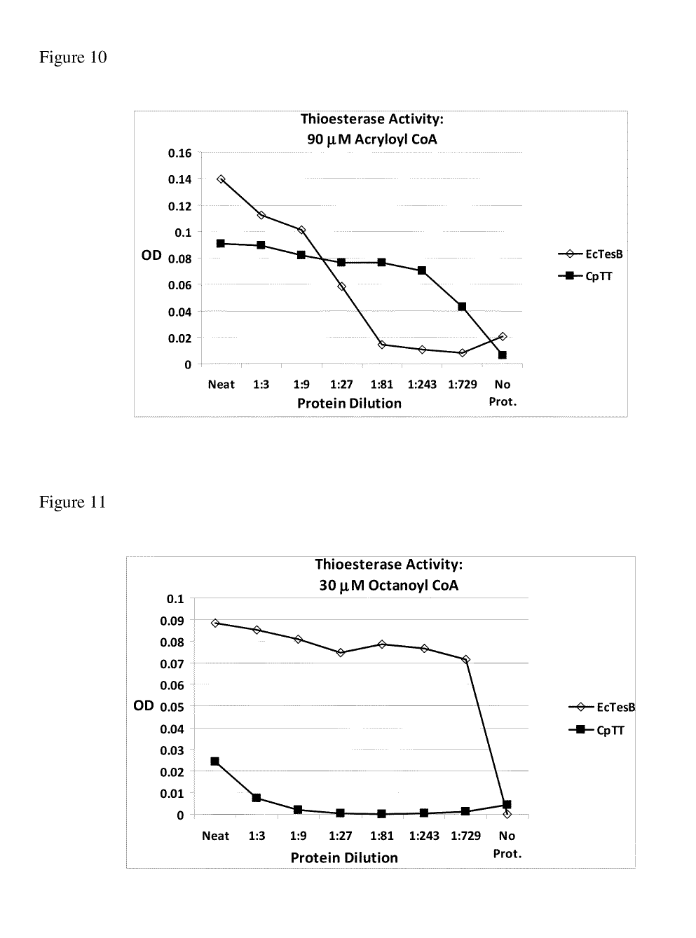

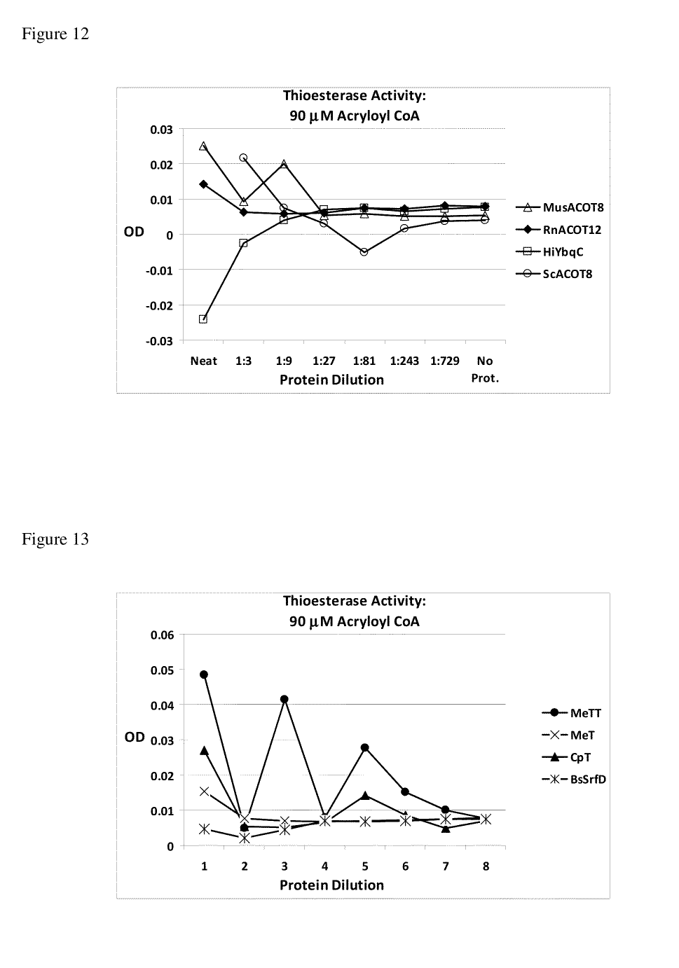

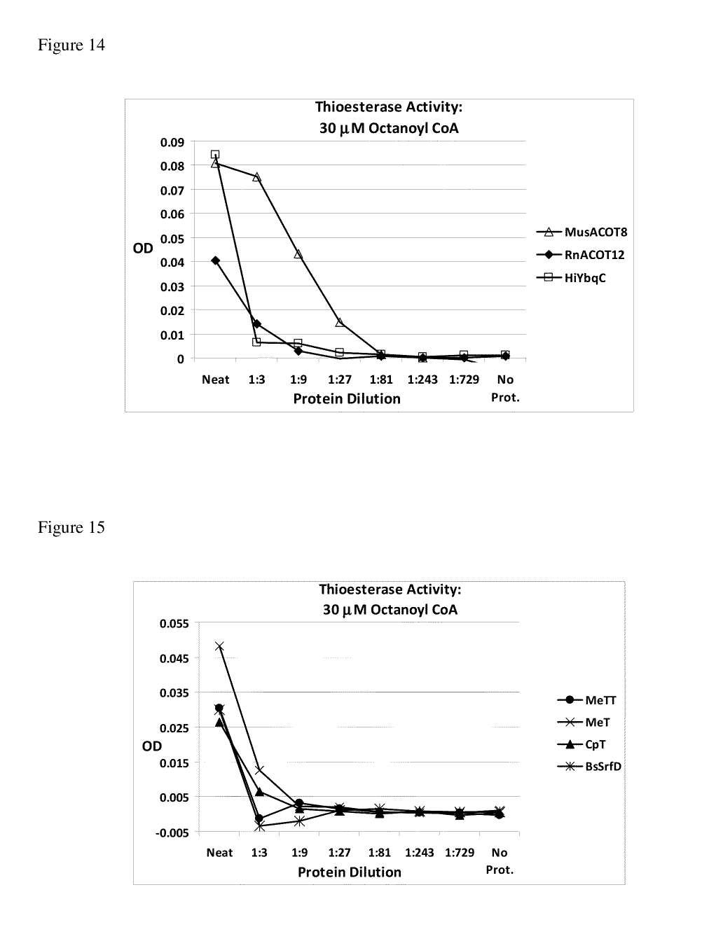

Two thioesterases, EcTesB and CpTT, each showed hydrolysis activity against the acryloyl-CoA substrate, with the activity increasing with increasing amounts of thioesterase (FIG. 10). EcTesB was also active against other substrates (FIG. 11). EcTesB hydrolyzed octanoyl-CoA, even at relatively low amounts of EcTesB. In contrast, CpTT only showed an increase in octanyol-CoA hydrolysis with the highest amounts of thioesterase (FIG. 11). The other thioesterases showed little or no thioesterase activity against acryloyl-CoA (FIGS. 10 and 11), yet their apparent hydrolysis of octanoyl-CoA suggested that the recombinant enyzmes were active (FIGS. 12 and 13). To confirm that the thioesterases were active on the coenzyme A substrate tested, samples were analyzed using liquid chromatography coupled to mass spectrometry (LC-MS) as described in example 12.

### Monitoring of Substrate and Product by LC-MS

EcTesB and CpTT showed acryloyl-CoA thioesterase activity in the assay based on generation of a free sulfhydryl from the acryloyl-CoA. As a further test of this thioesterase activity, it is useful to observe the disappearance of substrate and appearance of product. Therefore, LC-MS was used to monitor substrate and product amounts in assays with these enzymes as described in Example 12. The amount of EcTesB correlates with the increase in acryloyl-CoA hydrolysis, as indicated by both the detection of Coenzyme A by Ellman's reagent and by the disappearance of acryloyl-CoA (Table 1). As the enzyme is diluted, the thioesterase activity levels decline, as indicated by each assay. These results support EcTesB's role as a thioesterase that is active on acryloyl-CoA.

TABLE 5
Relative enzyme activity and acryloyl-CoA quantitation of TesB
thioesterase samples. The activity (OD at 412 nm) refers to the
assay based on color change in the presence of Ellman's reagent.
The acryloyl-CoA measurements were based on LC/MS.

| TesB | Activity | Acryloyl CoA |
|---|---|---|
| Dilution | OD (412 nm) | (ng)** |
| Neat | 0.140 | <200 |
| 1:3 | 0.113 | 508 |
| 1:9 | 0.101 | 14600 |
| 1:27 | 0.058 | 39400 |

**Values above 5000 are extrapolated estimates.

EcTesB and CpTT each show acryloyl-CoA hydrolysis activity by two different assays (Table 6). Each enzyme causes in increase in coenzyme A, as detected with Ellman's reagent. Each enzyme also causes a changing profile in the LC-MS analysis, with the thioesterases causing a decrease in acryloyl-CoA and an increase in coenzyme A (Table 6).

TABLE 6
Coenzyme A and acryloyl-CoA quantitation of thioesterase samples.
The activity (OD at 412 nm) refers to the assay based on color change
in the presence of Ellman's reagent. The acryloyl-CoA and coenzyme
A measurements were based on LC-MS analysis.

| Sample | Activity | | Acryloyl CoA |
|---|---|---|---|
| Name | OD (412 nm) | CoA (ng) | (ng)** |
| EcTesB | 0.2157 | 28100 | 756 |
| CpTT | 0.0992 | 13600 | 1430 |
| no enzyme | 0.051 | 259 | 79000 |

### Example 17

### Production of 3-hydroxypropionic acid in Engineered E. coli

This example demonstrates increased production of 3-hydroxypropionic acid in E. coli host cells which can then be converted to poly-3-hydroxypropionic acid or acylic acid. E. coli strains were established to overexpress P. fluorescens branched-chain amino acid aminotransferase (Pf AT) set out in SEQ ID NO: 8, L. lactis branched-chain 2-keto acid decarboxylase (KdcA) set out in SEQ ID NO: 54, S. enterica coenzyme-A acylating propionaldehyde dehydrogenase (PduP) set out in SEQ ID NO: 60, and in some instances C. necator Poly(3-hydroxybutyrate) polymerase (PhaC) set out in SEQ ID NO: 42.

In this example, P. fluorescens branched-chain amino acid aminotransferase (SEQ ID NO: 8) promoted the conversion of L-homoserine to 2-keto-4-hydroxybutyrate. The L. lactis branched-chain 2-keto acid decarboxylase (KdcA, set out in SEQ ID NO: 540 catalyzed the conversion of 2-keto-4-hydroxybutyrate to 3-hydroxy-propionaldehyde. The S. enterica coenzyme-A acylating propionaldehyde dehydrogenase (PduP, set out in SEQ ID NO: 60) catalyzed the conversion of 3-hydroxy-propionaldehyde to 3-hydroxypropionyl-CoA. A thioesterase catalyzed the conversion of 3-hydroxypropionyl-CoA to 3-hydroxypropionate. Alternative, the C. necator Poly (3-hydroxybutyrate) polymerase (PhaC, set out in SEQ ID NO: 42) can catalyze the conversion of 3-hydroxypropionyl-CoA to poly-3-hydroxypropionate.

### Plasmid Construction

An E. coli expression vector was constructed for overexpression of a recombinant P. fluorescens branched-chain amino acid aminotransferase (Pf AT) and C. necator Poly (3-hydroxybutyrate) polymerase (PhaC). The codon optimized C. necator Poly (3-hydroxybutyrate) polymerase (phaC) from pET30a-BB Cn PHAS (Example 4) was cloned into pET30a-BB Pf AT (Example 1) by double digestion of pET30a-BB Cn PHAS with restriction enzymes XbaI and PstI. The Cn PHAS fragment was band isolated, purified using a QIAquick Gel Extraction Kit (Qiagen, Carlsbad, Calif.) and ligated (Fast-Link Epicentre Biotechnologies, Madison, Wis.) with SpeI/PstI-digested pET30a-BB Pf AT vector. The ligation mix was used to transform OneShot Top10™ E. coli cells (Invitrogen, Carlsbad, Calif.).

Individual vials of cells were thawed on ice and gently mixed with 2 μL of ligation mix. The vials were incubated on ice for 30 min. The vials were briefly incubated at 42° C. for 30 sec and quickly replaced back on ice for an additional 2 min. 250 μL of 37° C. SOC medium was added and the vials were secured horizontally on a shaking incubator platform and incubated for 1 h at 37° C. and 225 rpm. Aliquots of 20 μL and 200 μL cells were plated onto LB agar supplemented with kanamycin (50 μg/mL). Single colony isolates were isolated and cultured in 5 mL of LB broth with kanamycin (50 μg/mL). The recombinant plasmid was isolated using a Qiagen Plasmid Mini Kit and characterized by gel electrophoresis of restriction digests with MITI. The resulting plasmid was designated pET30a-BB Pf AT_Cn PHAS.

An E. coli expression vector was constructed for overexpression of a recombinant S. enterica coenzyme-A acylating propionaldehyde dehydrogenase (PduP) and L. lactis branched-chain 2-keto acid decarboxylase (KdcA). The codon optimized L. lactis branched-chain 2-keto acid decarboxylase (kdcA) from pET30a-BB Ll KDCA (Example 2) was cloned into pET30a-BB Se PDUP (Example 3) by double digestion of pET30a-BB Ll KDCA with restriction enzymes XbaI and

PstI. The Ll KDCA fragment was band isolated, purified using a QIAquick Gel Extraction Kit (Qiagen, Carlsbad, Calif.) and ligated (Fast-Link Epicentre Biotechnologies, Madison, Wis.) with SpeI/PstI-digested pET30a-BB Se PDUP vector. The ligation mix was used to transform OneShot Top10™ E. coli cells (Invitrogen, Carlsbad, Calif.). Individual vials of cells were thawed on ice and gently mixed with 2 μL of ligation mix. The vials were incubated on ice for 30 min. The vials were briefly incubated at 42° C. for 30 sec and quickly replaced back on ice for an additional 2 min. 250 μL of 37° C. SOC medium was added and the vials were secured horizontally on a shaking incubator platform and incubated for 1 h at 37° C. and 225 rpm. Aliquots of 20 μL and 200 μL cells were plated onto LB agar supplemented with kanamycin (50 μg/mL). Single colony isolates were isolated and cultured in 5 mL of LB broth with kanamycin (50 μg/mL) and the recombinant plasmid was isolated using a Qiagen Plasmid Mini Kit and characterized by gel electrophoresis of restriction digests with AflIII. The resulting plasmid was designated pET30a-BB Se PDUP_Ll KDCA.

To facilitate cotransformation with pET30a-BB Pf AT or pET30a-BB Pf AT_Cn PHAS, the codon optimized S. enterica coenzyme-A acylating propionaldehyde dehydrogenase (pduP) and L. lactis Branched-chain 2-keto acid decarboxylase (kdcA) gene pair was subcloned from pET30a-BB Se PDUP_Ll KDCA into the pCDFDuet-1 vector (Novagen, EMD Chemicals, Gibbstown, N.J.; catalog #71340-3) by double digestion of pET30a-BB Se PDUP_Ll KDCA with restriction enzymes EcoRI and PstI. The Se PDUP_Ll KDCA fragment was band isolated, purified using a QIAquick Gel Extraction Kit (Qiagen, Carlsbad, Calif.) and ligated (Fast-Link Epicentre Biotechnologies, Madison, Wis.) with EcoRI/PstI-digested pCDFDuet-1. The ligation mix was used to transform OneShot Top10™ E. coli cells (Invitrogen, Carlsbad, Calif.). Individual vials of cells were thawed on ice and gently mixed with 2 μL of ligation mix. The vials were incubated on ice for 30 min. The vials were briefly incubated at 42° C. for 30 sec and quickly replaced back on ice for an additional 2 min. 250 μL of 37° C. SOC medium was added and the vials were secured horizontally on a shaking incubator platform and incubated for 1 h at 37° C. and 225 rpm. Aliquots of 20 μL and 200 μL cells were plated onto LB agar supplemented with spectinomycin (50 μg/mL). Single colony isolates were isolated, cultured in 5 mL of LB broth with spectinomycin (50 μg/mL) and the recombinant plasmid was isolated using a Qiagen Plasmid Mini kit and characterized by gel electrophoresis of restriction digests with AflIII. The resulting plasmid was designated pCDFDuet-1 Se PDUP_Ll KDCA.

Co-Transformation of E. coli

The recombinant plasmids and empty parent vectors were used to co-transform chemically competent BL21 (DE3) pLysS E. coli cells (Invitrogen, Carlsbad, Calif.) in the following combinations:

pET30a-BB Pf AT_Cn PHAS and pCDFDuet-1 Se PDUP_Ll KDCA

pET30a-BB Pf AT and pCDFDuet-1 Se PDUP_Ll KDCA

pET30a-BB and pCDFDuet-1

Individual vials of cells were thawed on ice and gently mixed with 50 ng of plasmid DNA. The vials were incubated on ice for 30 min. The vials were briefly incubated at 42° C. for 30 sec and quickly replaced back on ice for an additional 2 min. 250 μL of 37° C. SOC medium was added and the vials were secured horizontally on a shaking incubator platform and incubated for 1 h at 37° C. and 225 rpm. Aliquots of 20 μL and 200 μL cells were plated onto LB agar supplemented with the appropriate antibiotics (50 μg/mL kanamycin; 50 μg/mL spectinomycin; 34 μg/mL chloramphenicol) to select for cells carrying the recombinant pET30a-BB, pCDFDuet-1 and pLysS plasmids respectively and incubated overnight at 37° C. Single colony isolates were isolated, cultured in 5 mL of selective LB broth and the recombinant plasmids were isolated using a QIAPrep® Spin Miniprep Kit (Qiagen, Valencia, Calif.) and characterized by gel electrophoresis of restriction digests with AflIII.

### Strain Culture

Single colony forming units of E. coli BL21 (DE3) pLysS cells co-transformed with the described plasmids were used to inoculated aliquots of minimal M9 broth (25 mL) supplemented with the appropriate antibiotics (34 μg/mL chloramphenicol, 50 μg/mL kanamycin, and 50 μg/mL spectinomycin). The cultures were incubated overnight at 37° C. with shaking at 250 rpm and used to inoculated fresh minimal M9 media (50 mL) supplemented with the same antibiotics. After overnight incubation under similar conditions, aliquots of cultures were used to inoculate a new set of M9 broths (50 mL) with antibiotics (34 μg/mL chloramphenicol, 50 μg/mL kanamycin, and 50 μg/mL spectinomycin) and supplemented with or without L-homoserine (1 g/L; Sigma, St. Louis, Mo.), followed by incubation at 25° C. with shaking at 250 rpm. When OD₆₀₀ of about 0.2 was reached, protein expression was induced by addition of 50 μL of 1M IPTG (1 mM final concentration; Teknova, Hollister, Calif.), followed by incubation for 17 h at 25° C. with 250 rpm shaking. Cells were harvested by centrifugation and supernatants were filtered through Acrodisc Syringe Filters (0.2 μm HT Tuffryn membrane; low protein binding; Pall Corporation, Ann Arbor, Mich.) and frozen on dry ice prior to storage at −80° C. until analysis.

| Minimal M9 Media | | |
|---|---|---|
| Component | 1X Base Recipe | |
| Na₂HPO₄ | 6 | g/L |
| KH₂PO₄ | 3 | g/L |
| NaCl | 0.5 | g/L |
| NH₄Cl | 1 | g/L |
| CaCl₂ * 2H₂O | 0.1 | mM |
| MgSO₄ | 1 | mM |
| Dextrose | 80 | mM |
| Thiamine | 1 | mg/L |
| Chloramphenicol | 34 | μg/mL |
| Kanamycin | 50 | μg/mL |
| Spectinomycin | 50 | μg/mL |

Detection of 3-hydroxypropionic acid by Engineered E. coli

An internal standard solution of 100 μg/mL of ¹³C₃-labelled lactic acid in 1:1 MeOH:H₂O was prepared. External standard solutions were prepared at 3-hydroxypropanoic acid concentrations of 1 μg/mL, 2.5 μg/mL, 5 μg/mL, 10 μg/mL and 25 μg/mL in 1:1 MeOH:H₂O. 900 μL of filtered supernatant or external standard was added to 100 μL of the internal standard solution. These solutions were subjected to ion exclusion liquid chromatography (LC) separations and mass spectrometry (MS) detection.

The LC separation conditions were as follows: 10 μL of sample/standard were injected onto a Thermo Fisher Dionex ICE-AS1 (4×250 mm) column (with guard) running an isocratic mobile phase of 1 mM heptafluorbutyric acid at a flow rate of 0.15 mL/min. 20 mM NH₄OH in MeCN at 0.15 mL/min was teed into the column effluent.

The MS detection conditions were as follows: A Sciex API-4000 MS was run in negative ion mode and monitored the m/z 89 to 59 (unit resolution) transition of 3-hydroxypropanoic acid and the m/z 92 to 45 (unit resolution) transition of ¹³C₃-labelled lactic acid. The dwell time used was 300 ms, the declustering potential was set at −38, the entrance potential was set at −10, the collision gas was set at 12, the curtain gas was set at 15, the ion source gas 1 was set at 55, the ion source gas 2 was set at 55, the ionspray voltage was set at −3500, the temperature was set at 650, the interface heater was on. For 3-hydroxypropanoic acid, the collision energy was set at −16 and the collision set exit potential was set at −9. For ¹³C₃-labelled lactic acid, the collision energy was set at −18 and the collision set exit potential was set at −16.

The results of the analysis are shown in Table 7. The data evidenced that increased levels of 3-hydroxypropanoic acid were produced when Pf AT, KdcA, and PduP were overexpressed and L-homoserine was supplemented to the culture media. Endogenous L-homoserine and E. coli proteins likely supported production of small amounts of 3-hydroxypropionic acid when no exogenous homoserine was added to the culture medium and/or when empty pET30a-BB and pCDF Duet-1 vectors were present.

TABLE 7
Production of 3-hydroxypropionic acid by engineered E. coli

| | Addition of | Concentration of |
|---|---|---|
| | L- | 3-hydroxypropionic |
| Plasmids | homoserine? | acid (μg/mL) |
| pET30a-BB Pf_AT::Cn_PHAS and | No | 0.08 |
| pCDF Duet-1 Se_PDUP::Ll_KDCA | | |
| pET30a-BB Pf_AT and | No | 0.08 |
| pCDF Duet-1 Se_PDUP::Ll_KDCA | | |
| pET30a-BB and | No | 0.15 |
| pCDF Duet-1 | | |
| pET30a-BB Pf_AT::Cn_PHAS and | Yes | 2.00 |
| pCDF Duet-1 Se_PDUP::Ll_KDCA | | |
| pET30a-BB Pf_AT and | Yes | 4.13 |
| pCDF Duet-1 Se_PDUP::Ll_KDCA | | |
| pET30a-BB and | Yes | 0.73 |
| pCDF Duet-1 | | |

While the present invention has been described in terms of specific embodiments, it is understood that variations and modifications will occur to those skilled in the art. Accordingly, only such limitations as appear in the claims should be placed on the invention.

All documents referred to in this application are hereby incorporated by reference in their entirety.

1. A method for converting homoserine to 3-hydroxypropionyl-CoA comprising the steps of:
a) converting homoserine to 2-keto-4-hydroxybutyrate, wherein this conversion is catalyzed by at least one enzyme selected from the group consisting of an aminotransferase, an L-amino acid oxidase and an L-amino acid dehydrogenase; and
b) converting 2-keto-4-hydroxybutyrate to 3-hydroxypropionyl-CoA, wherein this conversion is catalyzed by at least one enzyme selected from the group consisting of a 2-ketoacid dehydrogenase and a combination of a 2-ketoacid decarboxylase and a dehydrogenase.

2. The method of claim 1 in which a recombinant microorganism overexpresses one or more genes to convert homoserine to 3-hydroxypropionyl-CoA.

3. The method of claim 2 in which the microorganism expresses a poly-3-hydroxyalkanoate synthase to further convert 3-hydroxypropionyl-CoA to a poly-3-hydroxyalkanoate containing 3-hydroxypropionate monomers.

4. The method of claim 1 further comprising the steps of:
c) converting 3-hydroxypropionyl-CoA to acryloyl-CoA, wherein this conversion is catalyzed by a dehydratase; and
d) converting acryloyl-CoA to acrylic acid, wherein this conversion is catalyzed by at least one enzyme selected from the group consisting of a thioesterase, a CoA-transferase, and a combination of a phosphate transferase and kinase.

5. The method of claim 4 in which a recombinant microorganism converts homoserine to acrylic acid.

6. The method of claim 1 in which 3-hydroxypropionyl-CoA is further converted to 3-hydroxypropionic acid by a microorganism expressing an enzyme selected from the group consisting of a transferase and a thioesterase.

7. (canceled)

8. (canceled)

9. (canceled)

10. (canceled)

11. (canceled)

12. (canceled)

13. (canceled)

14. (canceled)

15. (canceled)

16. (canceled)

17. The microorganisms of claim 1 in which the threonine pathway has been engineered to increase carbon flux to homoserine when compared to a wild type microorganism.

18. The microorganisms of claim 1 in which the oxaloacetate synthesis has been engineered to increase carbon flux to homoserine when compared to a wild type microorganism.

19. (canceled)

20. (canceled)


SEQUENCE LISTING (127 sequences)

SEQ ID NO: 1 - DNA, 2001 bp, Sus scrofa
gagcggctccgggcgcgaggtgaaagctcccggccgactcctgctctctagctatggcacctccatcagtctttgccgaggttcctcaggcccagccggtccttgtctttaagctcattgctgacttccgggaggatccggacccccgcaaggtcaacctaggagtgggagcttatcgcaccgatgattgccagccttgggttttgccagtcgtgaggaaggtggagcagaggattgctaatgacagcagcctaaaccacgagtacctgcccatcctgggcctggcagagttccggacctgtgcttcccgccttgcccttggagatgacagcccagctcttcaggagaagcgggtgggaggggtgcagtctttggggggaacgggtgcacttcgaattggagctgagttcttagcacgatggtacaatggaacgaacaacaaagacacgcctgtctacgtatcctcaccaacctgggaaaatcacaatggagtcttcactactgctggattcaaagacattcggtcctatcgctattgggataccgagaagagaggacttgatctccagggtttcctgagtgatctggagaacgctcctgagttctccatctttgtcctccacgcctgtgcccacaacccgacagggaccgacccaactccggagcaatggaagcagatcgcctctgtcatgaagcgccggtttctgttccccttctttgactcagcctatcagggcttcgcatctggcaacctagaaaaagacgcctgggccattcgctattttgtgtctgaagggttcgagctcttctgtgcccagtccttctccaagaacttcgggctctacaatgagcgcgtggggaacctgaccgtggttgcaaaagaacccgatagcatcctgcgagtcctttcccagatggagaagatcgtgcgagtgacgtggtccaatccccctgctcagggagcgagaatcgtggcccgtacgctctctgaccctgagctctttcatgaatggacaggtaacgtgaagacaatggctgaccgcattctgagcatgagatctgagcttagggcacgattagaagccctcaagacccctggaacctggaaccacatcacggaccagattggaatgttcagcttcactgggttgaaccccaagcaggttgaatatctgatcaacgaaaagcacatctatctgctaccaagtggtcggatcaacatgtgtggcttaaccaccaaaaatctagattatgtggccacctccatccatgaagctgtcaccaaaatccagtgaagcaacaccacccaagccagcgccacccaagcggtcctctgtctcgtgtgttccctgcctgcacaaacctggttctatacatcacaactgtattagaggctaccgagggacagaaaaggctgctctggtgaggtagctgctatttaaattggccccatgggaagagaacatctcttgaaaagaaatgggggccagggaatagagcccttttggaggccagagcaaattcaggcttttatttgaaaagaataaaaaggtcctttgatcatgagatgtagatgtcttgccccctcactagaagcaggagtattgcctgtgtcactcacgtgctcctgtgtgttttactctgtacaaagtctagtcccaaagatcaagttgtctgaagagcaaagtgtgattgtgggtattggctgtgtcattaacagttgtcctctggacccagagtgtctgtctccctgctctttctgcatggctctgtccctagccctaagcttgagttctttagggtggtcaaggtaggaaatatatttatattttacccacacgttaactgaaataaaagtttcacagagtcaaatttacccttactatgtggagtacattctggtattttcttttctattctattctattctattctattctattctattctattctattctgttgtttcactaaagaaataaaagtgctgattgagacccat

SEQ ID NO: 2 - protein, 413 aa, Sus scrofa
MAPPSVFAEVPQAQPVLVFKLIADFREDPDPRKVNLGVGAYRTDDCQPWVLPVVRKVEQRIANDSSLNHEYLPILGLAEFRTCASRLALGDDSPALQEKRVGGVQSLGGTGALRIGAEFLARWYNGTNNKDTPVYVSSPTWENHNGVFTTAGFKDIRSYRYWDTEKRGLDLQGFLSDLENAPEFSIFVLHACAHNPTGTDPTPEQWKQIASVMKRRFLFPFFDSAYQGFASGNLEKDAWAIRYFVSEGFELFCAQSFSKNFGLYNERVGNLTVVAKEPDSILRVLSQMEKIVRVTWSNPPAQGARIVARTLSDPELFHEWTGNVKTMADRILSMRSELRARLEALKTPGTWNHITDQIGMFSFTGLNPKQVEYLINEKHIYLLPSGRINMCGLTTKNLDYVATSIHEAVTKIQ

SEQ ID NO: 3 - DNA, 1744 bp, Rattus norvegicus
ctgcagtgtgaagggtgttgtcttacctctcgcagactttcccattcccagccctgatttccccactggacccttctccttctgaaccagcatcctgcctggtttgagcagtcatggcctcacgggtgaatgatcaaagccaggcttcaaggaatgggctgaagggaaaggtgctaactctggacactatgaacccatgtgtgcggagggtggagtatgcagttcgaggacccattgtgcagcgtgccttggagctggagcaggagctgcgtcagggtgtgaagaagccgtttactgaggtcatccgtgccaacattggggatgcacaagccatggggcagagacccatcaccttcttccgccaggtcctggccctctgtgtctaccccaatcttctgagcagtcctgacttcccagaggatgccaagagaagggcagaacgcatcttgcaggcctgcgggggccacagcctgggtgcctatagcattagctctggaatccagccgatccgggaggatgtggcgcaatacattgagagaagagacggaggcatccccgcagacccgaacaacatatttctatccacaggggccagcgatgccatcgtgacaatgctcaagctgctggtatctggcgagggccgtgcacgaacaggtgtactcattcccattcctcagtacccactgtactcagccgcgctggctgaactggacgccgtgcaagtggactactacctggacgaagagcgcgcctgggctctggacatcgcagagctgcggcgcgctctgtgccaggcacgtgaccgttgctgccctcgagtactgtgcgtcatcaaccccggcaaccccactgggcaggtgcagacccgtgagtgcatcgaggccgtaatccgctttgctttcaaagaaggactcttcttgatggctgatgaggtataccaggacaacgtgtatgccgagggctctcagttccattcattcaagaaggtgctcatggagatggggccaccgtattccacgcagcaggagcttgcttctttccactcagtctctaagggctacatgggcgagtgcgggtttcgtggtggctatgtggaggtggtaaacatggatgctgaggtgcagaaacagatggggaagctgatgagtgtgcggctgtgtccaccagtgccaggccaggccttgatggacatggtggtcagtccgccaacaccctccgagccgtccttcaagcagtttcaagcagagagacaggaggtgctggctgaactggcagccaaggctaagctcacggagcaggtcttcaatgaggctcccgggatccgctgcaacccagtgcagggcgccatgtattccttccctcaagtgcagctgcccttgaaagcggtgcagcgtgctcaggaactgggcctggcccctgacatgttcttctgcctgtgcctcctggaagagactggcatctgcgttgtgcccgggagtggctttgggcagcaggagggcacctatcatttccggatgaccattctgccccccatggagaaactgcggctgctgctggaaaaactcagtcacttccatgccaagttcacccatgagtactcctgaagccactgctagggccacactggacagtctctgacgcaacaaaccgagggtccttaggaaccctcagtatttctgattttgtctagggtctcggtaactgtcctgcgggtccctaataaatctgatgtcagcctgaaaaa

SEQ ID NO: 4 - protein, 496 aa, Rattus norvegicus
MASRVNDQSQASRNGLKGKVLTLDTMNPCVRRVEYAVRGPIVQRALELEQELRQGVKKPFTEVIRANIGDAQAMGQRPITFFRQVLALCVYPNLLSSPDFPEDAKRRAERILQACGGHSLGAYSISSGIQPIREDVAQYIERRDGGIPADPNNIFLSTGASDAIVTMLKLLVSGEGRARTGVLIPIPQYPLYSAALAELDAVQVDYYLDEERAWALDIAELRRALCQARDRCCPRVLCVINPGNPTGQVQTRECIEAVIRFAFKEGLFLMADEVYQDNVYAEGSQFHSFKKVLMEMGPPYSTQQELASFHSVSKGYMGECGFRGGYVEVVNMDAEVQKQMGKLMSVRLCPPVPGQALMDMVVSPPTPSEPSFKQFQAERQEVLAELAAKAKLTEQVFNEAPGIRCNPVQGAMYSFPQVQLPLKAVQRAQELGLAPDMFFCLCLLEETGICVVPGSGFGQQEGTYHFRMTILPPMEKLRLLLEKLSHFHAKFTHEYS

SEQ ID NO: 5 - DNA, 1182 bp, Saccharomyces cerevisiae
atgttgcagagacattccttgaagttggggaaattctccatcagaacactcgctactggtgccccattagatgcatccaaactaaaaattactagaaacccaaatccatccaagccaagaccaaatgaagaattagtgttcggccagacattcaccgatcatatgttgaccattccttggtcagccaaagaagggtggggcactccacacatcaagccttacggtaatctttctcttgacccatctgcttgtgtattccattatgcatttgaattatttgaaggtttgaaagcctacagaactcctcaaaatactatcaccatgttccgtccggataagaacatggcccgtatgaacaagtctgccgctagaatttgtttgccaactttcgaatctgaagaattgatcaaacttaccgggaaattgatcgaacaagataaacacttggttcctcaaggtaatggttactcattatacatcagaccaacaatgattggtacatccaagggtttaggtgttggcactccctccgaggctcttctttatgttattacttctccagtcggtccttattataagactggtttcaaagccgtacgtcttgaagcaacagactatgctacaagagcttggccaggtggtgttggcgacaaaaaattgggtgctaactatgccccatgcatcttacctcaactacaagctgccaaaagagggtaccaacaaaatctatggttgttcggcccagaaaagaacatcactgaggttggtactatgaacgtgttcttcgttttcctcaacaaagtcactggcaagaaggaattggttaccgctccattagatggtaccattttagaaggtgttaccagagactctgttttaacattggctcgtgacaaactagatcctcaagaatgggacatcaacgagcgttattacactattactgaagtcgccactagagcaaaacaaggtgaactattagaagccttcggttctggtactgctgctgtcgtttcacctatcaaggaaattggctggaacaacgaagatattcatgttccactattgcctggtgaacaatgtggtgcattgaccaagcaagttgctcaatggattgctgatatccaatacggtagagtcaattatggtaactggtcaaaaactgttgccgacttgaactaa

SEQ ID NO: 6 - protein, 393 aa, Saccharomyces cerevisiae
MLQRHSLKLGKFSIRTLATGAPLDASKLKITRNPNPSKPRPNEELVFGQTFTDHMLTIPWSAKEGWGTPHIKPYGNLSLDPSACVFHYAFELFEGLKAYRTPQNTITMFRPDKNMARMNKSAARICLPTFESEELIKLTGKLIEQDKHLVPQGNGYSLYIRPTMIGTSKGLGVGTPSEALLYVITSPVGPYYKTGFKAVRLEATDYATRAWPGGVGDKKLGANYAPCILPQLQAAKRGYQQNLWLFGPEKNITEVGTMNVFFVFLNKVTGKKELVTAPLDGTILEGVTRDSVLTLARDKLDPQEWDINERYYTITEVATRAKQGELLEAFGSGTAAVVSPIKEIGWNNEDIHVPLLPGEQCGALTKQVAQWIADIQYGRVNYGNWSKTVADLN

SEQ ID NO: 7 - DNA, 1020 bp, Pseudomonas fluorescens
atgggtaacgaaagcatcaattgggacaagctgggttttgactacatcaagaccgacaagcggtttctccaggtctggaaaaacggcgaatggcaagctggcaccctgaccgacgacaacgtgctgcacatcagcgagggctccaccgccctgcactatggccagcaatgctttgaaggcctcaaggcctaccgctgcaaggacggctcgatcaacctgttccgcccggaccagaacgccgcccgcatgcagcgcagctgcgcgcgcctgctgatgccgcatgtgtcgaccgaagacttcatcgacgcctgcaaacaagtggtcaaggccaacgagcgcttcatcccgccgtatggcagtggcggcgcgctgtacctgcgcccgttcgtgatcggcaccggtgacaacatcggtgtgcgtaccgcgccggagttcatcttctcggtgttcgccatcccggttggcgcctacttcaaaggcggcctggtaccacacaacttccagatctccaccttcgaccgcgccgcaccccagggcaccggtgccgccaaggtcggtggcaactacgccgccagcctgatgccgggcgccgaagcgaagaaatccggtttcgccgatgcgatctacctggacccgatgactcactcgaaaatcgaagaagtcggctcggccaacttcttcgggatcacccacgacaataagttcatcacaccgaagtcgccttcggtgctgccaggcatcacccgtctgtcgctgatcgaactggccaagacccgcctgggcctggaagtggtcgagggcgaagtgttcatcgacaaactggaccagttcaaggaagccggtgcctgcggtaccgccgcggtgatctcgccgatcggcggtatccagtacaacggcaagctgcacgtgttccacagcgagaccgaagtcggcccgatcacccagaagctctacaaagagctgaccggcgtgcagaccggcgacgttgaagcgccagcgggctggatcgtcaaggtctaa

SEQ ID NO: 8 - protein, 339 aa, Pseudomonas fluorescens
MGNESINWDKLGFDYIKTDKRFLQVWKNGEWQAGTLTDDNVLHISEGSTALHYGQQCFEGLKAYRCKDGSINLFRPDQNAARMQRSCARLLMPHVSTEDFIDACKQVVKANERFIPPYGSGGALYLRPFVIGTGDNIGVRTAPEFIFSVFAIPVGAYFKGGLVPHNFQISTFDRAAPQGTGAAKVGGNYAASLMPGAEAKKSGFADAIYLDPMTHSKIEEVGSANFFGITHDNKFITPKSPSVLPGITRLSLIELAKTRLGLEVVEGEVFIDKLDQFKEAGACGTAAVISPIGGIQYNGKLHVFHSETEVGPITQKLYKELTGVQTGDVEAPAGWIVKV

SEQ ID NO: 9 - DNA, 1218 bp, Escherichia coli
atgtcccccattgaaaaatccagcaaattagagaatgtctgttatgacatccgtggtccggtgctgaaagaagcaaaacgcctggaagaagaaggtaacaaggtactgaaactgaacatcggcaacccagccccgttcggttttgacgcgccagatgaaatcctcgttgacgtgatacgcaacctgcctaccgctcaagggtattgcgattccaaaggtctttactccgcgcgtaaagccatcatgcagcactaccaggctcgtggcatgcgtgatgttaccgtggaagatatttacatcggcaatggtgtatcggagcttatcgttcaggcaatgcaggcattgctgaacagcggggacgaaatgttggttcctgcaccagattacccactctggaccgcggcggtttcgctttccagcggtaaagcggtgcattatctttgcgatgaatcctctgactggttcccggacctcgatgatattcgcgctaaaattacgcctcgtacgcgtgggatcgttattatcaacccaaataacccaaccggcgcggtatattccaaagagcttttaatggagattgtggagattgcacgtcagcataatctcattatcttcgccgatgaaatttatgacaaaattctctacgacgacgctgagcatcactcaattgcgccgctggcacctgacctgctgaccattacctttaacggactgtcgaaaacgtaccgcgttgcaggcttccgtcaggggtggatggtgttgaacgggccgaaaaaacacgccaaaggctacatcgaaggtctggaaatgctggcttcaatgcgcctgtgtgctaacgttcctgcgcaacacgccattcagaccgcgctaggtggttatcagagcatcagtgaatttattacccctggcggtcgtctttatgagcagcgtaaccgcgcgtgggaactgatcaacgatattccgggcgtttcctgcgtgaaacctcgtggtgcgctgtatatgttcccgaaaatcgacgccaaacgctttaacattcacgacgatcagaaaatggtgttggatttcctgttgcaggaaaaagttctgttggtgcaagggacggcattcaactggccgtggccggatcacttccgcattgtcacgctaccgcgtgtcgatgatatcgagctgtctttgagcaagttcgcgcgtttcctttctggttatcatcagctgtaa

SEQ ID NO: 10 - protein, 405 aa, Escherichia coli
MSPIEKSSKLENVCYDIRGPVLKEAKRLEEEGNKVLKLNIGNPAPFGFDAPDEILVDVIRNLPTAQGYCDSKGLYSARKAIMQHYQARGMRDVTVEDIYIGNGVSELIVQAMQALLNSGDEMLVPAPDYPLWTAAVSLSSGKAVHYLCDESSDWFPDLDDIRAKITPRTRGIVIINPNNPTGAVYSKELLMEIVEIARQHNLIIFADEIYDKILYDDAEHHSIAPLAPDLLTITFNGLSKTYRVAGFRQGWMVLNGPKKHAKGYIEGLEMLASMRLCANVPAQHAIQTALGGYQSISEFITPGGRLYEQRNRAWELINDIPGVSCVKPRGALYMFPKIDAKRFNIHDDQKMVLDFLLQEKVLLVQGTAFNWPWPDHFRIVTLPRVDDIELSLSKFARFLSGYHQL

SEQ ID NO: 11 - DNA, 1191 bp, Escherichia coli
atgtttgagaacattaccgccgctcctgccgacccgattctgggcctggccgatctgtttcgtgccgatgaacgtcccggcaaaattaacctcgggattggtgtctataaagatgagacgggcaaaaccccggtactgaccagcgtgaaaaaggctgaacagtatctgctcgaaaatgaaaccaccaaaaattacctcggcattgacggcatccctgaatttggtcgctgcactcaggaactgctgtttggtaaaggtagcgccctgatcaatgacaaacgtgctcgcacggcacagactccggggggcactggcgcactacgcgtggctgccgatttcctggcaaaaaataccagcgttaagcgtgtgtgggtgagcaacccaagctggccgaaccataagagcgtctttaactctgcaggtctggaagttcgtgaatacgcttattatgatgcggaaaatcacactcttgacttcgatgcactgattaacagcctgaatgaagctcaggctggcgacgtagtgctgttccatggctgctgccataacccaaccggtatcgaccctacgctggaacaatggcaaacactggcacaactctccgttgagaaaggctggttaccgctgtttgacttcgcttaccagggttttgcccgtggtctggaagaagatgctgaaggactgcgcgctttcgcggctatgcataaagagctgattgttgccagttcctactctaaaaactttggcctgtacaacgagcgtgttggcgcttgtactctggttgctgccgacagtgaaaccgttgatcgcgcattcagccaaatgaaagcggcgattcgcgctaactactctaacccaccagcacacggcgcttctgttgttgccaccatcctgagcaacgatgcgttacgtgcgatttgggaacaagagctgactgatatgcgccagcgtattcagcgtatgcgtcagttgttcgtcaatacgctgcaggaaaaaggcgcaaaccgcgacttcagctttatcatcaaacagaacggcatgttctccttcagtggcctgacaaaagaacaagtgctgcgtctgcgcgaagagtttggcgtatatgcggttgcttctggtcgcgtaaatgtggccgggatgacaccagataacatggctccgctgtgcgaagcgattgtggcagtgctgtaa

SEQ ID NO: 12 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 13 - DNA, 2533 bp, Calloselasma rhodostoma
ggcacgagctttgcttagcatcagtaacttttcttccaagcattgccatccacagacttcaaaccaataagatgaatgtcttctttatgttctcgctgctgttcttggctgccttgggaagctgtgcagatgacagaaaccctctagcggaatgcttccaagaaaatgactatgaagaatttctagagatcgccagaaatggtctgaaagcgacatcaaacccaaaacatgttgtgattgtaggtgcaggaatggctgggcttagtgcagcctatgttcttgcaggggctggacatcaggtgacagttcttgaagccagtgaacgtccgggaggacgagtgagaacttatcgaaatgaggaagcaggctggtatgccaatctcgggcccatgcgtttacctgagaaacacaggattgtccgggaatatatcagaaagtttgatctccggttgaatgaattttctcaggaaaatgacaatgcctggtattttatcaaaaacatcaggaagaaagttggggaagtcaagaaagaccctggcctcttgaaatatcccgtgaagccttcagaagcaggcaaaagtgctggacagctatatgaagagtccctcggaaaggttgtagaagaattaaaaaggactaactgcagctacatactaaataaatatgacacctactcaacgaaggagtatctaattaaagaaggagatctgagtcctggagctgtagatatgattggagacctactgaatgaagattctggctattatgtgtcttttattgagagcctgaaacatgatgatatcttcgcttatgaaaaaagatttgatgaaattgttgatggaatggataagttgcctacagccatgtatcgagacattcaggataaggtgcatttcaatgcccaagtaatcaagatacagcaaaatgaccagaaagtcacagtggtatatgaaaccttatcaaaggagacgccatctgtgacagctgattatgtcattgtgtgcactacgtcaagggccgtccgtctcatcaaatttaatccaccccttttgccaaagaaagcgcatgctttgcggtctgtccactatagaagtggcaccaagatcttcctcacttgcactacgaaattttgggaggatgatggcattcatggtgggaagtccacaactgatcttccatcccgattcatctactaccctaaccataactttactaatggagttggggttattatagcctatggcattggtgatgatgccaatttctttcaagctcttgatttcaaggactgtgctgatattgtctttaatgacctttcattgatccatcagctgccgaagaaagatatccagtccttctgttatccctcagtgattcaaaaatggagcctggataagtatgctatgggtggtataaccaccttcactccctaccagtttcaacattttagtgacccactcactgcatctcaaggcagaatctactttgcaggggagtatacggcccaagctcatggttggattgacagcacaattaagtcagggctgagagcagcaagagatgtgaatcttgcttctgagaatccatcaggaatccacctgagcaatgacaatgaactttaagaaggaggtcagcaatgtttcagactaaaattcccagaataccagttggtaattctaagagctatagtcccaggaggttggagccagaaggaaggggggaagtaagctgatttggcctggctaaattatcaaaagtgatctatcagccaaaacatgtcaaaatatatcagagcattttagaggaaacaaaaataaaccacagaatgaaaaggattcttagcatgccaccccaatttggaaaggaagtcctctcaagacaatctatatatactactaaaactctcttttctgtaacattttactggacaaaacagtgcaccatagggcaagaatgtttgaaccaaaatacctgagctgatgttttattgtgctgtacagttcgccatgggctattttcaaggcagatacatctgtgaatgtatcccaacttttcagtcaagatagcacattatttttgctctgccgttgtttaaatgattgaggaatctggtaaagaattatctctgaagggatgaccagaaagtcatggattgtctagtgctgcaataaagataggatatttctgttgcccattccaaatagtaatggaaagtaatggaaaagaaatgtgcaattgttgttaaactgactttgtgagtttattttaatgtatgactatatttctttgggacatcaagcatctagtggtcctaagctatgattctatatttcctacaaccatcaaaaattgtcagtgctacagtgtcactgatggagaagaaaacacattatggcatcaagcaggcctgtagacaaccttaatttgtaatttctccagtttgccaccctcctctgactagtgttctgaaaaactggggatacagataattataataaaagcacatattatcaaaaaaaaaaaaaaaaaaaaa

SEQ ID NO: 14 - protein, 516 aa, Calloselasma rhodostoma
MNVFFMFSLLFLAALGSCADDRNPLAECFQENDYEEFLEIARNGLKATSNPKHVVIVGAGMAGLSAAYVLAGAGHQVTVLEASERPGGRVRTYRNEEAGWYANLGPMRLPEKHRIVREYIRKFDLRLNEFSQENDNAWYFIKNIRKKVGEVKKDPGLLKYPVKPSEAGKSAGQLYEESLGKVVEELKRTNCSYILNKYDTYSTKEYLIKEGDLSPGAVDMIGDLLNEDSGYYVSFIESLKHDDIFAYEKRFDEIVDGMDKLPTAMYRDIQDKVHFNAQVIKIQQNDQKVTVVYETLSKETPSVTADYVIVCTTSRAVRLIKFNPPLLPKKAHALRSVHYRSGTKIFLTCTTKFWEDDGIHGGKSTTDLPSRFIYYPNHNFTNGVGVIIAYGIGDDANFFQALDFKDCADIVFNDLSLIHQLPKKDIQSFCYPSVIQKWSLDKYAMGGITTFTPYQFQHFSDPLTASQGRIYFAGEYTAQAHGWIDSTIKSGLRAARDVNLASENPSGIHLSNDNEL

SEQ ID NO: 15 - DNA, 1605 bp, Rhodococcus opacus
ttggcattcacacgtagatctttcatgaagggcctcggggccaccggcggcgcaggcctcgcgtacggcgcgatgtcgacgctcgggctcgcaccgtcgaccgctgcgcccgcccgcaccttccagccgctcgcggccggcgacctgatcggcaaggtgaagggcagccattccgtggtcgtgctcggcggcggccccgccggtctgtgttcggcattcgaactgcagaaggccgggtacaaggtgacggtcctcgaggcccgcacccggcccggtggccgcgtctggaccgcacggggcggcagcgaggagaccgacctgagcggcgagacgcagaagtgcacgttctcggagggccacttctacaacgtcggcgccacccgcatcccgcagagccacatcacgctcgactactgccgcgaactcggcgtcgagatccagggattcggaaaccagaacgccaacacgttcgtgaactaccagagcgacacgtcgctgtctggccagtccgtcacctaccgggccgcgaaggccgacacgttcggctacatgtcggaactgctgaagaaggccaccgatcagggtgccctggatcaggtactgagccgggaggacaaggatgcgctgtcggagttcctcagcgacttcggtgacctgtccgacgacggccgctacctcggatcctcgcgtcgcggttacgattccgagcccggagccggcctgaacttcggcaccgagaagaagccgttcgcgatgcaggaagtgatccgcagcggcatcggccgcaacttcagcttcgacttcggctacgaccaggcgatgatgatgttcaccccggtcggcggcatggaccggatctactacgcgttccaggacaggatcggcaccgacaacatcgtcttcggcgccgaggtgacgtcgatgaagaacgtgtccgagggcgtcaccgtcgaatacaccgccggcggctcgaagaagtcgatcaccgccgactacgcgatctgcacgatcccgccgcacctcgtcggacgactgcagaacaatctgcccggcgacgtgctcaccgcgctgaaggcggccaagccgtcgtcgtccggaaagctcggcatcgagtactcgcgccggtggtgggagacggaggaccgcatctacggcggcgcgtccaacaccgacaaggacatctcgcagatcatgttcccgtacgaccactacaactccgatcgcggtgtggtcgtcgcctactacagcagcggcaagcgtcaggaggcgttcgagtccctcacgcaccgccagcggctcgccaaggcgatcgcggagggctcggagatccacggcgagaagtacacccgcgacatctcgtcgtcgttctcgggcagctggcggcgcaccaagtactccgagagtgcctgggccaactgggcgggcagtggcggatcgcacggcggggcggccactcccgagtacgagaagctgctcgaacccgtcgacaagatctatttcgccggcgaccacctgtccaacgccatcgcctggcagcacggcgccctgacgtccgcccgcgacgtcgtcacccacatccacgagcgcgtggcccaggaagcctga

SEQ ID NO: 16 - protein, 534 aa, Rhodococcus opacus
LAFTRRSFMKGLGATGGAGLAYGAMSTLGLAPSTAAPARTFQPLAAGDLIGKVKGSHSVVVLGGGPAGLCSAFELQKAGYKVTVLEARTRPGGRVWTARGGSEETDLSGETQKCTFSEGHFYNVGATRIPQSHITLDYCRELGVEIQGFGNQNANTFVNYQSDTSLSGQSVTYRAAKADTFGYMSELLKKATDQGALDQVLSREDKDALSEFLSDFGDLSDDGRYLGSSRRGYDSEPGAGLNFGTEKKPFAMQEVIRSGIGRNFSFDFGYDQAMMMFTPVGGMDRIYYAFQDRIGTDNIVFGAEVTSMKNVSEGVTVEYTAGGSKKSITADYAICTIPPHLVGRLQNNLPGDVLTALKAAKPSSSGKLGIEYSRRWWETEDRIYGGASNTDKDISQIMFPYDHYNSDRGVVVAYYSSGKRQEAFESLTHRQRLAKAIAEGSEIHGEKYTRDISSSFSGSWRRTKYSESAWANWAGSGGSHGGAATPEYEKLLEPVDKIYFAGDHLSNAIAWQHGALTSARDVVTHIHERVAQEA

SEQ ID NO: 17 - DNA, 1134 bp, Rhizobium leguminosarum
atgagaggaacgcgcatgcgtgtcggttgcccgaaggaaatcaagaatcatgaatatcgcgtcggcctgacgccggcttcggtgcgcgaatatgttgcccacggccacgaggtctgggtggagaccaaggcgggcgtcggtatcggcgctgatgatgccgcctatgccgcggctggcgccaagatcgccgcctccgccaaggatatcttcgaaaagtgcgacatgatcgtcaaggtgaaggagccgcagcctgccgaatgggcgcagctccgcgacggtcagcttctctacacctatctgcatctggcgccggatcccgaacagaccaaaggcctcatcgcctccggcgtcaccgcgatcgcctatgagacggtgaccgacgagcgcggcggcctgccgttgctggcgccgatgtcggaggtcgccggtcgcctgtcgatccaggcaggagcgaccgccctgcagaaggccaatggcggcctcggcgtcctcctcggcggcgtgcccggcgtgctgccggccaaggtcgcagtcatcggcggcggcgtcgtcggcctgcatgcggccaggatggccgccggccttggcgccgatgtcagcatccttgacaagtcgctgccgcgtctgcgccagctcgacgatatctttgcaggccgcatccacacccgttattccagcatccaggcgctggaggaggaagtcttctcggccgatctcattatcggcgccgtgctgatcccgggcgctgccgccccgaagctcgtcacccgcgagatgctgtccggcatgaagaagggctccgtcatcgtcgacgttgccatcgaccagggcggctgcttcgagacctcgcatgcgacgacccattccgatccgacctatgaagtcgatggcgtcgtgcattattgcgttgccaacatgccgggcgccgtgccggtcacctcggcacacgcgctgaacaatgccacattggttcatggcctggcgcttgccgatcgcggcctgcgcgccatcgccgaagacaggcatctgaggaatggcctcaacgtccacaagggccgcatcaccagcaagccggtcgccgaagcgctgggctacgaggccttcgcgccggaaagcgtgctgaacgtagcgtaa

SEQ ID NO: 18 - protein, 377 aa, Rhizobium leguminosarum
MRGTRMRVGCPKEIKNHEYRVGLTPASVREYVAHGHEVWVETKAGVGIGADDAAYAAAGAKIAASAKDIFEKCDMIVKVKEPQPAEWAQLRDGQLLYTYLHLAPDPEQTKGLIASGVTAIAYETVTDERGGLPLLAPMSEVAGRLSIQAGATALQKANGGLGVLLGGVPGVLPAKVAVIGGGVVGLHAARMAAGLGADVSILDKSLPRLRQLDDIFAGRIHTRYSSIQALEEEVFSADLIIGAVLIPGAAAPKLVTREMLSGMKKGSVIVDVAIDQGGCFETSHATTHSDPTYEVDGVVHYCVANMPGAVPVTSAHALNNATLVHGLALADRGLRAIAEDRHLRNGLNVHKGRITSKPVAEALGYEAFAPESVLNVA

SEQ ID NO: 19 - DNA, 1116 bp, Streptomyces coelicolor
gtgaaggtcggcatcccccgcgaggtcaagaacaacgagttccgggtggccatcacccccgccggcgtgcacgagctggtgcgccacggtcaccaggtcgtcgtcgagcgcaacgccggcgtcggctcctcgatccccgacgaggagtacgtcacggccggtgcgcggatcctcgacaccgccgacgaggtctgggccaccgcggacctgctcctgaaggtcaaggagccgatcgcggaggagtaccaccgcctgcgcaaggaccagacgctcttcacctacctgcacctggccgcctccaaggagtgcacggacgcgctcatcgagtcccgcaccaccgccatcgcgtacgagacggtcgagctgcccagccgcgcgctgccgctgctggccccgatgtccgaggtcgcgggccgcctcgccccccaggtcggcgcctaccacctgatggccgccaacggcgggcgcggtgtgctgcccggcggtgtccccggtgtgctcgcgggccgcgccgtcgtcatcggcggcggtgtctccggctggaacgcggcgcagatcgccatcggcctgggcttccacgtcaccctgctcgacaaggacatcaccaagctcagggaagccgacaagatcttcggcacgaagatccagaccgtcgtctccaacgccttcgagctggagaaggcctgcctggaggccgacctcgtgatcggcgccgtgctcatcccgggcgccaaggcaccgaagctggtcaccaacgagctggtgtcccggatgaagcccggaagtgtccttgtcgacatcgcgatcgaccagggcggctgcttcgaggactcccacccgaccacccacgccgagccgaccttcccggtccacaactcggtcttctactgcgtcgccaacatgcccggcgcggtgcccaacacctccacctacgcgctgaccaacgccacgctgccgtacatcgtggagctggccgaccgcggctgggccgaggcgctgcgccgcgaccccgcgctggccaagggtctcaacacccatgacggcaaggtcgtttaccgggaggtcgccgaggcacacggcctggagcacgtggagctcgcctcgctgctcgcctaa

SEQ ID NO: 20 - protein, 371 aa, Streptomyces coelicolor
VKVGIPREVKNNEFRVAITPAGVHELVRHGHQVVVERNAGVGSSIPDEEYVTAGARILDTADEVWATADLLLKVKEPIAEEYHRLRKDQTLFTYLHLAASKECTDALIESRTTAIAYETVELPSRALPLLAPMSEVAGRLAPQVGAYHLMAANGGRGVLPGGVPGVLAGRAVVIGGGVSGWNAAQIAIGLGFHVTLLDKDITKLREADKIFGTKIQTVVSNAFELEKACLEADLVIGAVLIPGAKAPKLVTNELVSRMKPGSVLVDIAIDQGGCFEDSHPTTHAEPTFPVHNSVFYCVANMPGAVPNTSTYALTNATLPYIVELADRGWAEALRRDPALAKGLNTHDGKVVYREVAEAHGLEHVELASLLA

SEQ ID NO: 21 - DNA, 993 bp, Bacillus subtilis
atgagtacaaaccgacatcaagcactagggctgactgatcaggaagccgttgatatgtatagaaccatgctgttagcaagaaaaatcgatgaaagaatgtggctgttaaaccgttctggcaaaattccatttgtaatctcttgtcaaggacaggaagcagcacaggtaggagcggctttcgcacttgaccgtgaaatggattatgtattgccgtactacagagacatgggtgtcgtgctcgcgtttggcatgacagcaaaggacttaatgatgtccgggtttgcaaaagcagcagatccgaactcaggaggccgccagatgccgggacatttcggacaaaagaaaaaccgcattgtgacgggatcatctccggttacaacgcaagtgccgcacgcagtcggtattgcgcttgcgggacgtatggagaaaaaggatatcgcagcctttgttacattcggggaagggtcttcaaaccaaggcgatttccatgaaggggcaaactttgccgctgtccataagctgccggttattttcatgtgtgaaaacaacaaatacgcaatctcagtgccttacgataagcaagtcgcatgtgagaacatttccgaccgtgccataggctatgggatgcctggcgtaactgtgaatggaaatgatccgctggaagtttatcaagcggttaaagaagcacgcgaaagggcacgcagaggagaaggcccgacattaattgaaacgatttcttaccgccttacaccacattccagtgatgacgatgacagcagctacagaggccgtgaagaagtagaggaagcgaaaaaaagtgatcccctgcttacttatcaagcttacttaaaggaaacaggcctgctgtccgatgagatagaacaaaccatgctggatgaaattatggcaatcgtaaatgaagcgacggatgaagcggagaacgccccatatgcagctcctgagtcagcgcttgattatgtttatgcgaagtag

SEQ ID NO: 22 - protein, 330 aa, Bacillus subtilis
MSTNRHQALGLTDQEAVDMYRTMLLARKIDERMWLLNRSGKIPFVISCQGQEAAQVGAAFALDREMDYVLPYYRDMGVVLAFGMTAKDLMMSGFAKAADPNSGGRQMPGHFGQKKNRIVTGSSPVTTQVPHAVGIALAGRMEKKDIAAFVTFGEGSSNQGDFHEGANFAAVHKLPVIFMCENNKYAISVPYDKQVACENISDRAIGYGMPGVTVNGNDPLEVYQAVKEARERARRGEGPTLIETISYRLTPHSSDDDDSSYRGREEVEEAKKSDPLLTYQAYLKETGLLSDEIEQTMLDEIMAIVNEATDEAENAPYAAPESALDYVYAK

SEQ ID NO: 23 - DNA, 984 bp, Bacillus subtilis
atgtcagtaatgtcatatattgatgcaatcaatttggcgatgaaagaagaaatggaacgagattctcgcgttttcgtccttggggaagatgtaggaagaaaaggcggtgtgtttaaagcgacagcgggactctatgaacaatttggggaagagcgcgttatggatacgccgcttgctgaatctgcaatcgcaggagtcggtatcggagcggcaatgtacggaatgagaccgattgctgaaatgcagtttgctgatttcattatgccggcagtcaaccaaattatttctgaagcggctaaaatccgctaccgcagcaacaatgactggagctgtccgattgtcgtcagagcgccatacggcggaggcgtgcacggagccctgtatcattctcaatcagtcgaagcaattttcgccaaccagcccggactgaaaattgtcatgccatcaacaccatatgacgcgaaagggctcttaaaagccgcagttcgtgacgaagaccccgtgctgttttttgagcacaagcgggcataccgtctgataaagggcgaggttccggctgatgattatgtcctgccaatcggcaaggcggacgtaaaaagggaaggcgacgacatcacagtgatcacatacggcctgtgtgtccacttcgccttacaagctgcagaacgtctcgaaaaagatggcatttcagcgcatgtggtggatttaagaacagtttacccgcttgataaagaagccatcatcgaagctgcgtccaaaactggaaaggttcttttggtcacagaagatacaaaagaaggcagcatcatgagcgaagtagccgcaattatatccgagcattgtctgttcgacttagacgcgccgatcaaacggcttgcaggtcctgatattccggctatgccttatgcgccgacaatggaaaaatactttatggtcaaccctgataaagtggaagcggcgatgagagaattagcggagttttaa

SEQ ID NO: 24 - protein, 327 aa, Bacillus subtilis
MSVMSYIDAINLAMKEEMERDSRVFVLGEDVGRKGGVFKATAGLYEQFGEERVMDTPLAESAIAGVGIGAAMYGMRPIAEMQFADFIMPAVNQIISEAAKIRYRSNNDWSCPIVVRAPYGGGVHGALYHSQSVEAIFANQPGLKIVMPSTPYDAKGLLKAAVRDEDPVLFFEHKRAYRLIKGEVPADDYVLPIGKADVKREGDDITVITYGLCVHFALQAAERLEKDGISAHVVDLRTVYPLDKEAIIEAASKTGKVLLVTEDTKEGSIMSEVAAIISEHCLFDLDAPIKRLAGPDIPAMPYAPTMEKYFMVNPDKVEAAMRELAEF

SEQ ID NO: 25 - DNA, 1275 bp, Bacillus subtilis
atggcaattgaacaaatgacgatgccgcagcttggagaaagcgtaacagaggggacgatcagcaaatggcttgtcgcccccggtgataaagtgaacaaatacgatccgatcgcggaagtcatgacagataaggtaaatgcagaggttccgtcttcttttactggtacgataacagagcttgtgggagaagaaggccaaaccctgcaagtcggagaaatgatttgcaaaattgaaacagaaggcgcgaatccggctgaacaaaaacaagaacagccagcagcatcagaagccgctgagaaccctgttgcaaaaagtgctggagcagccgatcagcccaataaaaagcgctactcgccagctgttctccgtttggccggagagcacggcattgacctcgatcaagtgacaggaactggtgccggcgggcgcatcacacgaaaagatattcagcgcttaattgaaacaggcggcgtgcaagaacagaatcctgaggagctgaaaacagcagctcctgcaccgaagtctgcatcaaaacctgagccaaaagaagagacgtcatatcctgcgtctgcagccggtgataaagaaatccctgtcacaggtgtaagaaaagcaattgcttccaatatgaagcgaagcaaaacagaaattccgcatgcttggacgatgatggaagtcgacgtcacaaatatggttgcatatcgcaacagtataaaagattcttttaagaagacagaaggctttaatttaacgttcttcgccttttttgtaaaagcggtcgctcaggcgttaaaagaattcccgcaaatgaatagcatgtgggcgggggacaaaattattcagaaaaaggatatcaatatttcaattgcagttgccacagaggattctttatttgttccggtgattaaaaacgctgatgaaaaaacaattaaaggcattgcgaaagacattaccggcctagctaaaaaagtaagagacggaaaactcactgcagatgacatgcagggaggcacgtttaccgtcaacaacacaggttcgttcgggtctgttcagtcgatgggcattatcaactaccctcaggctgcgattcttcaagtagaatccatcgtcaaacgcccggttgtcatggacaatggcatgattgctgtcagagacatggttaatctgtgcctgtcattagatcacagagtgcttgacggtctcgtgtgcggacgattcctcggacgagtgaaacaaattttagaatcgattgacgagaagacatctgtttactaa

SEQ ID NO: 26 - protein, 424 aa, Bacillus subtilis
MAIEQMTMPQLGESVTEGTISKWLVAPGDKVNKYDPIAEVMTDKVNAEVPSSFTGTITELVGEEGQTLQVGEMICKIETEGANPAEQKQEQPAASEAAENPVAKSAGAADQPNKKRYSPAVLRLAGEHGIDLDQVTGTGAGGRITRKDIQRLIETGGVQEQNPEELKTAAPAPKSASKPEPKEETSYPASAAGDKEIPVTGVRKAIASNMKRSKTEIPHAWTMMEVDVTNMVAYRNSIKDSFKKTEGFNLTFFAFFVKAVAQALKEFPQMNSMWAGDKIIQKKDINISIAVATEDSLFVPVIKNADEKTIKGIAKDITGLAKKVRDGKLTADDMQGGTFTVNNTGSFGSVQSMGIINYPQAAILQVESIVKRPVVMDNGMIAVRDMVNLCLSLDHRVLDGLVCGRFLGRVKQILESIDEKTSVY

SEQ ID NO: 27 - DNA, 1413 bp, Bacillus subtilis
atggtagtaggagatttccctattgaaacagatactcttgtaattggtgcgggacctggcggctatgtagctgccatccgcgctgcacagcttggacaaaaagtaacagtcgttgaaaaagcaactcttggaggcgtttgtctgaacgttggatgtatcccttcaaaagcgctgatcaatgcaggtcaccgttatgagaatgcaaaacattctgatgacatgggaatcactgctgagaatgtaacagttgatttcacaaaagttcaagaatggaaagcttctgttgtcaacaagcttactggcggtgtagcaggtcttcttaaaggcaacaaagtagatgttgtaaaaggtgaagcttactttgtagacagcaattcagttcgtgttatggatgagaactctgctcaaacatacacgtttaaaaacgcaatcattgctactggttctcgtcctatcgaattgccaaacttcaaatatagtgagcgtgtcctgaattcaactggcgctttggctcttaaagaaattcctaaaaagctcgttgttatcggcggcggatacatcggaactgaacttggaactgcgtatgctaacttcggtactgaacttgttattcttgaaggcggagatgaaattcttcctggcttcgaaaaacaaatgagttctctcgttacacgcagactgaagaaaaaaggcaacgttgaaatccatacaaacgcgatggctaaaggcgttgaagaaagaccagacggcgtaacagttactttcgaagtaaaaggcgaagaaaaaactgttgatgctgattacgtattgattacagtaggacgccgtccaaacactgatgagcttggtcttgagcaagtcggtatcgaaatgacggaccgcggtatcgtgaaaactgacaaacagtgccgcacaaacgtacctaacatttatgcaatcggtgatatcatcgaaggaccgccgcttgctcataaagcatcttacgaaggtaaaatcgctgcagaagctatcgctggagagcctgcagaaatcgattaccttggtattcctgcggttgttttctctgagcctgaacttgcatcagttggttacactgaagcacaggcgaaagaagaaggtcttgacattgttgctgctaaattcccatttgcagcaaacggccgcgcgctttctcttaacgaaacagacggcttcatgaagctgatcactcgtaaagaggacggtcttgtgatcggtgcgcaaatcgccggagcaagtgcttctgatatgatttctgaattaagcttagcgattgaaggcggcatgactgctgaagatatcgcaatgacaattcacgctcacccaacattgggcgaaatcacaatggaagctgctgaagtggcaatcggaagtccgattcacatcgtaaaataa

SEQ ID NO: 28 - protein, 470 aa, Bacillus subtilis
MVVGDFPIETDTLVIGAGPGGYVAAIRAAQLGQKVTVVEKATLGGVCLNVGCIPSKALINAGHRYENAKHSDDMGITAENVTVDFTKVQEWKASVVNKLTGGVAGLLKGNKVDVVKGEAYFVDSNSVRVMDENSAQTYTFKNAIIATGSRPIELPNFKYSERVLNSTGALALKEIPKKLVVIGGGYIGTELGTAYANFGTELVILEGGDEILPGFEKQMSSLVTRRLKKKGNVEIHTNAMAKGVEERPDGVTVTFEVKGEEKTVDADYVLITVGRRPNTDELGLEQVGIEMTDRGIVKTDKQCRTNVPNIYAIGDIIEGPPLAHKASYEGKIAAEAIAGEPAEIDYLGIPAVVFSEPELASVGYTEAQAKEEGLDIVAAKFPFAANGRALSLNETDGFMKLITRKEDGLVIGAQIAGASASDMISELSLAIEGGMTAEDIAMTIHAHPTLGEITMEAAEVAIGSPIHIVK

SEQ ID NO: 29 - DNA, 2664 bp, Escherichia coli
atgtcagaacgtttcccaaatgacgtggatccgatcgaaactcgcgactggctccaggcgatcgaatcggtcatccgtgaagaaggtgttgagcgtgctcagtatctgatcgaccaactgcttgctgaagcccgcaaaggcggtgtaaacgtagccgcaggcacaggtatcagcaactacatcaacaccatccccgttgaagaacaaccggagtatccgggtaatctggaactggaacgccgtattcgttcagctatccgctggaacgccatcatgacggtgctgcgtgcgtcgaaaaaagacctcgaactgggcggccatatggcgtccttccagtcttccgcaaccatttatgatgtgtgctttaaccacttcttccgtgcacgcaacgagcaggatggcggcgacctggtttacttccagggccacatctccccgggcgtgtacgctcgtgctttcctggaaggtcgtctgactcaggagcagctggataacttccgtcaggaagttcacggcaatggcctctcttcctatccgcacccgaaactgatgccggaattctggcagttcccgaccgtatctatgggtctgggtccgattggtgctatttaccaggctaaattcctgaaatatctggaacaccgtggcctgaaagatacctctaaacaaaccgtttacgcgttcctcggtgacggtgaaatggacgaaccggaatccaaaggtgcgatcaccatcgctacccgtgaaaaactggataacctggtcttcgttatcaactgtaacctgcagcgtcttgacggcccggtcaccggtaacggcaagatcatcaacgaactggaaggcatcttcgaaggtgctggctggaacgtgatcaaagtgatgtggggtagccgttgggatgaactgctgcgtaaggataccagcggtaaactgatccagctgatgaacgaaaccgttgacggcgactaccagaccttcaaatcgaaagatggtgcgtacgttcgtgaacacttcttcggtaaatatcctgaaaccgcagcactggttgcagactggactgacgagcagatctgggcactgaaccgtggtggtcacgatccgaagaaaatctacgctgcattcaagaaagcgcaggaaaccaaaggcaaagcgacagtaatccttgctcataccattaaaggttacggcatgggcgacgcggctgaaggtaaaaacatcgcgcaccaggttaagaaaatgaacatggacggtgtgcgtcatatccgcgaccgtttcaatgtgccggtgtctgatgcagatatcgaaaaactgccgtacatcaccttcccggaaggttctgaagagcatacctatctgcacgctcagcgtcagaaactgcacggttatctgccaagccgtcagccgaacttcaccgagaagcttgagctgccgagcctgcaagacttcggcgcgctgttggaagagcagagcaaagagatctctaccactatcgctttcgttcgtgctctgaacgtgatgctgaagaacaagtcgatcaaagatcgtctggtaccgatcatcgccgacgaagcgcgtactttcggtatggaaggtctgttccgtcagattggtatttacagcccgaacggtcagcagtacaccccgcaggaccgcgagcaggttgcttactataaagaagacgagaaaggtcagattctgcaggaagggatcaacgagctgggcgcaggttgttcctggctggcagcggcgacctcttacagcaccaacaatctgccgatgatcccgttctacatctattactcgatgttcggcttccagcgtattggcgatctgtgctgggcggctggcgaccagcaagcgcgtggcttcctgatcggcggtacttccggtcgtaccaccctgaacggcgaaggtctgcagcacgaagatggtcacagccacattcagtcgctgactatcccgaactgtatctcttacgacccggcttacgcttacgaagttgctgtcatcatgcatgacggtctggagcgtatgtacggtgaaaaacaagagaacgtttactactacatcactacgctgaacgaaaactaccacatgccggcaatgccggaaggtgctgaggaaggtatccgtaaaggtatctacaaactcgaaactattgaaggtagcaaaggtaaagttcagctgctcggctccggttctatcctgcgtcacgtccgtgaagcagctgagatcctggcgaaagattacggcgtaggttctgacgtttatagcgtgacctccttcaccgagctggcgcgtgatggtcaggattgtgaacgctggaacatgctgcacccgctggaaactccgcgcgttccgtatatcgctcaggtgatgaacgacgctccggcagtggcatctaccgactatatgaaactgttcgctgagcaggtccgtacttacgtaccggctgacgactaccgcgtactgggtactgatggcttcggtcgttccgacagccgtgagaacctgcgtcaccacttcgaagttgatgcttcttatgtcgtggttgcggcgctgggcgaactggctaaacgtggcgaaatcgataagaaagtggttgctgacgcaatcgccaaattcaacatcgatgcagataaagttaacccgcgtctggcgtaa

SEQ ID NO: 30 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 31 - DNA, 1893 bp, Escherichia coli
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaa

SEQ ID NO: 32 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMVFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 33 - DNA, 1425 bp, Escherichia coli
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 34 - protein, 474 aa, Escherichia coli [sequence omitted]

SEQ ID NO: 35 - DNA, 2802 bp, Escherichia coli
atgcagaacagcgctttgaaagcctggttggactcttcttacctctctggcgcaaaccagagctggatagaacagctctatgaagacttcttaaccgatcctgactcggttgacgctaactggcgttcgacgttccagcagttacctggtacgggagtcaaaccggatcaattccactctcaaacgcgtgaatatttccgccgcctggcgaaagacgcttcacgttactcttcaacgatctccgaccctgacaccaatgtgaagcaggttaaagtcctgcagctcattaacgcataccgcttccgtggtcaccagcatgcgaatctcgatccgctgggactgtggcagcaagataaagtggccgatctggatccgtctttccacgatctgaccgaagcagacttccaggagaccttcaacgtcggttcatttgccagcggcaaagaaaccatgaaactcggcgagctgctggaagccctcaagcaaacctactgcggcccgattggtgccgagtatatgcacattaccagcaccgaagaaaaacgctggatccaacagcgtatcgagtctggtcgcgcgactttcaatagcgaagagaaaaaacgcttcttaagcgaactgaccgccgctgaaggtcttgaacgttacctcggcgcaaaattccctggcgcaaaacgcttctcgctggaaggcggtgacgcgttaatcccgatgcttaaagagatgatccgccacgctggcaacagcggcacccgcgaagtggttctcgggatggcgcaccgtggtcgtctgaacgtgctggtgaacgtgctgggtaaaaaaccgcaagacttgttcgacgagttcgccggtaaacataaagaacacctcggcacgggtgacgtgaaataccacatgggcttctcgtctgacttccagaccgatggcggcctggtgcacctggcgctggcgtttaacccgtctcaccttgagattgtaagcccggtagttatcggttctgttcgtgcccgtctggacagacttgatgagccgagcagcaacaaagtgctgccaatcaccatccacggtgacgccgcagtgaccgggcagggcgtggttcaggaaaccctgaacatgtcgaaagcgcgtggttatgaagttggcggtacggtacgtatcgttatcaacaaccaggttggtttcaccacctctaatccgctggatgcccgttctacgccgtactgtactgatatcggtaagatggttcaggccccgattttccacgttaacgcggacgatccggaagccgttgcctttgtgacccgtctggcgctcgatttccgtaacacctttaaacgtgatgtcttcatcgacctggtgtgctaccgccgtcacggccacaacgaagccgacgagccgagcgcaacccagccgctgatgtatcagaaaatcaaaaaacatccgacaccgcgcaaaatctacgctgacaagctggagcaggaaaaagtggcgacgctggaagatgccaccgagatggttaacctgtaccgcgatgcgctggatgctggcgattgcgtagtggcagagtggcgtccgatgaacatgcactctttcacctggtcgccgtacctcaaccacgaatgggacgaagagtacccgaacaaagttgagatgaagcgcctgcaggagctggcgaaacgcatcagcacggtgccggaagcagttgaaatgcagtctcgcgttgccaagatttatggcgatcgccaggcgatggctgccggtgagaaactgttcgactggggcggtgcggaaaacctcgcttacgccacgctggttgatgaaggcattccggttcgcctgtcgggtgaagactccggtcgcggtaccttcttccaccgccacgcggtgatccacaaccagtctaacggttccacttacacgccgctgcaacatatccataacgggcagggcgcgttccgtgtctgggactccgtactgtctgaagaagcagtgctggcgtttgaatatggttatgccaccgcagaaccacgcactctgaccatctgggaagcgcagttcggtgacttcgccaacggtgcgcaggtggttatcgaccagttcatctcctctggcgaacagaaatggggccggatgtgtggtctggtgatgttgctgccgcacggttacgaagggcaggggccggagcactcctccgcgcgtctggaacgttatctgcaactttgtgctgagcaaaacatgcaggtttgcgtaccgtctaccccggcacaggtttaccacatgctgcgtcgtcaggcgctgcgcgggatgcgtcgtccgctggtcgtgatgtcgccgaaatccctgctgcgtcatccgctggcggtttccagcctcgaagaactggcgaacggcaccttcctgccagccatcggtgaaatcgacgagcttgatccgaagggcgtgaagcgcgtagtgatgtgttctggtaaggtttattacgacctgctggaacagcgtcgtaagaacaatcaacacgatgtcgccattgtgcgtatcgagcaactctacccgttcccgcataaagcgatgcaggaagtgttgcagcagtttgctcacgtcaaggattttgtctggtgccaggaagagccgctcaaccagggcgcatggtactgcagccagcatcatttccgtgaagtgattccgtttggggcttctctgcgttatgcaggccgcccggcctccgcctctccggcggtagggtatatgtccgttcaccagaaacagcaacaagatctggttaatgacgcgctgaacgtcgaataa

SEQ ID NO: 36 - protein, 933 aa, Escherichia coli
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 37 - DNA, 1503 bp, Escherichia coli
tcgaataaataaaggatacacaatgagtagcgtagatattctggtccctgacctgcctgaatccgtagccgatgccaccgtcgcaacctggcataaaaaacccggcgacgcagtcgtacgtgatgaagtgctggtagaaatcgaaactgacaaagtggtactggaagtaccggcatcagcagacggcattctggatgcggttctggaagatgaaggtacaacggtaacgtctcgtcagatccttggtcgcctgcgtgaaggcaacagcgccggtaaagaaaccagcgccaaatctgaagagaaagcgtccactccggcgcaacgccagcaggcgtctctggaagagcaaaacaacgatgcgttaagcccggcgatccgtcgcctgctggctgaacacaatctcgacgccagcgccattaaaggcaccggtgtgggtggtcgtctgactcgtgaagatgtggaaaaacatctggcgaaagccccggcgaaagagtctgctccggcagcggctgctccggcggcgcaaccggctctggctgcacgtagtgaaaaacgtgtcccgatgactcgcctgcgtaagcgtgtggcagagcgtctgctggaagcgaaaaactccaccgccatgctgaccacgttcaacgaagtcaacatgaagccgattatggatctgcgtaagcagtacggtgaagcgtttgaaaaacgccacggcatccgtctgggctttatgtccttctacgtgaaagcggtggttgaagccctgaaacgttacccggaagtgaacgcttctatcgacggcgatgacgtggtttaccacaactatttcgacgtcagcatggcggtttctacgccgcgcggcctggtgacgccggttctgcgtgatgtcgataccctcggcatggcagacatcgagaagaaaatcaaagagctggcagtcaaaggccgtgacggcaagctgaccgttgaagatctgaccggtggtaacttcaccatcaccaacggtggtgtgttcggttccctgatgtctacgccgatcatcaacccgccgcagagcgcaattctgggtatgcacgctatcaaagatcgtccgatggcggtgaatggtcaggttgagatcctgccgatgatgtacctggcgctgtcctacgatcaccgtctgatcgatggtcgcgaatccgtgggcttcctggtaacgatcaaagagttgctggaagatccgacgcgtctgctgctggacgtgtagtagtttaagtttcacctgcactgtagaccggataaggcattatcgccttctccggcaattgaagcctgatgcgacgctgacgcgtcttatcaggcctacgggaccaccaatgtaggtcggataaggcgcaacgccgcatccgacaagcgatgcctgatgtgacgtttaacgtgtcttatcaggcctacgggtgaccgacaatgcccggaagcgatacgaaatattcggtctacggtttaaaagataacgattactgaaggatg

SEQ ID NO: 38 - protein, 405 aa, Escherichia coli
MSSVDILVPDLPESVADATVATWHKKPGDAVVRDEVLVEIETDKVVLEVPASADGILDAVLEDEGTTVTSRQILGRLREGNSAGKETSAKSEEKASTPAQRQQASLEEQNNDALSPAIRRLLAEHNLDASAIKGTGVGGRLTREDVEKHLAKAPAKESAPAAAAPAAQPALAARSEKRVPMTRLRKRVAERLLEAKNSTAMLTTFNEVNMKPIMDLRKQYGEAFEKRHGIRLGFMSFYVKAVVEALKRYPEVNASIDGDDVVYHNYFDVSMAVSTPRGLVTPVLRDVDTLGMADIEKKIKELAVKGRDGKLTVEDLTGGNFTITNGGVFGSLMSTPIINPPQSAILGMHAIKDRPMAVNGQVEILPMMYLALSYDHRLIDGRESVGFLVTIKELLEDPTRLLLDV

SEQ ID NO: 39 - DNA, 1425 bp, Escherichia coli; identical to SEQ ID NO: 33

SEQ ID NO: 40 - protein, 474 aa, Escherichia coli [sequence omitted]

SEQ ID NO: 41 - DNA, 1770 bp, Ralstonia eutropha
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagccggagagctcgctggtgcgccatgtggtggagcagggacatacggtgtttctggtgtcgtggcgcaatccggacgccagcatggccggcagcacctgggacgactacatcgagcacgcggccatccgcgccatcgaagtcgcgcgcgacatcagcggccaggacaagatcaacgtgctcggcttctgcgtgggcggcaccattgtctcgaccgcgctggcggtgctggccgcgcgcggcgagcacccggccgccagcgtcacgctgctgaccacgctgctggactttgccgacacgggcatcctcgacgtctttgtcgacgagggccatgtgcagttgcgcgaggccacgctgggcggcggcgccggcgcgccgtgcgcgctgctgcgcggccttgagctggccaataccttctcgttcttgcgcccgaacgacctggtgtggaactacgtggtcgacaactacctgaagggcaacacgccggtgccgttcgacctgctgttctggaacggcgacgccaccaacctgccggggccgtggtactgctggtacctgcgccacacctacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 42 - protein, 589 aa, Ralstonia eutropha
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 43 - DNA, 642 bp, Escherichia coli
atgaaaaactggaaaacaagtgcagaatcaatcctgaccaccggcccggttgtaccggttatcgtggtaaaaaaactggaacacgcggtgccgatggcaaaagcgttggttgctggtggggtgcgcgttctggaagtgactctgcgtaccgagtgtgcagttgacgctatccgtgctatcgccaaagaagtgcctgaagcgattgtgggtgccggtacggtgctgaatccacagcagctggcagaagtcactgaagcgggtgcacagttcgcaattagcccgggtctgaccgagccgctgctgaaagctgctaccgaagggactattcctctgattccggggatcagcactgtttccgaactgatgctgggtatggactacggtttgaaagagttcaaattcttcccggctgaagctaacggcggcgtgaaagccctgcaggcgatcgcgggtccgttctcccaggtccgtttctgcccgacgggtggtatttctccggctaactaccgtgactacctggcgctgaaaagcgtgctgtgcatcggtggttcctggctggttccggcagatgcgctggaagcgggcgattacgaccgcattactaagctggcgcgtgaagctgtagaaggcgctaagctgtaa

SEQ ID NO: 44 - protein, 213 aa, Escherichia coli
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 45 - DNA, 1575 bp, Clostridium propionicum
atgagaaaggttcccattattaccgcagatgaggctgcaaagcttattaaagacggtgatacagttacaacaagtggtttcgttggaaatgcaatccctgaggctcttgatagagctgtagaaaaaagattcttagaaacaggcgaacccaaaaacattacatatgtttattgtggttctcaaggtaacagagacggaagaggtgctgagcactttgctcatgaaggccttttaaaacgttacatcgctggtcactgggctacagttcctgctttgggtaaaatggctatggaaaataaaatggaagcatataatgtatctcagggtgcattgtgtcatttgttccgtgatatagcttctcataagccaggcgtatttacaaaggtaggtatcggtactttcattgaccccagaaatggcggcggtaaagtaaatgatattaccaaagaagatattgttgaattggtagagattaagggtcaggaatatttattctaccctgcttttcctattcatgtagctcttattcgtggtacttacgctgatgaaagcggaaatatcacatttgagaaagaagttgctcctctggaaggaacttcagtatgccaggctgttaaaaacagtggcggtatcgttgtagttcaggttgaaagagtagtaaaagctggtactcttgaccctcgtcatgtaaaagttccaggaatttatgttgactatgttgttgttgctgacccagaagatcatcagcaatctttagattgtgaatatgatcctgcattatcaggcgagcatagaagacctgaagttgttggagaaccacttcctttgagtgcaaagaaagttattggtcgtcgtggtgccattgaattagaaaaagatgttgctgtaaatttaggtgttggtgcgcctgaatatgtagcaagtgttgctgatgaagaaggtatcgttgattttatgactttaactgctgaaagtggtgctattggtggtgttcctgctggtggcgttcgctttggtgcttcttataatgcggatgcattgatcgatcaaggttatcaattcgattactatgatggcggcggcttagacctttgctatttaggcttagctgaatgcgatgaaaaaggcaatatcaacgtttcaagatttggccctcgtatcgctggttgtggtggtttcatcaacattacacagaatacacctaaggtattcttctgtggtactttcacagcaggtggcttaaaggttaaaattgaagatggcaaggttattattgttcaagaaggcaagcagaaaaaattcttgaaagctgttgagcagattacattcaatggtgacgttgcacttgctaataagcaacaagtaacttatattacagaaagatgcgtattccttttgaaggaagatggtttgcacttatctgaaattgcacctggtattgatttgcagacacagattcttgacgttatggattttgcacctattattgacagagatgcaaacggccaaatcaaattgatggacgctgctttgtttgcagaaggcttaatgggtctgaaggaaatgaagtcctga

SEQ ID NO: 46 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTAESGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 47 - DNA, 780 bp, Metallosphaera sedula
atggaatttgaaacaatagaaactaaaaaagaaggaaacttgttctggattacgttaaatagacccgataaactaaacgcactaaacgctaaattacttgaggagttagatagggcagtctctcaggcagagtctgacccagagattagggttatcatcattacagggaaaggaaaggccttctgcgcaggggctgacataacccagtttaaccagttaaccccagcagaagcctggaaattctctaagaaaggaagagagatcatggacaagatagaggcactgagcaaacccaccattgccatgatcaatggatatgcccttgggggtggactagagctagccttagcctgtgatataaggatcgcagcggaggaggcccaactaggccttccagagataaacctagggatatatccggggtatggggggactcagaggttaaccagagttataggaaagggaagagccctggagatgatgatgacgggcgatcgtattcctggtaaggatgctgagaaatatggtctcgtgaatagggttgtccccctagctaacttggagcaagagacaaggaagctggcagaaaagatagccaagaagtctcctatctctctcgccttaatcaaggaagttgtaaacaggggactagactctcccctactgtcaggtctagcgttggaaagcgtaggatggggagtcgtgttttctacggaggacaagaaggagggggtaagtgccttcctggagaagagagagcctacgtttaagggaaaatag

SEQ ID NO: 48 - protein, 259 aa, Metallosphaera sedula
MEFETIETKKEGNLFWITLNRPDKLNALNAKLLEELDRAVSQAESDPEIRVIIITGKGKAFCAGADITQFNQLTPAEAWKFSKKGREIMDKIEALSKPTIAMINGYALGGGLELALACDIRIAAEEAQLGLPEINLGIYPGYGGTQRLTRVIGKGRALEMMMTGDRIPGKDAEKYGLVNRVVPLANLEQETRKLAEKIAKKSPISLALIKEVVNRGLDSPLLSGLALESVGWGVVFSTEDKKEGVSAFLEKREPTFKGK

SEQ ID NO: 49 - DNA, 2145 bp, Escherichia coli
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 50 - protein, 714 aa, Escherichia coli
VSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 51 - DNA, 1209 bp, Escherichia coli
atgaatgaatttccggttgttttggttattaactgtggttcgtcttcgattaagttttccgtgctcgatgccagcgactgtgaagtattaatgtcaggtattgccgacggtattaactcggaaaatgcattcttatccgtaaatgggggagagccagcaccgctggctcaccacagctacgaaggtgcattgaaggcaattgcatttgaactggaaaaacggaatttaaatgacagtgtggccttaattggccaccgcatcgctcacggcggcagtatttttaccgagtccgccattattaccgatgaagtcattgataatatccgtcgcgtttctccactggcacccctgcataattacgccaatttaagtggtattgaatcggcgcagcaattatttccgggcgtaactcaggtggcggtatttgataccagtttccaccagacgatggctccggaagcttatttatacggcctgccgtggaaatattatgaagagttaggtgtacgccgttatggtttccacggcacgtcgcaccgctatgtttcccagcgcgcacattcgctgctgaatctggcggaagatgactccggcctggttgtggcgcatcttggcaatggcgcgtcaatctgcgcggttcgcaacggtcagagtgttgatacctcaatgggaatgacgccgctggaaggcttgatgatgggtacccgcagtggcgatgtcgactttggtgcgatgtcctgggtcgccagccaaaccaaccagagcctgggtgacctggaacgcgtagtgaataaagagtcgggattattaggtatttccggtctttcttcggatttacgtgttctggaaaaagcctggcatgaaggtcacgaacgcgcgcaactggcaattaaaacctttgttcaccgaattgcccgtcatattgccggacacgcagcttcattacgtcgcctggatggaattatattcaccggcggaataggagagaattcaagcttaattcgtcgtctggtcatggaacatttggctgtattaggcttagagattgatacagaaatgaataatcgctctaactcctgtggtgagcgaattgtttccagtgaaaatgcgcgtgtcatttgtgccgttattccgactaacgaagaaaaaatgattgctttggatgccattcatttaggcaaagttaacgcgcccgcagaatttgcataa

SEQ ID NO: 52 - protein, 402 aa, Escherichia coli
MNEFPVVLVINCGSSSIKFSVLDASDCEVLMSGIADGINSENAFLSVNGGEPAPLAHHSYEGALKAIAFELEKRNLNDSVALIGHRIAHGGSIFTESAIITDEVIDNIRRVSPLAPLHNYANLSGIESAQQLFPGVTQVAVFDTSFHQTMAPEAYLYGLPWKYYEELGVRRYGFHGTSHRYVSQRAHSLLNLAEDDSGLVVAHLGNGASICAVRNGQSVDTSMGMTPLEGLMMGTRSGDVDFGAMSWVASQTNQSLGDLERVVNKESGLLGISGLSSDLRVLEKAWHEGHERAQLAIKTFVHRIARHIAGHAASLRRLDGIIFTGGIGENSSLIRRLVMEHLAVLGLEIDTEMNNRSNSCGERIVSSENARVICAVIPTNEEKMIALDAIHLGKVNAPAEFA

SEQ ID NO: 53 - DNA, 1644 bp, Lactococcus lactis
atgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatag

SEQ ID NO: 54 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 55 - DNA, 1488 bp, Escherichia coli
atgaattttcatcatctggcttactggcaggataaagcgttaagtctcgccattgaaaaccgcttatttattaacggtgaatatactgctgcggcggaaaatgaaacctttgaaaccgttgatccggtcacccaggcaccgctggcgaaaattgcccgcggcaagagcgtcgatatcgaccgtgcgatgagcgcagcacgcggcgtatttgaacgcggcgactggtcactctcttctccggctaaacgtaaagcggtactgaataaactcgccgatttaatggaagcccacgccgaagagctggcactgctggaaactctcgacaccggcaaaccgattcgtcacagtctgcgtgatgatattcccggcgcggcgcgcgccattcgctggtacgccgaagcgatcgacaaagtgtatggcgaagtggcgaccaccagtagccatgagctggcgatgatcgtgcgtgaaccggtcggcgtgattgccgccatcgtgccgtggaacttcccgctgttgctgacttgctggaaactcggcccggcgctggcggcgggaaacagcgtgattctaaaaccgtctgaaaaatcaccgctcagtgcgattcgtctcgcggggctggcgaaagaagcaggcttgccggatggtgtgttgaacgtggtgacgggttttggtcatgaagccgggcaggcgctgtcgcgtcataacgatatcgacgccattgcctttaccggttcaacccgtaccgggaaacagctgctgaaagatgcgggcgacagcaacatgaaacgcgtctggctggaagcgggcggcaaaagcgccaacatcgttttcgctgactgcccggatttgcaacaggcggcaagcgccaccgcagcaggcattttctacaaccagggacaggtgtgcatcgccggaacgcgcctgttgctggaagagagcatcgccgatgaattcttagccctgttaaaacagcaggcgcaaaactggcaaccgggccatccacttgatcccgcaaccaccatgggcaccttaatcgactgcgcccacgccgactcggtccatagctttattcgggaaggcgaaagcaaagggcaactgttgttggatggccgtaacgccgggctggctgccgccatcggcccgaccatctttgtggatgtggacccgaatgcgtccttaagtcgcgaagagattttcggtccggtgctggtggtcacgcgtttcacatcagaagaacaggcgctacagcttgccaacgacagccagtacggccttggcgcggcggtatggacgcgcgacctctcccgcgcgcaccgcatgagccgacgcctgaaagccggttccgtcttcgtcaataactacaacgacggcgatatgaccgtgccgtttggcggctataagcagagcggcaacggtcgcgacaaatccctgcatgcccttgaaaaattcactgaactgaaaaccatctggataagcctggaggcctga

SEQ ID NO: 56 - protein, 495 aa, Escherichia coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 57 - DNA, 1491 bp, Klebsiella pneumoniae
atgatgaattttcagcacctggcttactggcaggaaaaagcgaaaaacctggccattgaaacgcgcttatttattaacggcgaatattgcgccgcggccgataataccacctttgagactatcgaccccgccgcgcagcagacattagcccaggtcgcccgcggtaaaaaagccgacgtcgaacgggcggtgaaagccgcgcgccaggcttttgataacggcgactggtcgcaggcctcccccgcacagcgtaaagcgatcctcactcgctttgctaatctgatggaggcccatcgtgaagagctggcgctgctggaaacgctggataccggcaagccgattcgccacagcctgcgcgacgatattcccggcgccgcccgcgccattcgctggtatgccgaagcgctggataaagtctatggcgaagtggcccccaccggcagcaacgagctggcgatgatcgttcgcgaaccaattggcgtgatcgccgcggtggtgccgtggaacttcccgctgctgctggcctgctggaaactcggcccggcgctggcggcaggcaatagcgtaatcctcaaaccctcggaaaaatcgccgcttaccgccctgcgtctggccgggctggcgaaagaggccggcctgccggacggcgtgttgaacgtggtcagcggctttggccacgaggccgggcaggcgctggccctgcatcctgatgttgaagtcatcaccttcaccggctccacccgcaccggcaagcagctgctgaaagacgccggcgacagcaatatgaagcgcgtgtggctggaagcgggcggcaagagcgccaacattgtcttcgccgattgcccggatctgcaacaagcggttcgcgccaccgccggcggcatcttctacaaccagggacaggtgtgcatcgccgggacccgtctgctgctcgaggagagcatcgctgacgagttcctggcgcggctgaaagctgaggcgcaacactggcagccgggcaacccgctcgatccggacaccaccatgggcatgctgattgacaatacccatgccgacaacgtgcatagctttattcgcggcggcgaaagccaaagcaccctgttcctcgacggacggaaaaacccgtggcctgccgccgttggcccgaccattttcgttgacgtcgacccggcatcaaccctcagccgggaagagatcttcggcccggtgctggtggtgacccgcttcaaaagcgaagaagaggcgctaaagctcgccaatgacagcgactacggcttgggcgccgcggtgtggacccgcgatctctcccgcgcccaccgcatgagccgccgcctgaaggccggctcggtcttcgtcaacaactataacgatggtgatatgaccgttccgttcggcggctacaagcagagcggcaacgggcgcgataaatcgctgcacgcgctggaaaaattcaccgaactgaaaaccatctggattgccctggagtcttga

SEQ ID NO: 58 - protein, 496 aa, Klebsiella pneumoniae
MMNFQHLAYWQEKAKNLAIETRLFINGEYCAAADNTTFETIDPAAQQTLAQVARGKKADVERAVKAARQAFDNGDWSQASPAQRKAILTRFANLMEAHREELALLETLDTGKPIRHSLRDDIPGAARAIRWYAEALDKVYGEVAPTGSNELAMIVREPIGVIAAVVPWNFPLLLACWKLGPALAAGNSVILKPSEKSPLTALRLAGLAKEAGLPDGVLNVVSGFGHEAGQALALHPDVEVITFTGSTRTGKQLLKDAGDSNMKRVWLEAGGKSANIVFADCPDLQQAVRATAGGIFYNQGQVCIAGTRLLLEESIADEFLARLKAEAQHWQPGNPLDPDTTMGMLIDNTHADNVHSFIRGGESQSTLFLDGRKNPWPAAVGPTIFVDVDPASTLSREEIFGPVLVVTRFKSEEEALKLANDSDYGLGAAVWTRDLSRAHRMSRRLKAGSVFVNNYNDGDMTVPFGGYKQSGNGRDKSLHALEKFTELKTIWIALES

SEQ ID NO: 59 - DNA, 1395 bp, Salmonella enterica
atgaatacttctgaactcgaaaccctgattcgcaccattcttagcgagcaattaaccacgccggcgcaaacgccggtccagcctcagggcaaagggattttccagtccgtgagcgaggccatcgacgccgcgcaccaggcgttcttacgttatcagcagtgcccgctaaaaacccgcagcgccattatcagcgcgatgcgtcaggagctgacgccgctgctggcgcccctggcggaagagagcgccaatgaaacggggatgggcaacaaagaagataaatttctcaaaaacaaggctgcgctggacaacacgccgggcgtagaagatctcaccaccaccgcgctgaccggcgacggcggcatggtgctgtttgaatactcaccgtttggcgttatcggttcggtcgccccaagcaccaacccgacggaaaccatcatcaacaacagtatcagcatgctggcggcgggcaacagtatctactttagcccgcatccgggagcgaaaaaggtctctctgaagctgattagcctgattgaagagattgccttccgctgctgcggcatccgcaatctggtggtgaccgtggcggaacccaccttcgaagcgacccagcagatgatggcccacccgcgaatcgcagtactggccattaccggcggcccgggcattgtggcaatgggcatgaagagcggtaagaaggtgattggcgctggcgcgggtaacccgccctgcatcgttgatgaaacggcggacctggtgaaagcggcggaagatatcatcaacggcgcgtcattcgattacaacctgccctgcattgccgagaagagcctgatcgtagtggagagtgtcgccgaacgtctggtgcagcaaatgcaaaccttcggcgcgctgctgttaagccctgccgataccgacaaactccgcgccgtctgcctgcctgaaggccaggcgaataaaaaactggtcggcaagagcccatcggccatgctggaagccgccgggatcgctgtccctgcaaaagcgccgcgtctgctgattgcgctggttaacgctgacgatccgtgggtcaccagcgaacagttgatgccgatgctgccagtggtaaaagtcagcgatttcgatagcgcgctggcgctggccctgaaggttgaagaggggctgcatcataccgccattatgcactcgcagaacgtgtcacgcctgaacctcgcggcccgcacgctgcaaacctcgatattcgtcaaaaacggcccctcttatgccgggatcggcgtcggcggcgaaggctttaccaccttcactatcgccacaccaaccggtgaagggaccacgtcagcgcgtacttttgcccgttcccggcgctgcgtactgaccaacggcttttctattcgctaa

SEQ ID NO: 60 - protein, 464 aa, Salmonella enterica
MNTSELETLIRTILSEQLTTPAQTPVQPQGKGIFQSVSEAIDAAHQAFLRYQQCPLKTRSAIISAMRQELTPLLAPLAEESANETGMGNKEDKFLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 61 - DNA, 1386 bp, Klebsiella pneumoniae
atgaatacagcagaactggaaacccttatccgcaccattctcagcgaaaagctcgcgcccgcccccgtttctcaggaacagcagggcatttaccgcgacgtcggcagcgccatcgacgccgcccatcaggcttttctccgctatcagcagtgtccgctaaaaacccgcagcgccattatcagcgccctgcgggagacgctggcccccgagctggcgacgctggcggaggagagcgccacggagaccggcatgggcaacaaagaagataaatatctgaaaaataaagctgcccttgagaacacaccgggcattgaggatctcaccaccagcgccctcaccggcgatggcgggatggtgctgtttgagtactcgccgttcggggttattggcgccgtggcgcccagcaccaacccaacggaaaccattatcaacaacagtatcagcatgctggcggcgggtaacagcgtctatttcagcccccatcccggcgcgaaaaaggtctcgttaacgcttatcgccaggatcgaagagatcgcctaccgctgcagcgggatccgtaacctggtggtgaccgttgccgagccgacctttgaagccacccagcaaatgatggcccacccgctgattgccgttctggctatcaccggcggccctggcattgtggcgatgggcatgaaaagcggtaaaaaagtgatcggcgctggcgccggcaatccgccgtgcatcgttgatgaaacggccgatctcgtcaaagccgccgaagatatcatcagcggcgccgccttcgattacaacctgccctgtatcgccgaaaaaagcctgatcgtcgtcgcctccgtcgctgaccgcctgatccagcagatgcaggattttgacgcgctgctgttgagcccgcaggagaccgataccctgcgcgccgtctgcctgcccgacggcgcggcgaataaaaaactggttggtaagagcccggctgagctgctggcggccgccggtctcgccgtcccttcccgcccccctcgcctgctgatagccgaggtgcaggcgaacgacccctgggtgacctgcgagcaactgatgccggtgctgccgatcgtccgggtcgccgattttgatagcgccctggcgctggccctgcgcgttgaggagggtctgcaccacaccgccattatgcactcgcagaatgtctcgcggctcaatctggcggcacgcacgctgcagacctccatttttgtcaaaaatggtccgtcttacgcgggtatcggcgtcggcggcgaagggtttaccaccttcaccatcgccacgccaaccggagaaggcaccacctccgcgcggacgttcgcccgcctgcggcgctgcgtgttgaccaacggtttttccattcgctaa

SEQ ID NO: 62 - protein, 461 aa, Klebsiella pneumoniae
MNTAELETLIRTILSEKLAPAPVSQEQQGIYRDVGSAIDAAHQAFLRYQQCPLKTRSAIISALRETLAPELATLAEESATETGMGNKEDKYLKNKAALENTPGIEDLTTSALTGDGGMVLFEYSPFGVIGAVAPSTNPTETIINNSISMLAAGNSVYFSPHPGAKKVSLTLIARIEEIAYRCSGIRNLVVTVAEPTFEATQQMMAHPLIAVLAITGGPGIVAMGMKSGKKVIGAGAGNPPCIVDETADLVKAAEDIISGAAFDYNLPCIAEKSLIVVASVADRLIQQMQDFDALLLSPQETDTLRAVCLPDGAANKKLVGKSPAELLAAAGLAVPSRPPRLLIAEVQANDPWVTCEQLMPVLPIVRVADFDSALALALRVEEGLHHTAIMHSQNVSRLNLAARTLQTSIFVKNGPSYAGIGVGGEGFTTFTIATPTGEGTTSARTFARLRRCVLTNGFSIR

SEQ ID NO: 63 - DNA, 1164 bp, Klebsiella pneumoniae
atgagctatcgtatgtttgattatctggtgccaaacgttaacttttttggccccaacgccatttccgtagtcggcgaacgctgccagctgctgggggggaaaaaagccctgctggtcaccgacaaaggcctgcgggcaattaaagatggcgcggtggacaaaaccctgcattatctgcgggaggccgggatcgaggtggcgatctttgacggcgtcgagccgaacccgaaagacaccaacgtgcgtgacggcctcgccgtgtttcgccgcgaacagtgcgacatcatcgtcaccgtgggcggcggcagcccgcacgactgcggtaaaggcatcggcatcgccgccacccatgagggcgatctgtaccagtatgcgggaatcgagaccctgaccaacccgctgccgcctatcgtcgcggtcaacaccactgccggcaccgccagcgaggtcacccgccactgcgtcctgaccaacacccaaaccaaagtgaagtttgtgatcgtcagttggcgcaacctgccatcggtctccatcaacgatccgctgctgatgatcggtaaaccggccgccctgaccgcggcgaccgggatggatgccctgacccacgccgtagaggcctatatctccaaagacgctaacccggtgacggacgccgccgccatgcaggcgatccgcctcatcgcccgcaacctgcgccaggccgtggccctcggcagcaatctgcaggcgcgggaaaacatggcctatgcctctctgctggccgggatggctttcaataacgccaacctcggctacgtgcacgccatggcgcaccagctgggcggcctgtacgacatgccgcacggcgtggccaacgctgtcctgctgccgcatgtggcccgctacaacctgatcgccaatccggagaaattcgccgatatcgctgaactgatgggcgaaaatatcaccggactgtccaccctcgacgcggcggaaaaagccatcgccgctatcacgcgtttgtcgatggatatcggtattccgcagcatctgcgcgatctgggagtaaaagaggccgacttcccctacatggcggagatggctctgaaagacggcaatgcgttctcgaacccgcgtaaaggcaacgagcaggagattgccgcgattttccgccaggcattctga

SEQ ID NO: 64 - protein, 387 aa, Klebsiella pneumoniae
MSYRMFDYLVPNVNFFGPNAISVVGERCQLLGGKKALLVTDKGLRAIKDGAVDKTLHYLREAGIEVAIFDGVEPNPKDTNVRDGLAVFRREQCDIIVTVGGGSPHDCGKGIGIAATHEGDLYQYAGIETLTNPLPPIVAVNTTAGTASEVTRHCVLTNTQTKVKFVIVSWRNLPSVSINDPLLMIGKPAALTAATGMDALTHAVEAYISKDANPVTDAAAMQAIRLIARNLRQAVALGSNLQARENMAYASLLAGMAFNNANLGYVHAMAHQLGGLYDMPHGVANAVLLPHVARYNLIANPEKFADIAELMGENITGLSTLDAAEKAIAAITRLSMDIGIPQHLRDLGVKEADFPYMAEMALKDGNAFSNPRKGNEQEIAAIFRQAF

SEQ ID NO: 65 - DNA, 1158 bp, Clostridium butyricum
atgagaatgtatgattatttagtaccaagtgtaaattttatgggagcaaattcagtatctgtagtaggtgaaagatgcaaaatattaggtggaaagaaagcattgatagttacagataagtttctaaaagatatggaaggtggagctgttgaattaacagttaaatatttaaaagaagctggattagatgctgtatattatgacggagttgaaccaaatccaaaagatgttaatgttatagaaggattaaaaatatttaaagaagaaaattgtgacatgatagtaactgtaggtggaggaagttctcatgattgcggtaagggaataggaattgctgcaacacatgaaggagatctttatgattatgcaggaatagaaacacttgtcaatccattgccaccaatagtagctgtaaatactactgcaggaactgctagtgaattaactcgtcattgtgtattgactaatacaaaaaagaaaataaaatttgttatagttagctggagaaatttgcctttagtatctataaatgatccaatgcttatggttaaaaaacctgcaggattaacagcagctacaggaatggatgctttaacacatgcaatagaagcatatgtatcaaaagatgcaaatccagtaacagatgcttcagcaatacaagctattaaattaatttcacaaaatttaagacaagctgtagctttaggagaaaatcttgaagcaagagaaaatatggcttatgcatcattattagcaggaatggcatttaataatgctaatttaggatatgtacatgcaatggctcatcaattagggggactgtatgatatggcacatggtgttgctaatgcaatgctattaccacatgttgaacgttataatatgatatcaaatcctaagaagtttgcagatatagcagaatttatgggagaaaatatatctggactttctgtaatggaagcagcagagaaagccataaatgcaatgtttagactttcagaggatgttggaattccgaaaagtttaaaggagatgggagttaaacaagaagattttgagcatatggcagaactagctcttttagatggaaatgcatttagcaatccaagaaaaggaaatgcaaaagatattataaatatttttaaggctgcttattaa

SEQ ID NO: 66 - protein, 385 aa, Clostridium butyricum
MRMYDYLVPSVNFMGANSVSVVGERCKILGGKKALIVTDKFLKDMEGGAVELTVKYLKEAGLDAVYYDGVEPNPKDVNVIEGLKIFKEENCDMIVTVGGGSSHDCGKGIGIAATHEGDLYDYAGIETLVNPLPPIVAVNTTAGTASELTRHCVLTNTKKKIKFVIVSWRNLPLVSINDPMLMVKKPAGLTAATGMDALTHAIEAYVSKDANPVTDASAIQAIKLISQNLRQAVALGENLEARENMAYASLLAGMAFNNANLGYVHAMAHQLGGLYDMAHGVANAMLLPHVERYNMISNPKKFADIAEFMGENISGLSVMEAAEKAINAMFRLSEDVGIPKSLKEMGVKQEDFEHMAELALLDGNAFSNPRKGNAKDIINIFKAAY

SEQ ID NO: 67 - DNA, 1164 bp, Citrobacter freundii
atgagctatcgtatgtttgattacctggtgccaaatgtgaacttctttggccccaatgctatttccgtggtcggcgaacgctgcaaactgttgggcggtaaaaaagcgctgctggtcactgataaaggtctgcgggcgattaaagacggcgcggtagataaaaccctcacacatctgcgtgaagccggtattgacgtcgtggtttttgacggcgttgagccaaaccccaaagacaccaacgtgcgcgacggcctggaggtctttcggaaagagcattgcgacatcatcgttaccgttggcggcggtagcccgcatgactgcggtaaaggcatcggtatcgccgcgactcacgaaggggatctctacagctatgccgggattgaaaccctgaccaacccgctgccgccgatcgttgcggtgaataccaccgccggtaccgccagcgaagtcacccgccactgcgtgctgaccaataccaaaaccaaagtgaagtttgtgattgtcagctggcgcaacctgccgtcggtctccattaacgatccgctgctaatgctcggcaagccagccccactgactgcggctaccgggatggacgccctgacccacgccgtggaagcctacatttccaaagatgccaacccggtcaccgacgctgccgctatccaggcgatccgcctgatcgcccgtaacttgcgccaggccgtggcgctgggcagcaacctgaaagctcgcgagaacatggcctacgcctccctgctggcgggtatggccttcaacaacgccaacctcggctacgttcacgcgatggcgcatcagcttggcggtctttacgacatgccgcacggcgtggcgaatgccgtactgctgccgcacgtagcgcgctataacctgatcgctaacccggaaaaatttgccgacatcgcagagtttatgggcgagaacacggacggactctccaccatggatgccgccgagctggccattcatgctattgcccgcctctccgccgacatcggtattccgcagcatctgcgcgatctgggcgtcaaagaagccgatttcccgtatatggctgaaatggcactgaaggacggcaacgccttctccaacccacgcaaagggaacgagaaagaaattgccgagatcttccgtcaggcattctga

SEQ ID NO: 68 - protein, 387 aa, Citrobacter freundii
MSYRMFDYLVPNVNFFGPNAISVVGERCKLLGGKKALLVTDKGLRAIKDGAVDKTLTHLREAGIDVVVFDGVEPNPKDTNVRDGLEVFRKEHCDIIVTVGGGSPHDCGKGIGIAATHEGDLYSYAGIETLTNPLPPIVAVNTTAGTASEVTRHCVLTNTKTKVKFVIVSWRNLPSVSINDPLLMLGKPAPLTAATGMDALTHAVEAYISKDANPVTDAAAIQAIRLIARNLRQAVALGSNLKARENMAYASLLAGMAFNNANLGYVHAMAHQLGGLYDMPHGVANAVLLPHVARYNLIANPEKFADIAEFMGENTDGLSTMDAAELAIHAIARLSADIGIPQHLRDLGVKEADFPYMAEMALKDGNAFSNPRKGNEKEIAEIFRQAF

SEQ ID NO: 69 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggtggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctgatcgaagatggtccgaaagccctgaaagagccagaaaactacgatgtgcgcgccaacgtcatgtggggggcgacgcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgagaccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 70 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWGATQALNGLIGAGVPQDWATHMLGHELTAMHGLDHAQTLAIVLPALWNEKRETKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 71 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLQGTKFIAAAANYPENIDPWHILQTGGKEIKSAIPMGCVLTLPATGSESHAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWGATQALNGLIGAGVPQDWATHMLGHELTAMHGLDHAQTLAIVLPALWNEKRETKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 72 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEAYVTKPVDAKIQDRFAEGILLTLIEDGPKALKEPENYDVRANVMWGATQALNGLIGAGVPQDWATHMLGHELTAMHGLDHAQTLAIVLPALWNEKRETKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 73 - DNA, 2433 bp, Escherichia coli
atgagtgtgattgcgcaggcaggggcgaaaggtcgtcagctgcataaatttggtggcagtagtctggctgatgtgaagtgttatttgcgtgtcgcgggcattatggcggagtactctcagcctgacgatatgatggtggtttccgccgccggtagcaccactaaccagttgattaactggttgaaactaagccagaccgatcgtctctctgcgcatcaggttcaacaaacgctgcgtcgctatcagtgcgatctgattagcggtctgctacccgctgaagaagccgatagcctcattagcgcttttgtcagcgaccttgagcgcctggcggcgctgctcgacagcggtattaacgacgcagtgtatgcggaagtggtgggccacggggaagtatggtcggcacgtctgatgtctgcggtacttaatcaacaagggctgccagcggcctggcttgatgcccgcgagtttttacgcgctgaacgcgccgcacaaccgcaggttgatgaagggctttcttacccgttgctgcaacagctgctggtgcaacatccgggcaaacgtctggtggtgaccggatttatcagccgcaacaacgccggtgaaacggtgctgctggggcgtaacggttccgactattccgcgacacaaatcggtgcgctggcgggtgtttctcgcgtaaccatctggagcgacgtcgccggggtatacagtgccgacccgcgtaaagtgaaagatgcctgcctgctgccgttgctgcgtctggatgaggccagcgaactggcgcgcctggcggctcccgttcttcacgcccgtactttacagccggtttctggcagcgaaatcgacctgcaactgcgctgtagctacacgccggatcaaggttccacgcgcattgaacgcgtgctggcctccggtactggtgcgcgtattgtcaccagccacgatgatgtctgtttgattgagtttcaggtgcccgccagtcaggatttcaaactggcgcataaagagatcgaccaaatcctgaaacgcgcgcaggtacgcccgctggcggttggcgtacataacgatcgccagttgctgcaattttgctacacctcagaagtggccgacagtgcgctgaaaatcctcgacgaagcgggattacctggcgaactgcgcctgcgtcaggggctggcgctggtggcgatggtcggtgcaggcgtcacccgtaacccgctgcattgccaccgcttctggcagcaactgaaaggccagccggtcgaatttacctggcagtccgatgacggcatcagcctggtggcagtactgcgcaccggcccgaccgaaagcctgattcaggggctgcatcagtccgtcttccgcgcagaaaaacgcatcggcctggtattgttcggtaagggcaatatcggttcccgttggctggaactgttcgcccgtgagcagagcacgctttcggcacgtaccggctttgagtttgtgctggcaggtgtggtggacagccgccgcagcctgttgagctatgacgggctggacgccagccgcgcgttagccttcttcaacgatgaagcggttgagcaggatgaagagtcgttgttcctgtggatgcgcgcccatccgtatgatgatttagtggtgctggacgttaccgccagccagcagcttgctgatcagtatcttgatttcgccagccacggtttccacgttatcagcgccaacaaactggcgggagccagcgacagcaataaatatcgccagatccacgacgccttcgaaaaaaccgggcgtcactggctgtacaatgccaccgtcggtgcgggcttgccgatcaaccacaccgtgcgcgatctgatcgacagcggcgatactattttgtcgatcagcgggatcttctccggcacgctctcctggctgttcctgcaattcgacggtagcgtgccgtttaccgagctggtggatcaggcgtggcagcagggcttaaccgaacctgacccgcgtgacgatctctctggcaaagacgtgatgcgcaagctggtgattctggcgcgtgaagcaggttacaacatcgaaccggatcaggtacgtgtggaatcgctggtgcctgctcattgcgaaggcggcagcatcgaccatttctttgaaaatggcgatgaactgaacgagcagatggtgcaacggctggaagcggcccgcgaaatggggctggtgctgcgctacgtggcgcgtttcgatgccaacggtaaagcgcgtgtaggcgtggaagcggtgcgtgaagatcatccgttggcatcactgctgccgtgcgataacgtctttgccatcgaaagccgctggtatcgcgataaccctctggtgatccgcggacctggcgctgggcgcgacgtcaccgccggggcgattcagtcggatatcaaccggctggcacagttgttgtaa

SEQ ID NO: 74 - protein, 810 aa, Escherichia coli
MSVIAQAGAKGRQLHKFGGSSLADVKCYLRVAGIMAEYSQPDDMMVVSAAGSTTNQLINWLKLSQTDRLSAHQVQQTLRRYQCDLISGLLPAEEADSLISAFVSDLERLAALLDSGINDAVYAEVVGHGEVWSARLMSAVLNQQGLPAAWLDAREFLRAERAAQPQVDEGLSYPLLQQLLVQHPGKRLVVTGFISRNNAGETVLLGRNGSDYSATQIGALAGVSRVTIWSDVAGVYSADPRKVKDACLLPLLRLDEASELARLAAPVLHARTLQPVSGSEIDLQLRCSYTPDQGSTRIERVLASGTGARIVTSHDDVCLIEFQVPASQDFKLAHKEIDQILKRAQVRPLAVGVHNDRQLLQFCYTSEVADSALKILDEAGLPGELRLRQGLALVAMVGAGVTRNPLHCHRFWQQLKGQPVEFTWQSDDGISLVAVLRTGPTESLIQGLHQSVFRAEKRIGLVLFGKGNIGSRWLELFAREQSTLSARTGFEFVLAGVVDSRRSLLSYDGLDASRALAFFNDEAVEQDEESLFLWMRAHPYDDLVVLDVTASQQLADQYLDFASHGFHVISANKLAGASDSNKYRQIHDAFEKTGRHWLYNATVGAGLPINHTVRDLIDSGDTILSISGIFSGTLSWLFLQFDGSVPFTELVDQAWQQGLTEPDPRDDLSGKDVMRKLVILAREAGYNIEPDQVRVESLVPAHCEGGSIDHFFENGDELNEQMVQRLEAAREMGLVLRYVARFDANGKARVGVEAVREDHPLASLLPCDNVFAIESRWYRDNPLVIRGPGAGRDVTAGAIQSDINRLAQLL

SEQ ID NO: 75 - DNA, 2463 bp, Escherichia coli
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctga

SEQ ID NO: 76 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARIFVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 77 - DNA, 1350 bp, Escherichia coli
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 78 - protein, 449 aa, Escherichia coli
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALILDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 79 - DNA, 930 bp, Escherichia coli
atgccgattcgtgtgccggacgagctacccgccgtcaatttcttgcgtgaagaaaacgtctttgtgatgacaacttctcgtgcgtctggtcaggaaattcgtccacttaaggttctgatccttaacctgatgccgaagaagattgaaactgaaaatcagtttctgcgcctgctttcaaactcacctttgcaggtcgatattcagctgttgcgcatcgattcccgtgaatcgcgcaacacgcccgcagagcatctgaacaacttctactgtaactttgaagatattcaggatcagaactttgacggtttgattgtaactggtgcgccgctgggcctggtggagtttaatgatgtcgcttactggccgcagatcaaacaggtgctggagtggtcgaaagatcacgtcacctcgacgctgtttgtctgctgggcggtacaggccgcgctcaatatcctctacggcattcctaagcaaactcgcaccgaaaaactctctggcgtttacgagcatcatattctccatcctcatgcgcttctgacgcgtggctttgatgattcattcctggcaccgcattcgcgctatgctgactttccggcagcgttgattcgtgattacaccgatctggaaattctggcagagacggaagaaggggatgcatatctgtttgccagtaaagataagcgcattgcctttgtgacgggccatcccgaatatgatgcgcaaacgctggcgcaggaatttttccgcgatgtggaagccggactagacccggatgtaccgtataactatttcccgcacaatgatccgcaaaatacaccgcgagcgagctggcgtagtcacggtaatttactgtttaccaactggctcaactattacgtctaccagatcacgccatacgatctacggcacatgaatccaacgctggattaa

SEQ ID NO: 80 - protein, 309 aa, Escherichia coli [sequence omitted]

SEQ ID NO: 81 - DNA, 933 bp, Escherichia coli
atggttaaagtttatgccccggcttccagtgccaatatgagcgtcgggtttgatgtgctcggggcggcggtgacacctgttgatggtgcattgctcggagatgtagtcacggttgaggcggcagagacattcagtctcaacaacctcggacgctttgccgataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgttcggtggtcgcggcgctgatggcgatgaatgaacactgcggcaagccgcttaatgacactcgtttgctggctttgatgggcgagctggaaggccgtatctccggcagcattcattacgacaacgtggcaccgtgttttctcggtggtatgcagttgatgatcgaagaaaacgacatcatcagccagcaagtgccagggtttgatgagtggctgtgggtgctggcgtatccggggattaaagtctcgacggcagaagccagggctattttaccggcgcagtatcgccgccaggattgcattgcgcacgggcgacatctggcaggcttcattcacgcctgctattcccgtcagcctgagcttgccgcgaagctgatgaaagatgttatcgctgaaccctaccgtgaacggttactgccaggcttccggcaggcgcggcaggcggtcgcggaaatcggcgcggtagcgagcggtatctccggctccggcccgaccttgttcgctctgtgtgacaagccggaaaccgcccagcgcgttgccgactggttgggtaagaactacctgcaaaatcaggaaggttttgttcatatttgccggctggatacggcgggcgcacgagtactggaaaactaa

SEQ ID NO: 82 - protein, 310 aa, Escherichia coli
MVKVYAPASSANMSVGFDVLGAAVTPVDGALLGDVVTVEAAETFSLNNLGRFADKLPSEPRENIVYQCWERFCQELGKQIPVAMTLEKNMPIGSGLGSSACSVVAALMAMNEHCGKPLNDTRLLALMGELEGRISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 83 - DNA, 2652 bp, Escherichia coli
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 84 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 85 - DNA, 3465 bp, Rhizobium etli
ttgcccatatccaagatactcgttgccaatcgctctgaaatagccatccgcgtgttccgcgcggccaacgagcttggaataaaaacggtggcgatctgggcggaagaggacaagctggcgctgcaccgcttcaaggcggacgagagttatcaggtcggccgcggaccgcatcttgcccgcgacctcgggccgatcgaaagctatctgtcgatcgacgaggtgatccgcgtcgccaagctttccggtgccgacgccatccatccgggctacggcctcttgtcggaaagccccgaattcgtcgatgcctgcaacaaggccggcatcatcttcatcggcccgaaggccgatacgatgcgccagcttggcaacaaggtcgcagcgcgcaacctggcgatctcggtcggcgtaccggtcgtgccggcgaccgagccactgccggacgatatggccgaagtggcgaagatggcggcggcgatcggctatcccgtcatgctgaaggcatcctggggcggcggcggtcgcggcatgcgcgtcattcgttccgaggccgacctcgccaaggaagtgacggaagccaagcgcgaggcgatggcggccttcggcaaggacgaggtctatctcgaaaaactggtcgagcgcgcccgccacgtcgaaagccagatcctcggcgacacccacggcaatgtcgtgcatctcttcgagcgcgactgttccgttcagcgccgcaatcagaaggtcgtcgagcgcgcgcccgcaccctatctttcggaagcgcagcgccaggaactcgccgcctattcgctgaagatcgcaggggcgaccaactatatcggcgccggcaccgtcgaatatctgatggatgccgataccggcaaattttacttcatcgaagtcaatccgcgcatccaggtcgagcacacggtgaccgaagtcgtcaccggcatcgatatcgtcaaggcgcagatccacatcctggacggcgccgcgatcggcacgccgcaatccggcgtgccgaaccaggaagacatccgtctcaacggtcacgccctgcagtgccgcgtgacgacggaagatccggagcacaacttcattccggattacggccgcatcaccgcctatcgctcggcttccggcttcggcatccggcttgacggcggcacctcttattccggcgccatcatcacccgctattacgatccgctgctcgtcaaggtcacggcctgggcgccgaacccgctggaagccatttcccgcatggaccgggcgctgcgcgaattccgcatccgtggcgtcgccaccaacctgaccttcctcgaagcgatcatcggccatccgaaattccgcgacaacagctacaccacccgcttcatcgacacgacgccggagctcttccagcaggtcaagcgccaggaccgcgcgacgaagcttctgacctatctcgccgacgtcaccgtcaatggccatcccgaggccaaggacaggccgaagcccctcgagaatgccgccaggccggtggtgccctatgccaatggcaacggggtgaaggacggcaccaagcagctgctcgatacgctcggcccgaaaaaattcggcgaatggatgcgcaatgagaagcgcgtgcttctgaccgacaccacgatgcgcgacggccaccagtcgctgctcgcaacccgcatgcgtacctatgacatcgccaggatcgccggcacctattcgcatgcgctgccgaacctcttgtcgctcgaatgctggggcggcgccaccttcgacgtctcgatgcgcttcctcaccgaagatccgtgggagcggctggcgctgatccgagagggggcgccgaacctgctcctgcagatgctgctgcgcggcgccaatggcgtcggttacaccaactatcccgacaatgtcgtcaaatacttcgtccgccaggcggccaaaggcggcatcgatctcttccgcgtcttcgactgcctgaactgggtcgagaatatgcgggtgtcgatggatgcgattgccgaggagaacaagctctgcgaggcggcgatctgctacaccggcgatatcctcaattccgcccgcccgaaatacgacttgaaatattacaccaaccttgccgtcgagcttgagaaggccggcgcccatatcattgcggtcaaggatatggcgggccttctgaagccggctgctgccaaggttctgttcaaggcgctgcgtgaagcaaccggcctgccgatccatttccacacgcatgacacctcgggcattgcggcggcaacggttcttgccgccgtcgaagccggtgtcgatgccgtcgatgcggcgatggatgcgctctccggcaacacctcgcaaccctgtctcggctcgatcgtcgaggcgctctccggctccgagcgcgatcccggcctcgatccggcatggatccgccgcatctccttctattgggaagcggtgcgcaaccagtatgccgccttcgaaagcgacctcaagggaccggcatcggaagtctatctgcatgaaatgccgggcggccagttcaccaacctcaaggagcaggcccgctcgctggggctggaaacccgctggcaccaggtggcgcaggcctatgccgacgccaaccagatgttcggcgatatcgtcaaggtgacgccatcctccaaggtcgtcggcgacatggcgctgatgatggtctcccaggacctgaccgtcgccgatgtcgtcagccccgaccgcgaagtctccttcccggaatcggtcgtctcgatgctgaagggcgatctcggccagcctccgtctggatggccggaagcgctgcagaagaaagcattgaagggcgaaaagccctatacggtgcgccccggctcgctgctcaaggaagccgatctcgatgcggaacgcaaagtcatcgagaagaagcttgagcgcgaggtcagcgacttcgaattcgcttcctatctgatgtatccgaaggtcttcaccgactttgcgcttgcctccgatacctacggtccggtttcggtgctgccgacgcccgcctatttttacgggttggcggacggcgaggagctgttcgccgacatcgagaagggcaagacgctcgtcatcgtcaatcaggcggtgagcgccaccgacagccagggcatggtcactgtcttcttcgagctcaacggccagccgcgccgtatcaaggtgcccgatcgggcccacggggcgacgggagccgccgtgcgccgcaaggccgaacccggcaatgccgcccatgtcggtgcgccgatgccgggcgtcatcagccgtgtctttgtctcttcaggccaggccgtcaatgccggcgacgtgctcgtctccatcgaggccatgaagatggaaaccgcgatccatgcggaaaaggacggcaccattgccgaagtgctggtcaaggccggcgatcagatcgatgccaaggacctgctggcggtttacggcggatga

SEQ ID NO: 86 - protein, 1154 aa, Rhizobium etli
LPISKILVANRSEIAIRVFRAANELGIKTVAIWAEEDKLALHRFKADESYQVGRGPHLARDLGPIESYLSIDEVIRVAKLSGADAIHPGYGLLSESPEFVDACNKAGIIFIGPKADTMRQLGNKVAARNLAISVGVPVVPATEPLPDDMAEVAKMAAAIGYPVMLKASWGGGGRGMRVIRSEADLAKEVTEAKREAMAAFGKDEVYLEKLVERARHVESQILGDTHGNVVHLFERDCSVQRRNQKVVERAPAPYLSEAQRQELAAYSLKIAGATNYIGAGTVEYLMDADTGKFYFIEVNPRIQVEHTVTEVVTGIDIVKAQIHILDGAAIGTPQSGVPNQEDIRLNGHALQCRVTTEDPEHNFIPDYGRITAYRSASGFGIRLDGGTSYSGAIITRYYDPLLVKVTAWAPNPLEAISRMDRALREFRIRGVATNLTFLEAIIGHPKFRDNSYTTRFIDTTPELFQQVKRQDRATKLLTYLADVTVNGHPEAKDRPKPLENAARPVVPYANGNGVKDGTKQLLDTLGPKKFGEWMRNEKRVLLTDTTMRDGHQSLLATRMRTYDIARIAGTYSHALPNLLSLECWGGATFDVSMRFLTEDPWERLALIREGAPNLLLQMLLRGANGVGYTNYPDNVVKYFVRQAAKGGIDLFRVFDCLNWVENMRVSMDAIAEENKLCEAAICYTGDILNSARPKYDLKYYTNLAVELEKAGAHIIAVKDMAGLLKPAAAKVLFKALREATGLPIHFHTHDTSGIAAATVLAAVEAGVDAVDAAMDALSGNTSQPCLGSIVEALSGSERDPGLDPAWIRRISFYWEAVRNQYAAFESDLKGPASEVYLHEMPGGQFTNLKEQARSLGLETRWHQVAQAYADANQMFGDIVKVTPSSKVVGDMALMMVSQDLTVADVVSPDREVSFPESVVSMLKGDLGQPPSGWPEALQKKALKGEKPYTVRPGSLLKEADLDAERKVIEKKLEREVSDFEFASYLMYPKVFTDFALASDTYGPVSVLPTPAYFYGLADGEELFADIEKGKTLVIVNQAVSATDSQGMVTVFFELNGQPRRIKVPDRAHGATGAAVRRKAEPGNAAHVGAPMPGVISRVFVSSGQAVNAGDVLVSIEAMKMETAIHAEKDGTIAEVLVKAGDQIDAKDLLAVYGG

SEQ ID NO: 87 - DNA, 3501 bp, Ralstonia eutropha
atggactacgcccctatccgctccctgctgattgccaaccgttccgaggcgatccgcgtgatgcgcgcggccgccgagatgaacgtgcgcacggtggcaatctattcgaaggaagaccggctcgcgctccatcgcttcaaggccgatgagagctacctggtcggcgagggcaagaagccactggcggcttacctcgacatcgacgatatcctgcgcattgccaggcaggcgaaggtcgacgccattcatccgggctatggcttcctttcagagaacccggacttcgcgcaggccgtgatcgacgcgggtatccgctggatcggcccgtcgcccgaggtcatgcgcaagcttggcaacaaggtggcggcgcgcaacgcggcgatcgacgcgggcgtgccggtgatgccggcaaccgatccgctgccgcatgacctggacacgtgcaagcgcctcgccgccggcatcggctatccgctgatgctcaaggcaagctggggcggcggcggacgcggcatgcgggtcctggaacgcgagcaggaccttgagggggcgctcgccgcggcgcggcgcgaggcgctggctgcgttcggcaacgacgaggtgtatgtcgagaagctggtgcgcaacgcgcgccatgtcgaagtgcaggtgctcggcgacacgcacggcaacctcgtgcatctctatgagcgcgactgtaccgtgcagcggcgcaaccagaaggtggtggagcgggcgcccgcgccatacctcgacgatgccggccgggccgcgctgtgcgaatcggccctgcggctgatgcgcgcggtcggctacacgcatgccggtacggtcgagttcctgatggatgccgactccggccagttctacttcatcgaggtcaatccgcgcatccaggtcgagcacacggtcacggagatggtcaccgggatcgatatcgtcaaggcgcagatccgcgtgaccgaaggcggccatctcggcatgaccgagaacacgcgcaatgagaacggcgagatcgtcgtgcgcgccgcgggcgtgccggtgcaggaagcgatttcgctcaacggtcacgcgctgcaatgccggatcaccaccgaggacccggagaacgggttcctgccggactacggccgcctcactgcctaccgcagcgcggccggcttcggcgtgcgcctggacgccggcaccgcctacggcggcgcggtgatcacgccgtactacgattcgctgctggtcaaggttaccacctgggcgccgaccgcgcccgaatcgatccggcgcatggaccgcgcgctgcgcgagttccgcatccgcggcgtcgcgtccaacctgcagttcctcgagaacgtcatcaaccatccctcgttccggtccggcgacgtcaccacgcgctttatcgacctgacgccggaactgctggcgttcaccaagcgcctggaccgcgccaccaagctgctgcgctacctgggcgaggtcagcgtcaacgggcacccggagatgagcggccgcacgctgccatcgctgccgctgcccgcaccggtgctgcccgccttcgacaccggcggcgcgctgccctacggtacgcgcgaccggctgcgcgagctgggcgcggagaagttctcgcgctggatgctggagcagaagcaggtgctgctgaccgataccaccatgcgcgacgcgcaccagtcgctgttcgccacgcgcatgcgcaccgccgacatgctgccgatcgcgccgttctatgcgcgcgaactgtcgcagctgttctcgctggagtgctggggcggcgccaccttcgacgtggcgctgcgcttcctcaaggaagacccgtggcagcgccttgagcaactgcgcgagcgcgttcccaacgtgctgttccagatgctgctgcgcggctccaacgcggttggctacaccaattatgcggacaacgtggtgcgcttcttcgtgcgccaggcggccagcgccggcgtggatgtgttccgcgtgttcgattcactgaactgggtgcgcaacatgcgcgtggcgatcgatgctgtcggcgagagcggcgcgctgtgcgaaggcgcgatctgctataccggcgacctgttcgacaagtcgcgcgccaaatacgacctgaagtactacgtaggcatcgcgcgcgagctgaagcaggccggcgtgcacgtgctgggcatcaaggacatggccggcatctgccgtccgcaggccgcggcggcactggtcagggcgctcaaggaagagaccgggctgccggtgcatttccatacccacgataccagcggcatctcggccgcttcggcgctggccgcgatcgaggccggctgcgatgcggtcgacggcgcgctcgacgccatgagcgggctgacctcgcaacccaacctgtcgagcatcgccgcggccctggccggcagcgagcgcgatcccggcctcagcctggagcgcctgcacgaggcgtcgatgtactgggaaggggtgcgccgctactacgcgccgttcgaatccgaaatccgcgccggcaccgccgacgtgtaccgccacgagatgcccggcggccagtacaccaacctgcgcgagcaggcgcgctcgctcggcatcgagcatcgctggaccgaggtgtcgcgggcctatgccgaggtcaaccagatgtttggcgacatcgtcaaggtgacgccgacgtccaaggtggtcggcgacctggccttgatgatggtggccaacgacctgagcgccgccgatgtgtgcgatcccgccagggagactgccttccctgaatcggtggtgtcgctgttcaagggcgagctgggctttccgccggacggcttccccgcggaactgtcgcgcaaggtgctgcgcggcgagccgcccgtgccgtaccggcccggcgaccagatcccgccggtcgacctcgacgcggcgcgcgccgcggccgaagcggcgtgcgagcagccgctcgacgaccgccagctggcttcgtacctgatgtacccgaagcaggccggcgagtaccacgcgcatgtgcgcaactacagcgacacctcggtggtacccacgccggcatacctgtacggcctgcagccgcaggaagaagtggcgatcgacatcgctgccggcaagaccctgctggtctcgctgcaaggcacgcaccccgatgccgaagagggtgtcatcaaggtccagttcgagctgaacgggcagtcgcgcaccacgctggtcgagcagcgcagcaccacgcaagcggcggcagcgcgccatggccgtccggttgccgaacccgacaatccgctgcatgtcgccgcgcccatgccgggctcgatcgtgacggtggcggtgcagccggggcagcgcgtggccgcgggcacgacgctgctggcgctggaggcgatgaagatggaaacccatatcgcggcggagcgggactgcgagatcgccgcagtccatgttcagcagggggatcgcgtggcggcgaaggatctgctgatcgaactgaagggctga

SEQ ID NO: 88 - protein, 1167 aa, Ralstonia eutropha
MDYAPIRSLLIANRSEIAIRVMRAAAEMNVRTVAIYSKEDRLALHRFKADESYLVGEGKKPLAAYLDIDDILRIARQAKVDAIHPGYGFLSENPDFAQAVIDAGIRWIGPSPEVMRKLGNKVAARNAAIDAGVPVMPATDPLPHDLDTCKRLAAGIGYPLMLKASWGGGGRGMRVLEREQDLEGALAAARREALAAFGNDEVYVEKLVRNARHVEVQVLGDTHGNLVHLYERDCTVQRRNQKVVERAPAPYLDDAGRAALCESALRLMRAVGYTHAGTVEFLMDADSGQFYFIEVNPRIQVEHTVTEMVTGIDIVKAQIRVTEGGHLGMTENTRNENGEIVVRAAGVPVQEAISLNGHALQCRITTEDPENGFLPDYGRLTAYRSAAGFGVRLDAGTAYGGAVITPYYDSLLVKVTTWAPTAPESIRRMDRALREFRIRGVASNLQFLENVINHPSFRSGDVTTRFIDLTPELLAFTKRLDRATKLLRYLGEVSVNGHPEMSGRTLPSLPLPAPVLPAFDTGGALPYGTRDRLRELGAEKFSRWMLEQKQVLLTDTTMRDAHQSLFATRMRTADMLPIAPFYARELSQLFSLECWGGATFDVALRFLKEDPWQRLEQLRERVPNVLFQMLLRGSNAVGYTNYADNVVRFFVRQAASAGVDVFRVFDSLNWVRNMRVAIDAVGESGALCEGAICYTGDLFDKSRAKYDLKYYVGIARELKQAGVHVLGIKDMAGICRPQAAAALVRALKEETGLPVHFHTHDTSGISAASALAAIEAGCDAVDGALDAMSGLTSQPNLSSIAAALAGSERDPGLSLERLHEASMYWEGVRRYYAPFESEIRAGTADVYRHEMPGGQYTNLREQARSLGIEHRWTEVSRAYAEVNQMFGDIVKVTPTSKVVGDLALMMVANDLSAADVCDPARETAFPESVVSLFKGELGFPPDGFPAELSRKVLRGEPPVPYRPGDQIPPVDLDAARAAAEAACEQPLDDRQLASYLMYPKQAGEYHAHVRNYSDTSVVPTPAYLYGLQPQEEVAIDIAAGKTLLVSLQGTHPDAEEGVIKVQFELNGQSRTTLVEQRSTTQAAAARHGRPVAEPDNPLHVAAPMPGSIVTVAVQPGQRVAAGTTLLALEAMKMETHIAAERDCEIAAVHVQQGDRVAAKDLLIELKG

SEQ ID NO: 89 - DNA, 888 bp, Escherichia coli
ggatccatgtctagaatgagccaagccctgaaaaacctgctgacgctgctgaatctggaaaaaatcgaagaaggcctgttccgtggtcaatctgaagacctgggcctgcgtcaggtgtttggcggtcaggtggttggtcaagcgctgtatgcggccaaagaaaccgttccggaagaacgtctggtccatagctttcactcttatttcctgcgcccgggcgatagcaaaaaaccgattatctacgatgtggaaaccctgcgcgacggcaacagtttttccgcccgtcgcgttgcagctattcagaatggtaaaccgatcttttacatgacggcatcattccaggcaccggaagctggctttgaacatcaaaaaaccatgccgagcgccccggcaccggatggtctgccgagtgaaacgcagattgcacaatccctggctcatctgctgccgccggtcctgaaagataaatttatctgtgaccgtccgctggaagtccgtccggtggaatttcacaacccgctgaaaggccatgtcgcagaaccgcaccgtcaagtgtggattcgcgctaatggcagcgtgccggatgacctgcgtgttcatcaatatctgctgggttacgcgtctgatctgaactttctgccggttgccctgcaaccgcacggcattggtttcctggaaccgggtattcaaatcgccacgatcgaccattcaatgtggtttcaccgcccgttcaacctgaatgaatggctgctgtattccgttgaatcaaccagcgcgagcagcgcccgtggctttgtccgtggtgaattttacacgcaagatggtgtcctggtggcgtctaccgttcaagaaggcgttatgcgtaatcacaactaagagctcaagctt

SEQ ID NO: 90 - protein, 286 aa, Escherichia coli
MSQALKNLLTLLNLEKIEEGLFRGQSEDLGLRQVFGGQVVGQALYAAKETVPEERLVHSFHSYFLRPGDSKKPIIYDVETLRDGNSFSARRVAAIQNGKPIFYMTASFQAPEAGFEHQKTMPSAPAPDGLPSETQIAQSLAHLLPPVLKDKFICDRPLEVRPVEFHNPLKGHVAEPHRQVWIRANGSVPDDLRVHQYLLGYASDLNFLPVALQPHGIGFLEPGIQIATIDHSMWFHRPFNLNEWLLYSVESTSASSARGFVRGEFYTQDGVLVASTVQEGVMRNHN

SEQ ID NO: 91 - DNA, 1602 bp, Clostridium propionicum
ggatccatgtctagaatgcgcaaagtcccgattattacggcagatgaagcggctaaactgattaaagacggcgatacggtcaccaccagcggtttcgttggcaacgcaattccggaagctctggatcgtgcggttgaaaaacgctttctggaaaccggcgaaccgaaaaacatcacgtatgtctactgcggcagtcagggtaatcgtgatggccgcggtgccgaacatttcgcacacgaaggcctgctgaaacgttatattgctggtcattgggccaccgtcccggcactgggtaaaatggcaatggaaaacaaaatggaagcgtataatgtgtcacagggcgcgctgtgtcacctgtttcgtgatattgcctcgcacaaaccgggtgtctttaccaaagtgggcattggtacgtttatcgacccgcgcaacggcggtggcaaagtgaatgatattaccaaagaagacatcgtcgaactggtggaaattaaaggccaggaatacctgttttatccggcgttcccgattcatgttgccctgatccgcggcacctatgccgatgaatctggtaacattacgtttgaaaaagaagtggcaccgctggaaggcaccagcgtgtgccaggcagtcaaaaattctggtggcatcgtggttgtccaagttgaacgtgtggttaaagcgggcaccctggacccgcgccacgttaaagtcccgggtatttatgtggactacgtcgtggttgctgatccggaagaccatcagcaaagtctggattgtgaatatgatccggcactgtccggtgaacaccgtcgcccggaagttgtgggtgaaccgctgccgctgagtgctaaaaaagttattggccgtcgcggtgcgatcgaactggaaaaagatgtggccgttaacctgggcgtgggtgcaccggaatacgttgcgtccgtcgccgatgaagaaggcattgttgactttatgaccctgacggcagatagcggtgctattggcggcgtgccggcgggcggcgttcgttttggcgcgtcttataatgcggatgccctgatcgaccagggttaccaattcgattattacgacggtggcggtctggatctgtgctatctgggcctggcggaatgtgacgaaaagggtaacattaatgtgtcacgttttggtccgcgtattgcgggttgtggtggtttcattaacatcacccagaatacgccgaaagtctttttctgtggcacctttacggcaggcggtctgaaagtgaaaattgaagatggcaaagtgattatcgttcaggaaggtaaacagaaaaaattcctgaaagcggttgaacaaatcaccttcaacggtgatgtcgcactggctaataaacagcaagtgacctatatcacggaacgttgcgtttttctgctgaaagaagatggcctgcacctgtcggaaattgcgccgggtattgatctgcaaacccaaattctggatgtgatggacttcgccccgattatcgatcgcgacgcaaatggccagatcaaactgatggatgcggcactgtttgcggaaggtctgatgggtctgaaagaaatgaaatcgtaagagctcaagctt

SEQ ID NO: 92 - protein, 524 aa, Clostridium propionicum
MRKVPIITADEAAKLIKDGDTVTTSGFVGNAIPEALDRAVEKRFLETGEPKNITYVYCGSQGNRDGRGAEHFAHEGLLKRYIAGHWATVPALGKMAMENKMEAYNVSQGALCHLFRDIASHKPGVFTKVGIGTFIDPRNGGGKVNDITKEDIVELVEIKGQEYLFYPAFPIHVALIRGTYADESGNITFEKEVAPLEGTSVCQAVKNSGGIVVVQVERVVKAGTLDPRHVKVPGIYVDYVVVADPEDHQQSLDCEYDPALSGEHRRPEVVGEPLPLSAKKVIGRRGAIELEKDVAVNLGVGAPEYVASVADEEGIVDFMTLTADSGAIGGVPAGGVRFGASYNADALIDQGYQFDYYDGGGLDLCYLGLAECDEKGNINVSRFGPRIAGCGGFINITQNTPKVFFCGTFTAGGLKVKIEDGKVIIVQEGKQKKFLKAVEQITFNGDVALANKQQVTYITERCVFLLKEDGLHLSEIAPGIDLQTQILDVMDFAPIIDRDANGQIKLMDAALFAEGLMGLKEMKS

SEQ ID NO: 93 - DNA, 1581 bp, Megasphaera elsdenii
ggatccatgtctagaatgcgtaaagttgaaattattaccgcagaacaggcagcacagctggttaaagataatgataccattaccagcattggctttgttagcagcgcacatccggaagcactgaccaaagcactggaaaaacgttttctggataccaatacaccgcagaatctgacctatatttatgcaggtagccagggtaaacgtgatggtcgtgcagcagaacatctggcacatacaggtctgctgaaacgtgcaattattggtcattggcagaccgttccggcaattggtaaactggcagtggaaaataaaattgaagcctataattttagccagggcaccctggttcattggtttcgtgcactggcaggtcataaactgggtgtttttaccgatattggcctggaaacctttctggacccgcgtcagctgggtggtaaactgaatgatgttaccaaagaggatctggttaaactgattgaagtggatggtcatgaacagctgttttatccgacctttccggttaatgttgcatttctgcgtggcacctatgcagatgaaagcggtaatattacaatggatgaagaaattggtccgtttgaaagcaccagcgttgcacaggcagttcataattgtggtggtaaagttgtggttcaggttaaagatgttgttgcacatggtagcctggacccgcgtatggttaaaattccgggtatttatgtggattatgttgttgttgcagcaccggaagatcatcagcagacctatgattgtgaatatgatccgagcctgagcggtgaacatcgtgcaccggaaggtgcagcagatgcagcactgccgatgagcgcaaaaaaaattattggtcgtcgtggtgcactggaactgaccgaaaatgcagttgttaatctgggtgttggtgcaccggaatatgttgcaagcgttgcgggtgaagaaggtattgcagataccattacactgaccgttgatggtggtgcaattggtggtgttccgcagggtggtgcacgttttggtagcagccgtaatgcagatgccattattgatcatacctatcagtttgatttttatgatggtggtggtctggatattgcatatctgggtctggcacagtgtgatggtagtggtaatattaatgtgagcaaatttggcaccaatgttgcaggttgtggtggttttccgaatattagccagcagaccccgaatgtttatttttgtggcacctttaccgcaggcggtctgaaaattgcagttgaagatggcaaagtgaaaattctgcaagaaggcaaagccaaaaaatttattaaagccgtggatcagattacctttaatggtagctatgcagcccgtaatggtaaacatgttctgtatattaccgaacgctgcgtttttgaactgacaaaagaaggtctgaaactgatcgaagttgcaccgggtattgatattgaaaaagatattctggcccacatggattttaaaccgattattgataatccgaaactgatggatgcccgtctgtttcaggatggtccgatgggtctgaaacgttaagagctcaagctt

SEQ ID NO: 94 - protein, 516 aa, Megasphaera elsdenii
MRKVEIITAEQAAQLVKDNDTITSIGFVSSAHPEALTKALEKRFLDTNTPQNLTYIYAGSQGKRDGRAAEHLAHTGLLKRIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGAADAALPMSAKKIIGRRGALELTENAVVNLGVGAPEYVASVAGEEGIADTITLTVDGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKR

SEQ ID NO: 95 - DNA, 1077 bp, Saccharomyces cerevisiae
ggatccatgtctagaatgtctgcgagcaaaatggcgatgagcaacctggaaaaaattctggaactggtgccgctgtctccgacctcctttgtgacgaaatacctgccggcggcaccggttggctctaaaggcaccttcggcggtacgctggtcagccagtccctgctggccagtctgcataccgtgccgctgaactttttcccgacgtctctgcacagttatttcattaaaggcggtgacccgcgtaccaaaatcacgtaccatgttcagaacctgcgtaatggccgcaactttattcataaacaggtctccgcttatcaacacgataaactgatttttacctcaatgatcctgttcgcggttcagcgtagcaaagaacatgatagcctgcaacactgggaaaccatcccgggcctgcaaggtaaacaaccggacccgcaccgctacgaagaagcgacgtcgctgtttcagaaagaagtgctggacccgcaaaaactgtcacgttatgcgtcactgtcggatcgcttccaggacgccaccagcatgtctaaatacgtcgatgcatttcagtatggcgtgatggaataccaatttccgaaagacatgttctatagcgcccgtcatacggatgaactggactacttcgtgaaagttcgcccgccgattaccacggtcgaacatgcaggtgatgaaagctctctgcacaaacatcacccgtatcgtattccgaaaagcatcaccccggaaaatgatgctcgctataactacgtggcatttgcttatctgagtgactcctacctgctgctgaccattccgtattttcataatctgccgctgtactgccactcattcagcgtgagcctggatcatacgatctattttcatcagctgccgcacgttaacaattggatttacctgaaaatctccaacccgcgttcacattgggataaacacctggttcagggcaaatattttgacacccaatctggtcgcatcatggcgagtgtctcccaggaaggctatgtggtgtatggtagtgaacgtgatattcgtgccaaattctaagagctcaagctt

SEQ ID NO: 96 - protein, 349 aa, Saccharomyces cerevisiae
MSASKMAMSNLEKILELVPLSPTSFVTKYLPAAPVGSKGTFGGTLVSQSLLASLHTVPLNFFPTSLHSYFIKGGDPRTKITYHVQNLRNGRNFIHKQVSAYQHDKLIFTSMILFAVQRSKEHDSLQHWETIPGLQGKQPDPHRYEEATSLFQKEVLDPQKLSRYASLSDRFQDATSMSKYVDAFQYGVMEYQFPKDMFYSARHTDELDYFVKVRPPITTVEHAGDESSLHKHHPYRIPKSITPENDARYNYVAFAYLSDSYLLLTIPYFHNLPLYCHSFSVSLDHTIYFHQLPHVNNWIYLKISNPRSHWDKHLVQGKYFDTQSGRIMASVSQEGYVVYGSERDIRAKF

SEQ ID NO: 97 - DNA, 990 bp, Mus musculus
ggatccatgtctagaatgtctgccccggaaggcctgggtgatgcacacggtgatgctgatcgcggtgacctgagcggtgacctgcgttcggttctggttacgagcgtcctgaacctggaaccgctggatgaagacctgtatcgtggccgccattactgggttccgacctctcagcgtctgtttggcggtcagattatgggtcaagccctggtcgcggccgcaaaaagcgtgtctgaagatgtccatgtgcactcactgcattgctattttgttcgtgccggcgatccgaaagttccggtcctgtaccacgtcgaacgtatccgcacgggtgcaagtttctccgtgcgtgctgttaaagcggtccagcatggtaaagccatttttatctgtcaggcaagtttccagcaaatgcaaccgtccccgctgcaacaccaattttcaatgccgtcggtgccgccgccggaagacctgctggatcatgaagcgctgattgatcagtatctgcgtgacccgaacctgcacaaaaaataccgtgtcggtctgaatcgcgtggctgcgcaagaagttccgattgaaatcaaagtggttaacccgccgaccctgacgcagctgcaagctctggaaccgaaacagatgttctgggtgcgtgcgcgcggctatattggcgaaggtgatatcaaaatgcattgctgtgttgccgcatatatctcagactacgcttttctgggcaccgccctgctgccgcaccagagcaaatataaagtgaatttcatggccagcctggatcattctatgtggtttcacgccccgttccgcgcagaccattggatgctgtacgaatgcgaatccccgtgggctggcggttttcgcggtctggttcatggtcgcctgtggcgtcgcgatggtgtgctggcagttacctgtgcccaagaaggcgtcatccgtctgaaaccgcaagtgtctgaatctaaactgtgagagctcaagctt

SEQ ID NO: 98 - protein, 320 aa, Mus musculus
MSAPEGLGDAHGDADRGDLSGDLRSVLVTSVLNLEPLDEDLYRGRHYWVPTSQRLFGGQIMGQALVAAAKSVSEDVHVHSLHCYFVRAGDPKVPVLYHVERIRTGASFSVRAVKAVQHGKAIFICQASFQQMQPSPLQHQFSMPSVPPPEDLLDHEALIDQYLRDPNLHKKYRVGLNRVAAQEVPIEIKVVNPPTLTQLQALEPKQMFWVRARGYIGEGDIKMHCCVAAYISDYAFLGTALLPHQSKYKVNFMASLDHSMWFHAPFRADHWMLYECESPWAGGFRGLVHGRLWRRDGVLAVTCAQEGVIRLKPQVSESKL

SEQ ID NO: 99 - DNA, 1698 bp, Rattus norvegicus
ggatccatgtctagaatggaaccgaccgtggccccgggtgaagtgctgatgtcgcaagctatccaaccggctcatgcagattcgcgtggtgaactgtccgctggccagctgctgaaatggatggataccaccgcatgcctggcggccgaaaaacatgcaggtatctcttgtgttaccgctagtatggatgacattctgtttgaagacacggcacgcattggccagatcgtcaccattcgcgcgaaagtgacgcgtgccttttccacctcaatggaaatttcgatcaaagtccgtgtgcaggataaattcacgggcattcaaaaactgctgtgcgtggcgttttctaccttcgtggttaaaccgctgggtaaagaaaaagtgcatctgaaaccggttctgctgcaaacggaacaggaacaagtcgaacatcgcctggcctccgaacgtcgcaaagtgcgtctgcaacacgaaaacaccttttccaatatcatgaaagaatcaaactggctgcgcgatccggtgtgtaatgaagaagaaggcaccgcgaccacgatggccacctccgttcagtcaattgaactggtcctgccgccgcacgcaaaccatcacggtaatacctttggcggtcaaatcatggcttggatggaaacggttgcaaccatttcggctagccgtctgtgccatggccacccgttcctgaaatccgtggatatgtttaaatttcgtggtccgtcaacggttggtgaccgtctggtgtttaacgcgattgttaacaataccttccagaacagcgttgaagtcggtgtgcgcgttgaagcgtttgattgtcgtgaatgggccgaaggccagggtcgccatatcaacagtgcgttcctgatttataatgccgttgatgaccaggaagaactgatcacctttccgcgtatccaaccgattagcaaagatgacttccgtcgctatcagggtgccattgcacgtcgccgtattcgcctgggtcgtaaatacgtcatttcgcacaaaaaagaagtgccgctgggcacgcagtgggatatcagcaaaaaaggttctattagtaacaccaatgtggaagcactgaaaaacctggcttccaaatcaggctgggaaatcaccacgaccctggaaaaaatcaaaatctataccctggaagaacaggatgcgatttctgtcaaagtggaaaaacaagtgggtagtccggcacgcgttgcttatcatctgctgagcgattttaccaaacgtccgctgtgggacccgcactacatctcgtgcgaagttattgatcaagtcagcgaagatgaccaaatctattacattacgtgttctgtcgtgaacggtgataaaccgaaagacttcgttgtcctggttagccagcgcaaaccgctgaaagatgacaatacctatattgtggcactgatgtccgtggttctgccgtcggttccgccgagcccgcagtacatccgttcacaagtgatttgcgctggctttctgatccagccggttgatagctctagttgtaccgtcgcgtatctgaaccaaatgtcggacagcattctgccgtacttcgccggtaatatcggcggttggtctaaaagtattgaagaagcagctgcgagttgtatcaaattcatcgaaaacgctacgcacgacggtctgaaatcggttctgtaagagctcaagctt

SEQ ID NO: 100 - protein, 556 aa, Rattus norvegicus
MEPTVAPGEVLMSQAIQPAHADSRGELSAGQLLKWMDTTACLAAEKHAGISCVTASMDDILFEDTARIGQIVTIRAKVTRAFSTSMEISIKVRVQDKFTGIQKLLCVAFSTFVVKPLGKEKVHLKPVLLQTEQEQVEHRLASERRKVRLQHENTFSNIMKESNWLRDPVCNEEEGTATTMATSVQSIELVLPPHANHHGNTFGGQIMAWMETVATISASRLCHGHPFLKSVDMFKFRGPSTVGDRLVFNAIVNNTFQNSVEVGVRVEAFDCREWAEGQGRHINSAFLIYNAVDDQEELITFPRIQPISKDDFRRYQGAIARRRIRLGRKYVISHKKEVPLGTQWDISKKGSISNTNVEALKNLASKSGWEITTTLEKIKIYTLEEQDAISVKVEKQVGSPARVAYHLLSDFTKRPLWDPHYISCEVIDQVSEDDQIYYITCSVVNGDKPKDFVVLVSQRKPLKDDNTYIVALMSVVLPSVPPSPQYIRSQVICAGFLIQPVDSSSCTVAYLNQMSDSILPYFAGNIGGWSKSIEEAAASCIKFIENATHDGLKSVL

SEQ ID NO: 101 - DNA, 756 bp, Bacillus subtilis
ggatccatgtctagaatgtcacaactgttcaaatccttcgatgcttcagaaaaaacccaactgatttgcttcccgttcgcaggtggctattccgcctccttccgtccgctgcatgcgtttctgcaaggcgaatgtgaaatgctggcggccgaaccgccgggtcatggcaccaaccagacgagcgccattgaagacctggaagaactgaccgacctgtataaacaggaactgaatctgcgtccggatcgcccgtttgtgctgttcggccattctatgggcggtatgattacgtttcgtctggcacagaaactggaacgcgaaggtatcttcccgcaagcagttattatcagtgctattcagccgccgcatatccaacgtaaaaaagtctcccacctgccggatgaccagtttctggatcatattatccaactgggcggtatgccggcggaactggtcgaaaacaaagaagtgatgtcatttttcctgccgagctttcgttctgattatcgcgcactggaacagtttgaactgtacgacctggctcagatccaatcgccggtgcacgtttttaatggcctggatgacaaaaaatgtattcgcgatgcggaaggttggaaaaaatgggccaaagatatcacctttcatcagttcgacggcggtcacatgttcctgctgtcccaaacggaagaagtggcagaacgcattttcgctatcctgaaccagcatccgattatccagccgtaagagctcaagctt

SEQ ID NO: 102 - protein, 242 aa, Bacillus subtilis
MSQLFKSFDASEKTQLICFPFAGGYSASFRPLHAFLQGECEMLAAEPPGHGTNQTSAIEDLEELTDLYKQELNLRPDRPFVLFGHSMGGMITFRLAQKLEREGIFPQAVIISAIQPPHIQRKKVSHLPDDQFLDHIIQLGGMPAELVENKEVMSFFLPSFRSDYRALEQFELYDLAQIQSPVHVFNGLDDKKCIRDAEGWKKWAKDITFHQFDGGHMFLLSQTEEVAERIFAILNQHPIIQP

SEQ ID NO: 103 - DNA, 1602 bp, Clostridium propionicum
ggatccatgtctagaatgcgcaaagtcccgattattacggcagatgaagcggctaaactgattaaagacggcgatacggtcaccaccagcggtttcgttggcaacgcaattccggaagctctggatcgtgcggttgaaaaacgctttctggaaaccggcgaaccgaaaaacatcacgtatgtctactgcggcagtcagggtaatcgtgatggccgcggtgccgaacatttcgcacacgaaggcctgctgaaacgttatattgctggtcattgggccaccgtcccggcactgggtaaaatggcaatggaaaacaaaatggaagcgtataatgtgtcacagggcgcgctgtgtcacctgtttcgtgatattgcctcgcacaaaccgggtgtctttaccaaagtgggcattggtacgtttatcgacccgcgcaacggcggtggcaaagtgaatgatattaccaaagaagacatcgtcgaactggtggaaattaaaggccaggaatacctgttttatccggcgttcccgattcatgttgccctgatccgcggcacctatgccgatgaatctggtaacattacgtttgaaaaagaagtggcaccgctggaaggcaccagcgtgtgccaggcagtcaaaaattctggtggcatcgtggttgtccaagttgaacgtgtggttaaagcgggcaccctggacccgcgccacgttaaagtcccgggtatttatgtggactacgtcgtggttgctgatccggaagaccatcagcaaagtctggattgtgaatatgatccggcactgtccggtgaacaccgtcgcccggaagttgtgggtgaaccgctgccgctgagtgctaaaaaagttattggccgtcgcggtgcgatcgaactggaaaaagatgtggccgttaacctgggcgtgggtgcaccggaatacgttgcgtccgtcgccgatgaagaaggcattgttgactttatgaccctgacggcagaaagcggtgctattggcggcgtgccggcgggcggcgttcgttttggcgcgtcttataatgcggatgccctgatcgaccagggttaccaattcgattattacgacggtggcggtctggatctgtgctatctgggcctggcggaatgtgacgaaaagggtaacattaatgtgtcacgttttggtccgcgtattgcgggttgtggtggtttcattaacatcacccagaatacgccgaaagtctttttctgtggcacctttacggcaggcggtctgaaagtgaaaattgaagatggcaaagtgattatcgttcaggaaggtaaacagaaaaaattcctgaaagcggttgaacaaatcaccttcaacggtgatgtcgcactggctaataaacagcaagtgacctatatcacggaacgttgcgtttttctgctgaaagaagatggcctgcacctgtcggaaattgcgccgggtattgatctgcaaacccaaattctggatgtgatggacttcgccccgattatcgatcgcgacgcaaatggccagatcaaactgatggatgcggcactgtttgcggaaggtctgatgggtctgaaagaaatgaaatcgtaagagctcaagctt

SEQ ID NO: 104 - protein, 524 aa, Clostridium propionicum; identical to SEQ ID NO: 46

SEQ ID NO: 105 - DNA, 1581 bp, Megasphaera elsdenii
ggatccatgtctagaatgcgtaaagttgaaattattaccgcagaacaggcagcacagctggttaaagataatgataccattaccagcattggctttgttagcagcgcacatccggaagcactgaccaaagcactggaaaaacgttttctggataccaatacaccgcagaatctgacctatatttatgcaggtagccagggtaaacgtgatggtcgtgcagcagaacatctggcacatacaggtctgctgaaacgtgcaattattggtcattggcagaccgttccggcaattggtaaactggcagtggaaaataaaattgaagcctataattttagccagggcaccctggttcattggtttcgtgcactggcaggtcataaactgggtgtttttaccgatattggcctggaaacctttctggacccgcgtcagctgggtggtaaactgaatgatgttaccaaagaggatctggttaaactgattgaagtggatggtcatgaacagctgttttatccgacctttccggttaatgttgcatttctgcgtggcacctatgcagatgaaagcggtaatattacaatggatgaagaaattggtccgtttgaaagcaccagcgttgcacaggcagttcataattgtggtggtaaagttgtggttcaggttaaagatgttgttgcacatggtagcctggacccgcgtatggttaaaattccgggtatttatgtggattatgttgttgttgcagcaccggaagatcatcagcagacctatgattgtgaatatgatccgagcctgagcggtgaacatcgtgcaccggaaggtgcagcagatgcagcactgccgatgagcgcaaaaaaaattattggtcgtcgtggtgcactggaactgaccgaaaatgcagttgttaatctgggtgttggtgcaccggaatatgttgcaagcgttgcgggtgaagaaggtattgcagataccattacactgaccgttgaaggtggtgcaattggtggtgttccgcagggtggtgcacgttttggtagcagccgtaatgcagatgccattattgatcatacctatcagtttgatttttatgatggtggtggtctggatattgcatatctgggtctggcacagtgtgatggtagtggtaatattaatgtgagcaaatttggcaccaatgttgcaggttgtggtggttttccgaatattagccagcagaccccgaatgtttatttttgtggcacctttaccgcaggcggtctgaaaattgcagttgaagatggcaaagtgaaaattctgcaagaaggcaaagccaaaaaatttattaaagccgtggatcagattacctttaatggtagctatgcagcccgtaatggtaaacatgttctgtatattaccgaacgctgcgtttttgaactgacaaaagaaggtctgaaactgatcgaagttgcaccgggtattgatattgaaaaagatattctggcccacatggattttaaaccgattattgataatccgaaactgatggatgcccgtctgtttcaggatggtccgatgggtctgaaacgttaagagctcaagctt

SEQ ID NO: 106 - protein, 517 aa, Megasphaera elsdenii
MRKVEIITAEQAAQLVKDNDTITSIGFVSSAHPEALTKALEKRFLDTNTPQNLTYIYAGSQGKRDGRAAEHLAHTGLLKRAIIGHWQTVPAIGKLAVENKIEAYNFSQGTLVHWFRALAGHKLGVFTDIGLETFLDPRQLGGKLNDVTKEDLVKLIEVDGHEQLFYPTFPVNVAFLRGTYADESGNITMDEEIGPFESTSVAQAVHNCGGKVVVQVKDVVAHGSLDPRMVKIPGIYVDYVVVAAPEDHQQTYDCEYDPSLSGEHRAPEGAADAALPMSAKKIIGRRGALELTENAVVNLGVGAPEYVASVAGEEGIADTITLTVEGGAIGGVPQGGARFGSSRNADAIIDHTYQFDFYDGGGLDIAYLGLAQCDGSGNINVSKFGTNVAGCGGFPNISQQTPNVYFCGTFTAGGLKIAVEDGKVKILQEGKAKKFIKAVDQITFNGSYAARNGKHVLYITERCVFELTKEGLKLIEVAPGIDIEKDILAHMDFKPIIDNPKLMDARLFQDGPMGLKR

SEQ ID NO: 107 - DNA, 438 bp, Haemophilus influenzae
ggatccatgtctagaatgctggataattgctttagctttccggtgcgtgtgtattatgaagataccgatgccggtggtgttgtttatcatgcacgttatctgcatttttttgaacgtgcacgtaccgaatatctgcgtaccctgaattttacccagcagaccctgctggaagaacagcagctggcatttgttgttaaaaccctggcaattgattattgcgttgcagcaaaactggatgatctgctgatggttgaaaccgaagttagcgaagttaaaggtgcaaccattctgtttgaacagcgtctgatgcgtaataccctgatgctgagcaaagcaaccgttaaagttgcatgtgttgatctgggtaaaatgaaaccggttgcctttccgaaagaagttaaagcagcatttcatcatctgaaataagagctcaagctt

SEQ ID NO: 108 - protein, 136 aa, Haemophilus influenzae
MLDNCFSFPVRVYYEDTDAGGVVYHARYLHFFERARTEYLRTLNFTQQTLLEEQQLAFVVKTLAIDYCVAAKLDDLLMVETEVSEVKGATILFEQRLMRNTLMLSKATVKVACVDLGKMKPVAFPKEVKAAFHHLK

SEQ ID NO: 109 - protein, 523 aa, Haemophilus influenzae
MGKVKVLTARQAADLIKDGDTVTLSGFVANGIAEALNAAAEERFLETGHPKDLTLFWVAGTGNKDGSHADHYAHEGMVKKVIGGHFNFVPKICEMLSENKIEGYNVPQGAIAQMLRDNAARKVGTISHVGIGTFADPRNGGGRLSEKTKEDIVKIIELEGQEQLFYPRIPLDVAFIRGTYADELGNITLEKEMAPLDATSQAMAVHNNGGLVVVQVERVVKAGHLDPKLVKIPGIYVDAVVECPADDPKQSQSINCTYDPAYAGNTQVPVSSLEPKKLDAKKIIGRRAAMELKKNVVVNLGVGVPEWVSSVAAEEGVADEMTLTVDCGPVGGVPGGGLRFGGSVNAQAYMDEGYQFDFYDGGGLDLCFLGLAEVDNNGDVNVSRLGTRITGSGGFTNISSNSKKAVFCGTFTNGVKIQTGDGKLTILEEGKKHKFVNKVTEITFSGVVAGKAGKDVLYVTERAVFALKADGIHLIEVAPGIDVQTQVLDEMDFAPIVDRDADGNVKLMDARIFKDEVMGMTID

SEQ ID NO: 110 - protein, 527 aa, Listeria monocytogenes
MSKVIKASEAAKLLKDGDTVAFSGFGLACVNEEMAIAVEKRFLEEGAPRNLTVMHASALGDRREKGMSHWGHEGLIKRWIGGIAIASPKMAKLIEEDKCEAYNLPQGVITQLYREIAAKRPGVITKIGMGTFVDPRIEGAKMSASSKDNLVELLTIHNEEWLFYPSFPIQVALIRGTVADEFGNLTLEKEGLHMEVLPIAQAVRNSGGIVIAQVESVAKKGSLNPKDVRVPGILVDHIIISEPENHFQTENTQYNPAFSGHIQVPLGDIEPLPLDDRKVIARRSAAELEPQTILNLGVGIPVNVSTVAAEEGVSDQLILTTDAGSVGGVPAGLADFGHAYNSEAIVDHHSQFDFYDGGGLDLSVLGLAQTDESGNVNVSKFGSRVAGCGGFINISQSAKKLIFAGTFTAGGLKTRVADGKLEILQEGKAKKFIKQVQQITFSGEYASTTNQTILYVTERAVFRLENGKMVLTEIAPGIDLEKDILGQMEFEPIIASDLKVMDGGMFSEEWGGLKAIIEKQTREGVSI

SEQ ID NO: 111 - protein, 472 aa, Pseudomonas mendocina
PRDLTLVYAAGQGDGKGRGLNHLAHEGLVRRVIGGHWGLVPGLQKLAVDNRIEAYNLPQGVISQLFRDIAAGKPGQLSRVGLGTYVDPRHGGGKLNARTTADLVRLMPIDGEDYLFYPTFPIDVAVVRATSSDPDGNLSFEREALTIESLAIAMAARNCGGLVIAQVERIVERGSLNAREVKIPGILVDCVVQAEPANHQQTFATAYNPAFAAETRVPVDSLAPMPLDVRKLIARRAALELKGGAVVNLGIGMPDGVAAVAAEEGVIERLTLTAEPGVIGGVPASGLDFGAASNHSALLDQPYQFDFYDGGGLDIAFLGLAQADAAGNLNVSKFGSRLAGAGGFINISQNAKQVVFVGTFSAGAQDIRIEGGQLRIVQDGELRKFVAEVEHRTFAGRLAAERGQPVLYVTERCVLRLSREGLELIEVAPGVDIQRDILSRMDFAPIVREPKLMDARLFRPEPIGLAQCLENL

SEQ ID NO: 112 - protein, 515 aa, Unknown
  Erysipelotrichaceae bacterium
MSKVISIEQAVSMIPDGAAIGIGGFIGSGHPQEFSVGIEESFLKSGHPKDLTIMFSAGIGDGTDRLGLNKLGHEGLLKRIIGGHWGLIPKLQKLVFENKVEGYNLPLGTISLMFRDIAGHRPGTITKVGLKTFVDPRIEGAKMNERSKEDLVELMHIDGEEWLRYKSFPLNVALIRATYCDEDGNATMEKEAATLDSLSIAQAAKNSGGIVLLQVEKVVQNGTLDPRKVKIPGIYVDGIVVARPENHWQTYANPYDPALSGEVKVPVNSIAPMKLNERKVICRRAAMELDPAAIINLGIGMPDGIANVANEEGLPGLKLTVEAGGIGGVPNAGTAFGTCTNPDAIIDQPYQFDFYDGGGLDQAFLGLAECDCSGNINVSRFGPKIAGCGGFINITQTSPVVVYCGTFTAGGLKVEVRDGKLHILQEGRIKKFKKEVEQITFSAEFATETGQKVLYVTERAVFELLDGKLTLTEIAPGVDLEQDVLGQMEFKPAVAEHLKTMDERLFRDELMGLKA

SEQ ID NO: 113 - DNA, 1263 bp, Escherichia coli
atgccacattcactgttcagcaccgataccgatctcaccgccgaaaatctgctgcgtttgcccgctgaatttggctgcccggtgtgggtctacgatgcgcaaattattcgtcggcagattgcagcgctgaaacagtttgatgtggtgcgctttgcacagaaagcctgttccaatattcatattttgcgcttaatgcgtgagcagggcgtgaaagtggattccgtctcgttaggcgaaatagagcgtgcgttggcggcgggttacaatccgcaaacgcaccccgatgatattgtttttacggcagatgttatcgatcaggcgacgcttgaacgcgtcagtgaattgcaaattccggtgaatgcgggttctgttgatatgctcgaccaactgggccaggtttcgccagggcatcgggtatggctgcgcgttaatccggggtttggtcacggacatagccaaaaaaccaataccggtggcgaaaacagcaagcacggtatctggtacaccgatctgcccgccgcactggacgtgatacaacgtcatcatctgcagctggtcggcattcacatgcacattggttctggcgttgattatgcccatctggaacaggtgtgtggtgctatggtgcgtcaggtcatcgaattcggtcaggatttacaggctatttctgcgggcggtgggctttctgttccttatcaacagggtgaagaggcggttgataccgaacattattatggtctgtggaatgccgcgcgtgagcaaatcgcccgccatttgggccaccctgtgaaactggaaattgaaccgggtcgcttcctggtagcgcagtctggcgtattaattactcaggtgcggagcgtcaaacaaatggggagccgccactttgtgctggttgatgccgggttcaacgatctgatgcgcccggcaatgtacggtagttaccaccatatcagtgccctggcagctgatggtcgttctctggaacacgcgccaacggtggaaaccgtcgtcgccggaccgttatgtgaatcgggcgatgtctttacccagcaggaagggggaaatgttgaaacccgcgccttgccggaagtgaaggcaggtgattatctggtactgcatgatacaggggcatatggcgcatcaatgtcatccaactacaatagccgtccgctgttaccagaagttctgtttgataatggtcaggcgcggttgattcgccgtcgccagaccatcgaagaattactggcgctggaattgctttaa

SEQ ID NO: 114 - protein, 420 aa, Escherichia coli
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 115 - DNA, 879 bp, Escherichia coli
atgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaa

SEQ ID NO: 116 - protein, 292 aa, Escherichia coli
MFTGSIVAIVTPMDEKGNVCRASLKKLIDYHVASGTSAIVSVGTTGESATLNHDEHADVVMMTLDLADGRIPVIAGTGANATAEAISLTQRFNDSGIVGCLTVTPYYNRPSQEGLYQHFKAIAEHTDLPQILYNVPSRTGCDLLPETVGRLAKVKNIIGIKEATGNLTRVNQIKELVSDDFVLLSGDDASALDFMQLGGHGVISVTANVAARDMAQMCKLAAEGHFAEARVINQRLMPLHNKLFVEPNPIPVKWACKELGLVATDTLRLPMTPITDSGRETVRAALKHAGLL

SEQ ID NO: 117 - DNA, 410 bp, Artificial sequence
  modification to pet30a vector
gcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccccctagaaataattttgtttaactttaagaaggagatatacatatgcaccatcatcatcatcattcttctggtaccggtggtggctccggtattgagggtcgcgccatggcgatatcgaattcggatccgagctccctgcagctcgag

SEQ ID NO: 118 - DNA, 33 bp, Artificial sequence
  5′ prefix sequence immediately upstream of the start codon
ggtaccggtggtggctccggtattgagggtcgc

SEQ ID NO: 119 - DNA, 21 bp, Artificial Sequence
  3′ suffix sequence immediately downstream of the stop codon.
tactagtagcggccgctgcag

SEQ ID NO: 120 - DNA, 1242 bp, Sus scrofa
atggctccgccgagtgtgtttgctgaagttccgcaggcccaaccggtgctggtgtttaagctgattgctgattttcgtgaagacccggacccgcgtaaagttaatctgggcgtcggtgcatatcgcaccgatgactgccagccgtgggtgctgccggtggttcgtaaggttgaacaacgcattgcgaacgatagctctctgaatcatgaatacctgccgatcctgggcctggccgaatttcgtacctgtgcaagccgcctggctctgggtgatgactctccggcgctgcaagaaaaacgtgtcggcggtgtgcagagcctgggcggcaccggcgctctgcgtattggtgcggaatttctggcccgctggtataacggcaccaacaataaagacacgccggtttacgtcagttccccgacctgggaaaaccacaatggcgtgtttaccaccgcgggcttcaaagatattcgtagctatcgctactgggatacggaaaagcgcggcctggatctgcaaggttttctgagtgatctggaaaatgcgccggaattttccattttcgtcctgcatgcatgcgcacacaacccgaccggcaccgatccgaccccggaacagtggaaacaaatcgccagtgttatgaagcgtcgctttctgttcccgtttttcgactcagcgtatcagggctttgcctcgggtaatctggaaaaagatgcatgggctattcgttacttcgtttctgaaggttttgaactgttctgtgcacagagctttagcaaaaacttcggcctgtataatgaacgcgttggtaacctgaccgtcgtggctaaagaaccggatagtattctgcgtgtcctgtcccagatggaaaagatcgtgcgcgttacctggtcaaatccgccggcacaaggcgcccgtattgtggcacgtacgctgtcggacccggaactgtttcatgaatggaccggtaacgttaaaacgatggccgatcgtatcctgagcatgcgctctgaactgcgtgcacgcctggaagctctgaagaccccgggtacgtggaaccatattaccgaccagatcggcatgtttagcttcacgggtctgaatccgaaacaagtggaatatctgattaacgaaaagcacatctacctgctgccgagcggtcgtattaatatgtgcggtctgaccacgaaaaacctggattatgtcgcaacctctattcacgaagctgtgacgaagatccagtaa

SEQ ID NO: 121 - DNA, 1491 bp, Rattus norvegicus
atggcgtcccgtgtcaacgatcaatcacaggcgtctcgcaatggtctgaaaggcaaggtgctgaccctggatacgatgaacccgtgtgtccgtcgcgtggaatatgcagtgcgtggtccgatcgttcagcgcgccctggaactggaacaggaactgcgtcaaggtgtcaaaaagccgtttaccgaagtgattcgcgcaaacatcggtgatgcacaggcgatgggccaacgtccgattacgtttttccgtcaggtcctggcgctgtgcgtgtacccgaacctgctgagcagcccggatttcccggaagacgcaaaacgtcgcgctgaacgtattctgcaagcgtgcggtggtcatagcctgggcgcttattctattagttccggtattcagccgatccgcgaagatgtggcacaatacattgaacgtcgcgatggtggtatcccggctgacccgaacaatatttttctgagtaccggcgcgtccgacgccatcgttacgatgctgaaactgctggtcagcggtgaaggtcgtgcgcgcaccggtgttctgattccgatcccgcagtatccgctgtactctgcggccctggcggaactggatgcagtgcaggttgattattacctggacgaagaacgtgcatgggcgctggatattgccgaactgcgtcgcgcactgtgccaggctcgtgaccgttgctgtccgcgcgttctgtgtgtcattaacccgggcaatccgaccggtcaggtccaaacgcgtgaatgcattgaagcagtgatccgctttgctttcaaggaaggcctgtttctgatggcagatgaagtttatcaggacaacgtctacgctgaaggctcacaatttcattcgttcaaaaaggtgctgatggaaatgggtccgccgtatagtacccagcaagaactggcgtcctttcactcagtgagcaaaggttatatgggcgaatgcggtttccgtggcggttacgttgaagtggttaatatggatgccgaagtccagaaacaaatgggcaagctgatgagcgtgcgcctgtgtccgccggttccgggtcaggccctgatggacatggtcgtgtctccgccgaccccgagcgaaccgtcttttaaacagttccaagcagaacgtcaggaagtgctggcggaactggcagctaaagccaagctgacggaacaggtgtttaacgaagcgccgggcattcgttgcaatccggttcagggtgccatgtatagcttcccgcaggtgcaactgccgctgaaagcggttcagcgcgcccaagaactgggtctggcgccggatatgtttttctgcctgtgtctgctggaagaaaccggcatctgtgttgtcccgggctccggttttggccagcaggaaggcacctatcatttccgtatgacgattctgccgccgatggaaaaactgcgcctgctgctggaaaagctgtcacattttcacgcgaaattcacgcacgaatactcgtaa

SEQ ID NO: 122 - DNA, 1020 bp, Pseudomonas fluorescens
atgggtaatgaatcaatcaactgggacaaactgggcttcgactacatcaagacggacaaacgctttctgcaagtgtggaaaaacggtgaatggcaggcgggcaccctgacggatgacaacgtgctgcatatcagtgaaggttccaccgcactgcactatggccagcaatgctttgaaggtctgaaagcgtatcgttgtaaggatggctcaattaacctgttccgtccggaccagaatgcggcccgtatgcaacgtagctgcgcgcgtctgctgatgccgcatgtttctaccgaagattttatcgacgcgtgtaaacaggtggttaaggccaacgaacgcttcattccgccgtatggttccggcggtgcgctgtacctgcgtccgtttgtgatcggcaccggtgataatattggcgttcgcacggccccggaatttatcttcagcgtgtttgcaattccggttggtgcttatttcaaaggcggtctggttccgcacaactttcagattagcaccttcgaccgtgcagctccgcaaggcacgggtgcagcaaaggtcggcggtaattatgcagctagtctgatgccgggcgcagaagctaaaaagtccggttttgcggatgccatctacctggacccgatgacccattcaaaaattgaagaagtgggctcggcaaactttttcggtatcacccacgataataaattcattacgccgaaaagcccgtctgttctgccgggcatcacccgtctgagcctgattgaactggccaaaacgcgcctgggcctggaagtcgtggaaggtgaagtctttattgataaactggaccagttcaaggaagcaggtgcttgcggcaccgcggcggtgattagcccgatcggcggtattcaatacaacggcaaactgcatgtttttcactcggaaaccgaagtcggtccgatcacgcagaaactgtacaaggaactgaccggcgtccaaacgggtgatgtggaagcgccggcgggttggattgttaaagtctaa

SEQ ID NO: 123 - DNA, 1218 bp, Escherichia coli
atgagcccgattgaaaagagcagcaaactggaaaacgtctgttatgacatccgtggtccggtcctgaaagaagcaaaacgcctggaagaagaaggcaacaaagtgctgaagctgaacattggcaatccggcaccgtttggtttcgatgctccggacgaaattctggtggacgttatccgtaatctgccgaccgcgcagggctattgcgatagtaaaggtctgtactccgcacgcaaggctattatgcagcattatcaagcccgtggtatgcgcgacgtcacggtggaagatatttacatcggcaacggtgtctcagaactgatcgtgcaggcgatgcaggcgctgctgaacagcggtgacgaaatgctggtgccggcaccggattatccgctgtggaccgcggcggtgagcctgagcagcggtaaagccgttcattacctgtgtgatgaaagttccgactggtttccggatctggatgacattcgtgcaaagatcaccccgcgtacgcgcggcattgttattatcaacccgaacaatccgaccggtgctgtttattccaaagaactgctgatggaaattgtcgaaatcgcacgccagcacaatctgatcatcttcgctgacgaaatttatgataaaatcctgtacgatgacgcggaacatcacagcattgcaccgctggctccggatctgctgaccatcacgtttaacggcctgtctaaaacctatcgtgtcgcaggcttccgccaaggttggatggtgctgaatggtccgaaaaagcatgctaagggctacattgaaggtctggaaatgctggcgtctatgcgtctgtgcgcaaacgtgccggcacagcacgcaatccaaaccgcactgggcggttatcagtcaatttcggaatttatcacgccgggcggtcgcctgtacgaacaacgtaaccgcgcgtgggaactgattaatgatattccgggcgttagctgtgtcaaaccgcgtggtgcgctgtatatgtttccgaaaattgatgccaagcgcttcaacatccacgatgaccagaaaatggttctggattttctgctgcaagaaaaggtgctgctggttcaaggcaccgcctttaattggccgtggccggatcatttccgtattgttacgctgccgcgcgtcgatgacatcgaactgagcctgtctaaatttgcacgtttcctgagtggttaccaccaactgtaa

SEQ ID NO: 124 - DNA, 1699 bp, Lactococcus lactis
gaattcgcggccgcttctagaaggagatatacatatgtataccgtgggtgactacctgctggaccgtctgcatgaactgggcattgaagaaatctttggtgttccgggtgactacaacctgcaatttctggatcaaattatctcacgtgaagacatgaaatggattggtaacgcaaatgaactgaacgcatcgtatatggctgatggctacgcgcgcaccaaaaaagcggcggcgtttctgaccacgttcggcgttggtgaactgagcgcgattaacggcctggccggttcttatgcagaaaatctgccggtggttgaaatcgttggctcaccgacgtcgaaagtccagaatgatggtaaatttgtgcatcacaccctggcggatggcgactttaaacatttcatgaaaatgcacgaaccggtgacggctgcgcgtaccctgctgacggcggaaaacgccacctatgaaattgatcgtgtgctgagtcaactgctgaaagaacgcaaaccggtttacatcaatctgccggttgacgtcgccgcagctaaagctgaaaaaccggcgctgtccctggaaaaagaaagctctaccacgaacaccacggaacaggttattctgagcaaaatcgaagaatctctgaaaaatgcccaaaaaccggtcgtgattgcaggccatgaagtgatcagttttggtctggaaaaaaccgtcacgcagttcgtgtccgaaaccaaactgccgattaccacgctgaactttggtaaaagcgccgtggatgaaagcctgccgtctttcctgggcatttataacggtaaactgagtgaaatctccctgaaaaacttcgtcgaatctgctgatttcatcctgatgctgggcgtgaaactgaccgacagttccacgggtgcctttacccatcacctggatgaaaacaaaatgattagcctgaatatcgacgaaggcatcatcttcaacaaagttgtcgaagatttcgacttccgtgcggtggtttcatcgctgtctgaactgaaaggcattgaatatgaaggccagtacatcgataaacaatacgaagaatttatcccgagcagcgcaccgctgagtcaggaccgtctgtggcaagcagttgaatcactgacgcagtcgaacgaaaccattgtcgctgaacaaggcaccagctttttcggtgcgtccaccatctttctgaaaagtaattcccgtttcattggtcagccgctgtggggcagcatcggttatacctttccggcggccctgggctcacaaattgccgataaagaatcgcgccatctgctgttcatcggcgacggcagcctgcaactgaccgttcaagaactgggtctgtcgattcgtgaaaaactgaacccgatctgctttattatcaacaatgatggctacacggtggaacgcgaaattcacggtccgacccagagttataacgacatcccgatgtggaattactccaaactgccggaaacgtttggcgcaaccgaagatcgtgtcgtgagcaaaattgtgcgcaccgaaaacgaatttgtgtctgttatgaaagaagcacaggctgatgttaatcgcatgtattggatcgaactggtcctggaaaaagaagatgctccgaaactgctgaaaaaaatgggtaaactgttcgctgaacaaaataaataatactagtagcggccgctgcag

SEQ ID NO: 125 - DNA, 1450 bp, Salmonella enterica
gaattcgcggccgcttctagaaggagatatacatatgaacacctcggaactggaaaccctgattcgcaccatcctgtcggaacaactgaccaccccggctcaaaccccggtccaaccgcagggcaaaggtatctttcagagcgtttctgaagcaattgatgcggcccatcaggcgtttctgcgttatcagcaatgcccgctgaaaacgcgtagcgctattatctctgcgatgcgtcaggaactgaccccgctgctggctccgctggcggaagaaagtgcgaacgaaaccggcatgggtaacaaagaagataaattcctgaagaacaaggcagctctggataatacgccgggtgtcgaagacctgaccacgaccgcactgaccggtgatggtggtatggtgctgtttgaatatagcccgttcggtgtgattggcagtgttgcaccgtccaccaacccgacggaaaccattatcaacaatagtatctccatgctggcggcgggcaacagcatttacttttcgccgcatccgggcgcgaaaaaggtttcactgaaactgatttcgctgatcgaagaaattgcctttcgttgctgtggtatccgcaacctggtggttacggtggccgaaccgacgtttgaagcaacccagcaaatgatggctcacccgcgtatcgcagtcctggcaattaccggcggtccgggcattgtggcgatgggtatgaaaagcggcaaaaaggttatcggtgcaggtgcaggtaatccgccgtgcattgttgatgaaaccgccgacctggtcaaagcggcggaagatattatcaacggtgcctcttttgactataatctgccgtgtatcgcagaaaagagcctgattgtcgtggaatctgtcgcggaacgtctggtgcagcaaatgcagacgttcggcgcgctgctgctgtccccggcggataccgacaaactgcgtgcagtttgcctgccggagggtcaggccaacaaaaagctggtcggcaaatcaccgtcggcaatgctggaagcggcgggtatcgctgtgccggcaaaggctccgcgtctgctgattgccctggtgaatgcagatgacccgtgggttacctctgaacaactgatgccgatgctgccggttgtcaaagtgagcgattttgactctgcgctggccctggcactgaaggttgaagaaggcctgcatcacaccgcgattatgcacagtcagaacgtttcccgtctgaatctggcagctcgcacgctgcaaacctcaatcttcgtcaaaaacggtccgtcgtacgcaggtattggcgtgggcggtgaaggctttacgaccttcaccatcgcaacgccgaccggtgaaggcacgaccagtgctcgtacgtttgcgcgctcccgtcgctgtgtgctgaccaatggtttcagcattcgctaatactagtagcggccgctgcag

SEQ ID NO: 126 - DNA, 1825 bp, Cupriavidus necator
gaattcgcggccgcttctagaaggagatatacatatggcaaccggcaagggcgcagcagcatccacgcaggaaggcaaatcccaaccgttcaaagttaccccgggtccgttcgatccggctacctggctggaatggagtcgccagtggcagggtacggagggtaacggtcatgcggcggcgagcggtattccgggcctggatgcgctggccggtgtgaaaattgcaccggctcaactgggcgatattcagcaacgttacatgaaggacttcagcgcgctgtggcaagcgatggccgaaggcaaggcagaagctaccggtccgctgcacgatcgtcgcttcgcaggtgacgcatggcgtacgaacctgccgtatcgttttgctgcggccttctacctgctgaatgcgcgcgccctgaccgaactggcagatgctgtcgaagcggacgccaaaacgcgtcagcgcattcgttttgcgatcagtcaatgggtggatgccatgtccccggcaaacttcctggctaccaatccggaagcacagcgtctgctgattgaaagtggcggtgaatccctgcgcgcgggtgttcgtaacatgatggaagacctgacccgcggcaagatcagccagacggacgaatctgcctttgaagtgggtcgtaacgtggccgttaccgaaggcgcagtggtttttgaaaacgaatacttccaactgctgcaatacaaaccgctgacggataaggttcatgcgcgtccgctgctgatggtcccgccgtgcatcaacaagtactacattctggatctgcaaccggaaagctctctggttcgccatgtcgtggaacaaggtcacaccgtgtttctggttagttggcgtaatccggatgcatcaatggctggctcgacgtgggatgactatattgaacatgcagctattcgcgcgatcgaagtggcccgtgatatttctggtcaggacaaaatcaatgtcctgggcttctgcgtgggcggcaccatcgttagcacggcactggctgtcctggcggcccgcggtgaacatccggcagcttctgtgaccctgctgaccacgctgctggattttgccgacacgggtattctggatgtcttcgtggacgaaggccacgttcaactgcgtgaagcaaccctgggcggtggcgcaggtgccccgtgtgccctgctgcgcggcctggaactggcaaacacctttagcttcctgcgtccgaacgatctggtgtggaattatgttgtggataactacctgaaaggcaataccccggttccgtttgatctgctgttctggaacggtgacgcaacgaatctgccgggtccgtggtattgctggtatctgcgccatacctatctgcaaaatgaactgaaagtcccgggcaagctgacggtgtgtggcgttccggtcgatctggcgtcaattgacgttccgacctatatctacggttcgcgtgaagatcacatcgtcccgtggaccgcggcctacgcgtcaacggccctgctggcaaacaagctgcgctttgtgctgggtgcttcgggccatattgcgggcgttatcaacccgccggcgaaaaataagcgtagccactggaccaatgatgccctgccggaatctccgcagcaatggctggcaggtgctattgaacatcacggctcctggtggccggattggaccgcttggctggcgggtcaggcaggtgccaaacgcgcagctccggcgaactatggtaatgcacgctaccgtgctatcgaaccggcaccgggccgttatgtgaaagcaaaggcttaatactagtagcggccgctgcag

SEQ ID NO: 127 - DNA, 835 bp, Metallosphaera sedula
gaattcgcggccgcttctagaaggagatatacatatggaatttgaaaccattgaaacgaaaaaggaaggcaacctgttctggatcaccctgaatcgcccggacaagctgaacgcactgaatgcaaaactgctggaagaactggatcgtgccgtgagtcaggccgaatccgacccggaaatccgcgttattatcattaccggtaaaggcaaggctttttgcgcaggtgctgatattacccagttcaaccaactgacgccggcggaagcctggaaattttccaaaaagggtcgtgaaatcatggacaaaattgaagcgctgagcaagccgaccatcgcgatgattaacggctatgccctgggcggtggcctggaactggcactggcttgtgatattcgtattgcggcggaagaagcgcaactgggtctgccggaaatcaatctgggtatttatccgggctacggtggcacccaacgtctgacgcgcgtgatcggtaaaggccgtgccctggaaatgatgatgaccggtgatcgcattccgggcaaagacgcagaaaagtacggcctggttaaccgtgtggttccgctggctaatctggaacaagaaacgcgcaaactggcagaaaagattgctaaaaagagcccgatctctctggcgctgattaaagaagtcgtgaatcgtggtctggattcaccgctgctgtcgggcctggccctggaaagcgtcggttggggcgttgtcttctctaccgaagacaaaaaggaaggtgttagtgcctttctggaaaaacgcgaaccgacgttcaaaggcaagtaatactagtagcggccgctgcag